(19) United States
(12) Patent Application Publication
(10) Pub. No.: US 2013/0281398 A1
(43) Pub. Date: Oct. 24, 2013

(54) TREATMENT OF DISEASES BY EPIGENETIC REGULATION

(71) Applicants: Kevin G. McLure, Calgary (CA); Peter R. Young, San Francisco, CA (US)

(72) Inventors: Kevin G. McLure, Calgary (CA); Peter R. Young, San Francisco, CA (US)

(73) Assignee: RVX Therapeutics Inc., Calgary (CA)

(21) Appl. No.: 13/832,625

(22) Filed: Mar. 15, 2013

Related U.S. Application Data
(60) Provisional application No. 61/635,739, filed on Apr. 19, 2012.

(51) Int. Cl. C07D 239/91; A61K 31/519; A61K 45/06; C07D 401/12; C07D 403/04; C07D 405/12; C07D 403/12; C07D 413/12; A61K 31/472; C07D 217/24; A61K 31/517; C07D 471/04

(52) U.S. Cl. CPC C07D 239/91; A61K 31/517; A61K 31/519; A61K 45/06; C07D 401/12; C07D 471/04; C07D 405/12; C07D 403/12; C07D 413/12; A61K 31/472; C07D 217/24; C07D 403/04
USPC 514/43; 514/266.31; 514/266.21; 514/252.17; 514/264.1; 514/266.24; 514/266.23; 514/266.2; 514/64; 514/171; 514/110; 514/309; 514/291

(57) ABSTRACT

The present disclosure provides non-naturally occurring polyphenol compounds that inhibit the bromodomain and extra terminal domain (BET) proteins. The disclosed compositions and methods can be used for treatment and prevention of diseases or disorders that are susceptible to administration of a BET inhibitor.

### TECHNICAL FIELD

The present disclosure relates to a method for inhibiting BET (bromodomain and extra terminal domain) proteins.

### BACKGROUND

Cancer is a group of diseases caused by dysregulated cell proliferation. Therapeutic approaches aim to decrease the numbers of cancer cells by inhibiting cell replication or by inducing cancer cell differentiation or death, but there is still significant unmet medical need for more efficacious therapeutic agents. Cancer cells accumulate genetic and epigenetic changes that alter cell growth and metabolism in order to promote cell proliferation and increased resistance to programmed cell death, or apoptosis. Some of these changes include inactivation of tumor suppressor genes, activation of oncogenes, as well as modifications of the regulation of chromatin structure. Watson, Cancer Discovery 1:477-480 (2011); Morin et al., Nature 476:298-303 (2011).

Many modifications of histones in chromatin have been characterized, including acetylation at multiple lysines in histones H3 and H4. Peserico and Simone, J. Biomed. Biotechnol. 2011:371832 (2011). Histone acetylation is controlled by acetylases (HATs) as well as deacetylases (HDACs), and small molecule HDAC inhibitors have been developed with cancer as an indication. Hoshino and Matsubara, Surg. Today 40:809-815 (2010). Histone acetylation controls gene expression by recruiting protein complexes that bind directly to acetylated lysine via bromodomains. Sanchez and Zhou, Curr. Opin. Drug Discov. Devel. 12(5):659-665 (2009). One such family, the bromodomain and extra terminal domain (BET) proteins, comprises Brd2, Brd3, Brd4, and BrdT each of which contains two bromodomains in tandem that can independently bind to acetylated lysines. Wu and Chiang, J. Biol. Chem. 282(18):13141-13145 (2007). BET proteins exert some of their effects on transcription by recruiting the positive transcription elongation factor b (p-TEFb), which stimulates transcription elongation by phosphorylating the C-terminal domain of RNA polymerase II and results in increased expression of growth promoting genes, such as, for example, c-Myc and the well established cancer target Aurora B. Filippakopoulos et al., Nature 468:1067-1073 (2010).

Molecules that bind to BET proteins and prevent them from binding to chromatin, inhibit transcription and prevent cell replication, which is useful in cancer therapy and other settings. For example, it has been shown that BET proteins can be displaced from the chromatin by small molecule inhibitors, such as, for example, JQ1, I-BET, and I-BET151, which specifically compete with the acetyl-lysine binding pocket of the BET protein bromodomains thereby preventing transcription elongation of their target genes. Filippakopoulos et al. (2010); Nicodeme et al., Nature 468:1119-1123 (2010); Dawson et al., Nature 478:529-533 (2011).

Inhibition of BET bromodomain-promoter interactions results in a subsequent reduction of myc transcription and protein levels. This results in G₁ arrest and extensive apoptosis in a variety of leukemia and lymphoma cell lines. Mertz et al., PNAS 108(40):16669-16674 (2011). The Myc family of proto-oncogenes (c-myc, I-myc, n-myc) is activated in 25-35% of all human cancers. Vita and Henrickson, Seminars in Cancer Biol. 16:318-330 (2006). Mouse models of cancer driven by overexpression of c-myc demonstrate that transiently inhibiting c-myc expression can cause tumor regression, cell death, and in some cancers such as leukemia, complete disease remission. Soucek et al., Nature 455:679-683 (2008). The absence of a clear ligand-binding domain of c-myc has made the development of an inhibitor a formidable challenge, thus alternative strategies to targeting c-myc transcription must be developed. Delmore et al., Cell 146:904-917 (2011). A mouse model of aggressive human medulloblastoma, in which c-myc is overexpressed, suggests that BET inhibitors may be useful for treating myc-amplified medulloblastoma. Kawauchi et al., Cancer Cell 21:168-180 (2012); Pei et al., Cancer Cell 21:155-167 (2012). Similarly, inhibition of n-myc through RNA interference significantly reduced tumor growth in neuroblastoma mouse models. Jiang et al., Biochem. Biophs. Res. Commun. 410:364-370 (2011). A similar role for 1-myc was suggested in small cell lung carcinoma cell lines using antisense oligonucleotides to inhibit 1-myc amplification. Dosaka-Akita et al., Cancer Res. 55:1559-1564 (1995). Therefore BET inhibitors have potential to be efficacious in treating multiple types of cancer.

In fact, small molecules that target the bromodomains of BET family members have demonstrated potential therapeutic use in treating cancer. See, for example, Dawson et al. (2011), showing that a small molecule inhibitor of the BET family has a profound efficacy against human and murine mixed lineage leukemia (MLL)-fusion cell lines by early cell cycle arrest and apoptosis. Its mechanism of efficacy is the selective abrogation of Brd3/4 recruitment to chromatin. BET inhibitor JQ1 has demonstrated potent antitumor activity in murine xenograoft models of NUT (nuclear protein in testis) midline carcinoma (NMC), a rare but lethal form of cancer. NMC tumor cell growth is driven by a translocation of the Brd4 gene to the nutlin 1 gene. Filippakopoulos et al., (2010). JQ1 was also shown to be a potent antiproliferator in multiple myeloma, associated with cell cycle arrest and cellular senescence. Delmore et al. (2011).

BET inhibitors are also expected to be potential therapeutics for other types of cancer. For example, in acute myeloid leukemia (AML), Brd4 is required to sustain myc expression and continued disease progression. Zuber et al., Nature 478:524-8 (2011). Moreover, inactivation of Brd4 results in a rapid and drastic down-regulation of the transcription of the proto-oncogenes c-myc and n-myc in cell lines they are amplified. Dawson et al. (2011); Delmore et al. (2011); Zuber et al. (2011); Mertz et al. (2011). Consequently, treatment of tumors that are characterized by activation or overexpression of c-myc with a BET inhibitor resulted in tumor regression through inactivation of c-myc transcription. BET inhibitors are also expected to have application in multiple myeloma, as the multiple myeloma SET domain (MMSET) which is implicated in this disease also binds to BET proteins. Dawson et al. (2011).

In addition to cancer, BET inhibitors are also expected to have have anti-inflammatory and immunomodulatory properties. Lamotte et al., Bioorganic & Med. Chem. Letters (Feb. 24, 2012); Prinjha et al., Trends Pharmacol. Sci. 33(3):146-153 (2012). BET inhibitors I-BET and I-BET151 decrease IL-6 expression in vivo. I-BET was shown to confer protection against lipopolysaccharide-induced endotoxic shock and bacteria-induced sepsis and I-BET151 was shown to suppress bacterial-induced inflammation and sepsis in a murine model. Nicodeme et al. (2010); Lamotte et al. (2012). In addition, BET inhibitors may modulate responses to viral and bacterial infections, including HIV, herpes, and papilloma viruses.

### DESCRIPTION OF THE INVENTION

The present invention provides methods of inhibiting BET proteins. The methods may be used to treat or prevent diseases or disorders that are sensitive to a compound that binds to bromodomains of BET family proteins, including cancers such as NUT midline carcinoma, as well as cancers that exhibit c-myc overexpression, including, but not limited to, Burkitt's lymphoma, acute myelogenous leukemia, multiple myeloma, aggressive human medulloblastoma; cancers overexpressing n-myc, cancers that rely on the recruitment of p-TEFb to regulate activated oncogenes such as, for example, NOTCH1. In some embodiments, BET inhibitors may induce apoptosis in cancer cells by decreasing expression of the anti-apoptosis gene Bcl2. In certain embodiments, the methods of the invention are used to treat or prevent cancers, including hematological, epithelial including lung, breast and colon carcinomas, midline carcinomas, mesenchymal, hepatic, renal and neurological tumours.

The methods of invention include administering to a mammal, such as a human, for the purpose of inhibiting BET proteins in the mammal, a therapeutically effective amount of at least one compound of Formula I:

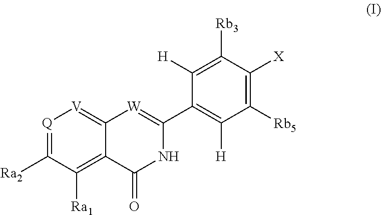

or stereoisomer, tautomer, pharmaceutically acceptable salt, or hydrate thereof, wherein:

Q is selected from N and CRa₃;

V is selected from N and CRa₄;

W is selected from N and CH;

X is selected from OH, SH, NH₂, S(O)H, S(O)₂H, S(O)₂NH₂, S(O)NH₂, NHAc, and NHSO₂Me;

Ra₁, Ra₃, and Ra₄ are independently selected from hydrogen, C₁-C₆ alkyl, C₁-C₆ alkoxy, C₃-C₆ cycloalkyl, and halogen;

Ra₂ is selected from hydrogen, C₁-C₆ alkyl, C₁-C₆ alkoxy, C₃-C₆ cycloalkyl, amino, amide, and halogen;

Rb₃ and Rb₅ are independently selected from hydrogen, halogen, C₁-C₆ alkyl, C₃-C₆ cycloalkyl, and C₁-C₆ alkoxy;

provided that at least one of Ra₁, Ra₂, Ra₃, and Ra₄ is not hydrogen.

In certain embodiments, the method of inhibiting BET proteins comprises administering a therapeutically effective amount of at least one compound of Formula II:

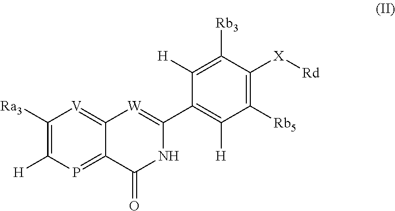

or stereoisomer, tautomer, pharmaceutically acceptable salt, or hydrate thereof, wherein:

P is selected from N and CRa₁;

V is selected from N and CH;

W is selected from N and CH;

X is selected from O, S, CH₂, and NH;

Ra₁ and Ra₃ are independently selected from hydrogen, C₁-C₆ alkyl, C₁-C₆ alkoxy, C₃-C₆ cycloalkyl, and halogen;

Rb₃ and Rb₅ are independently selected from hydrogen, C₁-C₆ alkyl, C₃-C₆ cycloalkyl, C₁-C₆ alkoxy, halogen, and amino;

Rd is selected from C₁-C₆ alkyl, C₁-C₆ alkoxy, and C₃-C₆ cycloalkyl, wherein Rd may be connected to Rb₃ or Rb₅ to form a heterocycle;

provided that:

at least one of Ra₁ and Ra₃ is not hydrogen; and

if —XRd is —OCH₂CH₂OH, then Rb₃ is not pyrrolidine.

The invention also provides methods of using a pharmaceutical composition comprising one or more compounds selected from Formula I or Formula II, or a stereoisomer, tautomer, pharmaceutically acceptable salt, or hydrate of compounds of Formula I or II, together with at least one pharmaceutically acceptable carrier, adjuvant, and/or excipient to inhibit BETproteins.

In certain embodiments, the methods of the invention are useful for the prevention or treatment of diseases that benefit from increased cell death or differentiation, or decreased cell proliferation. This may occur by, for example, decreased expression of a Myc family member or an oncogene required for tumor growth, or increase of a tumor suppressor gene, the latter antagonized by BET proteins. The method of the invention can be used to increase cancer cell death or decrease cancer cell proliferation, including, for example, by decreasing expression of Myc family member. Decreasing expression of the Myc family member may refer to, but is not limited to, transcriptionally modulating the expression of its gene or genes that have been either amplified in the genome or translocated to another chromosomal location, or transcriptionally altered in order to increase its expression (i.e. overexpression) thereby affecting the level of the c-myc protein produced. A decrease in the Myc family member mRNA levels may decrease proliferation of cancer cells and/or increase cancer cell death, including but not limited to apoptosis.

In other embodiments, the methods of the invention are useful for the prevention or treatment of diseases such as cancer in combination with other drugs. In some embodiments, a compound of Formula I or Formula II may be administered in combination with a standard of care drug(s) for any given tumor type, including, but not limited to, bortezomib, thalidomide, dexamethasone, 5-azacitidine, decitabine, vorinostat, or cyclophosphamide in multiple myeloma. In another embodiment, a compound of Formula I may be administered in combination with a PI3K or mTOR inhibitor such as rapamycin. Similarly, a compound of Formula I could be administered in combination with gamma secretase inhibitors which inhibit NOTCH1 (given the relationship between c-myc and NOTCH1) or AMPK inducers such as metformin or phenformin for leukemia. Another example of a potentially useful combination is combining a BET inhibitor which decreases myc expression, with an ornithine decarboxylase inhibitor such as difluoromethylornithine that inhibits a myc target.

In certain embodiments, the methods of the invention provide treatment of auto-immune and inflammatory diseases or conditions by administering compound of Formula I. In other embodiments, the compounds of Formula I or Formula II may be employed to treat other diseases caused by bacterial or viral infection, such as, for example, infection by HIV, HPV, or herpes virus. Certain embodiments of the invention provide, for use of a compound of Formula I or Formula II in the manufacture of a medicament it inhibit BET proteins in a subject.

### DEFINITIONS

As used in the present specification, the following words, phrases and symbols are generally intended to have the meanings as set forth below, except to the extent that the context in which they are used indicates otherwise. The following abbreviations and terms have the indicated meanings throughout:

The terms “compound of Formula I” and “compound of Formula II” are intended to include any stereoisomer, tautomer, and/or pharmaceutically acceptable salt as defined herein. Compounds of Formula I and Formula II also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form,” “polymorph,” and “novel form” may be used interchangeably herein, and are meant to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to. Compounds of Formula I and compounds of Formula II also include pharmaceutically acceptable forms of the recited compounds, including chelates, non-covalent complexes, prodrugs, and mixtures thereof.

As noted above, prodrugs also fall within the scope of compounds of Formula I and compounds of Formula II. In some embodiments, the “prodrugs” described herein include any compound that becomes a compound of Formula I and/or Formula II when administered to a patient, for example, upon metabolic processing of the prodrug. Examples of prodrugs include derivatives of functional groups, such as a carboxylic acid group, in the compounds of Formula I and/or Formula II. Exemplary prodrugs of a carboxylic acid group include, but are not limited to, carboxylic acid esters such as alkyl esters, hydroxyalkyl esters, arylalkyl esters, and aryloxyalkyl esters.

A “solvate” is formed by the interaction of a solvent and a compound. The terms “compound of Formula I” and “compounds of Formula II” are intended to include solvates of compounds. Similarly, “salts” includes solvates of salts. Suitable solvates are pharmaceutically acceptable solvates, such as hydrates, including monohydrates and hemi-hydrates.

As used herein, the term “hydrate” refers to a crystal form with either a stoichiometric or non-stoichiometric amount of water is incorporated into the crystal structure.

A “chelate” is formed by the coordination of a compound to a metal ion at two (or more) points. The term “compound” is intended to include chelates of compounds. Similarly, “salts” includes chelates of salts.

A “non-covalent complex” is formed by the interaction of a compound and another molecule wherein a covalent bond is not formed between the compound and the molecule. For example, complexation can occur through van der Waals interactions, hydrogen bonding, and electrostatic interactions (also called ionic bonding). Such non-covalent complexes are included in the term “compound’.

“Subject” refers to an animal, such as a mammal, that has been or will be the object of treatment, observation, or experiment. The methods described herein may be useful for both human therapy and veterinary applications. In one embodiment, the subject is a human.

As used herein, “treatment” or “treating” refers to an amelioration of a disease or disorder, or at least one discernible symptom thereof. In another embodiment, “treatment” or “treating” refers to an amelioration of at least one measurable physical parameter, not necessarily discernible by the patient. In yet another embodiment, “treatment” or “treating” refers to reducing the progression of a disease or disorder, either physically, for example, stabilization of a discernible symptom, physiologically, for example, stabilization of a physical parameter, or both. In yet another embodiment, “treatment” or “treating” refers to delaying the onset of a disease or disorder.

As used herein, “prevention” or “preventing” refers to a reduction of the risk of acquiring a given disease or disorder.

A dash (“-”) that is not between two letters or symbols is used to indicate a point of attachment for a substituent. For example, —CONH₂ is attached through the carbon atom.

By “optional” or “optionally” is meant that the subsequently described event or circumstance may or may not occur, and that the description includes instances where the event or circumstance occurs and instances in which is does not. For example, “optionally substituted aryl” encompasses both “aryl” and “substituted aryl” as defined below. It will be understood by those skilled in the art, with respect to any group containing one or more substituents, that such groups are not intended to introduce any substitution or substitution patterns that are sterically impractical, synthetically non-feasible and/or inherently unstable.

The term “acyl” term as used herein refers to a carbonyl radical attached to an alkyl, alkenyl, alkynyl, cycloalkyl, heterocycyl, aryl, or heteroaryl. Exemplary acyl groups include, but are not limited to, acetyl, formyl, propionyl, benzoyl, and the like.

The term “aldehyde” or “formyl” as used herein refers to —CHO.

The term “alkenyl” as used herein refers to an unsaturated straight or branched hydrocarbon having at least one carbon-carbon double bond, such as a straight or branched group of 2-22, 2-8, or 2-6 carbon atoms, referred to herein as (C₂-C₂₂)alkenyl, (C₂-C₈)alkenyl, and (C₂-C₆)alkenyl, respectively. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, and 4-(2-methyl-3-butene)-pentenyl.

The term “alkoxy” as used herein refers to an alkyl group attached to an oxygen (—O-alkyl-). “Alkoxy” groups also include an alkenyl group attached to an oxygen (“alkenyloxy”) or an alkynyl group attached to an oxygen (“alkynyloxy”) groups. Exemplary alkoxy groups include, but are not limited to, groups with an alkyl, alkenyl or alkynyl group of 1-22, 1-8, or 1-6 carbon atoms, referred to herein as (C₁-C₂₂)alkoxy, (C₁-C₈)alkoxy, and (C₁-C₆)alkoxy, respectively. Exemplary alkoxy groups include, but are not limited to methoxy and ethoxy.

The term “alkyl” as used herein refers to a saturated straight or branched hydrocarbon, such as a straight or branched group of 1-22, 1-8, or 1-6 carbon atoms, referred to herein as (C₁-C₂₂)alkyl, (C₁-C₈)alkyl, and (C₁-C₆)alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, and octyl.

The term “alkynyl” as used herein refers to an unsaturated straight or branched hydrocarbon having at least one carbon-carbon triple bond, such as a straight or branched group of 2-22, 2-8, or 2-6 carbon atoms, referred to herein as (C₂-C₂₂)alkynyl, (C₂-C₈)alkynyl, and (C₂-C₆)alkynyl, respectively. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl.

The term “amide” as used herein refers to the form —NRₐC(O)(R_{b})— or —C(O)NR_{b}R_{c}, wherein Rₐ, R_{b} and R_{c} are each independently selected from alkyl, alkenyl, alkynyl, aryl, arylalkyl, cycloalkyl, haloalkyl, heteroaryl, heterocyclyl, and hydrogen. The amide can be attached to another group through the carbon, the nitrogen, R_{b}, or R_{c}. The amide also may be cyclic, for example R_{b} and R_{c}, may be joined to form a 3- to 12-membered ring, such as a 3- to 10-membered ring or a 5- or 6-membered ring. The term “amide” encompasses groups such as sulfonamide, urea, ureido, carbamate, carbamic acid, and cyclic versions thereof. The term “amide” also encompasses an amide group attached to a carboxy group, for example, -amide-COON or salts such as -amide-COONa, an amino group attached to a carboxy group (for example, -amino-COON or salts such as -amino-COONa).

The term “amine” or “amino” as used herein refers to the form —NR_{d}Rₑ or —N(R_{d})Rₑ—, where R_{d} and Rₑ are independently selected from alkyl, alkenyl, alkynyl, aryl, arylalkyl, carbamate, cycloalkyl, haloalkyl, heteroaryl, heterocyclyl, and hydrogen. The amino can be attached to the parent molecular group through the nitrogen. The amino also may be cyclic, for example any two of R_{d} and Rₑ may be joined together or with the N to form a 3- to 12-membered ring (for example, morpholino or piperidinyl). The term amino also includes the corresponding quaternary ammonium salt of any amino group. Exemplary amino groups include alkylamino groups, wherein at least one of R_{d} or Rₑ is an alkyl group.

The term “aryl” as used herein refers to a mono-, bi-, or other multi-carbocyclic, aromatic ring system. The aryl group can optionally be fused to one or more rings selected from aryls, cycloalkyls, and heterocyclyls. The aryl groups of this invention can be substituted with groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Exemplary aryl groups also include, but are not limited to a monocyclic aromatic ring system, wherein the ring comprises 6 carbon atoms, referred to herein as “(C₆)aryl.”

The term “arylalkyl” as used herein refers to an alkyl group having at least one aryl substituent (for example, -aryl-alkyl-). Exemplary arylalkyl groups include, but are not limited to, arylalkyls having a monocyclic aromatic ring system, wherein the ring comprises 6 carbon atoms, referred to herein as “(C₆)arylalkyl.”

The term “aryloxy” as used herein refers to an aryl group attached to an oxygen atom. Exemplary aryloxy groups include, but are not limited to, aryloxys having a monocyclic aromatic ring system, wherein the ring comprises 6 carbon atoms, referred to herein as “(C₆)aryloxy.”

The term “arylthio” as used herein refers to an aryl group attached to an sulfur atom. Exemplary arylthio groups include, but are not limited to, arylthios having a monocyclic aromatic ring system, wherein the ring comprises 6 carbon atoms, referred to herein as “(C₆)arylthio.”

The term “arylsulfonyl” as used herein refers to an aryl group attached to a sulfonyl group, for example, —S(O)₂-aryl-. Exemplary arylsulfonyl groups include, but are not limited to, arylsulfonyls having a monocyclic aromatic ring system, wherein the ring comprises 6 carbon atoms, referred to herein as “(C₆)arylsulfonyl.”

The term “benzyl” as used herein refers to the group —CH₂-phenyl.

The term “bicyclic aryl” as used herein refers to an aryl group fused to another aromatic or non-aromatic carbocylic or heterocyclic ring. Exemplary bicyclic aryl groups include, but are not limited to, naphthyl or partly reduced forms thereof, such as di-, tetra-, or hexahydronaphthyl.

The term “bicyclic heteroaryl” as used herein refers to a heteroaryl group fused to another aromatic or non-aromatic carbocylic or heterocyclic ring. Exemplary bicyclic heteroaryls include, but are not limited to 5,6- or 6,6-fused systems, wherein one or both rings contain heteroatoms. The term “bicyclic heteroaryl” also encompasses reduced or partly reduced forms of fused aromatic system wherein one or both rings contain ring heteroatoms. The ring system may contain up to three heteroatoms, independently selected from oxygen, nitrogen, and sulfur. The bicyclic system may be optionally substituted with one or more groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Exemplary bicyclic heteroaryl's include, but are not limited to, quinazolinyl, benzothiophenyl, benzoxazolyl, benzimidazolyl, benzothiazolyl, benzofuranyl, indolyl, quinolinyl, isoquinolinyl, phthalazinyl, benzotriazolyl, benzopyridinyl, and benzofuranyl.

The term “carbamate” as used herein refers to the form —R_{g}OC(O)N(Rₕ)—, —R_{g}OC(O)N(Rₕ)Rᵢ—, or —OC(O)NRₕRᵢ, wherein R_{g}, Rₕ and Rᵢ are each independently selected from alkyl, alkenyl, alkynyl, aryl, arylalkyl, cycloalkyl, haloalkyl, heteroaryl, heterocyclyl, and hydrogen. Exemplary carbamates include, but are not limited to, arylcarbamates or heteroaryl carbamates (for example, wherein at least one of R_{g}, Rₕ and Rᵢ are independently selected from aryl or heteroaryl, such as pyridine, pyridazine, pyrimidine, and pyrazine).

The term “carbonyl” as used herein refers to —C(O)—.

The term “carboxy” as used herein refers to —COON or its corresponding carboxylate salts (for example, —COONa). The term carboxy also includes “carboxycarbonyl,” for example a carboxy group attached to a carbonyl group, for example, —C(O)—COON or salts, such as —C(O)—COONa.

The term “cyano” as used herein refers to —CN.

The term “cycloalkoxy” as used herein refers to a cycloalkyl group attached to an oxygen.

The term “cycloalkyl” as used herein refers to a saturated or unsaturated cyclic, bicyclic, or bridged bicyclic hydrocarbon group of 3-12 carbons, or 3-8 carbons, referred to herein as “(C₃-C₈)cycloalkyl,” derived from a cycloalkane. Exemplary cycloalkyl groups include, but are not limited to, cyclohexanes, cyclohexenes, cyclopentanes, and cyclopentenes. Cycloalkyl groups may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Cycloalkyl groups can be fused to other cycloalkyl saturated or unsaturated, aryl, or heterocyclyl groups.

The term “dicarboxylic acid” as used herein refers to a group containing at least two carboxylic acid groups such as saturated and unsaturated hydrocarbon dicarboxylic acids and salts thereof. Exemplary dicarboxylic acids include alkyl dicarboxylic acids. Dicarboxylic acids may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydrogen, hydroxyl, ketone, nitro, phosphate, sulfide, sulfinyl, sulfonyl, sulfonic acid, sulfonamide and thioketone. Dicarboxylic acids include, but are not limited to succinic acid, glutaric acid, adipic acid, suberic acid, sebacic acid, azelaic acid, maleic acid, phthalic acid, aspartic acid, glutamic acid, malonic acid, fumaric acid, (+)/(−)-malic acid, (+)/(−) tartaric acid, isophthalic acid, and terephthalic acid. Dicarboxylic acids further include carboxylic acid derivatives thereof, such as anhydrides, imides, hydrazides (for example, succinic anhydride and succinimide).

The term “ester” refers to the structure —C(O)O—, —C(O)O—Rᵢ—, —RₖC(O)O—Rⱼ—, or —RₖC(O)O—, where 0 is not bound to hydrogen, and Rⱼ and Rₖ can independently be selected from alkoxy, aryloxy, alkyl, alkenyl, alkynyl, amide, amino, aryl, arylalkyl, cycloalkyl, ether, haloalkyl, heteroaryl, and heterocyclyl. Rₖ can be a hydrogen, but Rⱼ cannot be hydrogen. The ester may be cyclic, for example the carbon atom and Rⱼ, the oxygen atom and Rₖ, or Rᵢ and Rₖ may be joined to form a 3- to 12-membered ring. Exemplary esters include, but are not limited to, alkyl esters wherein at least one of Rⱼ or Rₖ is alkyl, such as —O—C(O)-alkyl, —C(O)—O-alkyl-, and -alkyl-C(O)—O-alkyl-. Exemplary esters also include aryl or heteoraryl esters, for example wherein at least one of Rⱼ or Rₖ is a heteroaryl group such as pyridine, pyridazine, pyrmidine and pyrazine, such as a nicotinate ester. Exemplary esters also include reverse esters having the structure —RₖC(O)O—, where the oxygen is bound to the parent molecule. Exemplary reverse esters include succinate, D-argininate, L-argininate, L-lysinate and D-lysinate. Esters also include carboxylic acid anhydrides and acid halides.

The term “ether” refers to the structure —RₗO—Rₘ—, where Rₗ and Rₘ can independently be alkyl, alkenyl, alkynyl, aryl, cycloalkyl, heterocyclyl, and ether. The ether can be attached to the parent molecular group through Rₗ or Rₘ. Exemplary ethers include, but are not limited to, alkoxyalkyl and alkoxyaryl groups. Ethers also includes polyethers, for example, where one or both of Rₗ and Rₘ are ethers.

The terms “halo” or “halogen” or “Hal” as used herein refer to F, Cl, Br, or I.

The term “haloalkyl” as used herein refers to an alkyl group substituted with one or more halogen atoms. “Haloalkyls” also encompass alkenyl or alkynyl groups substituted with one or more halogen atoms.

The term “heteroaryl” as used herein refers to a mono-, bi-, or multi-cyclic, aromatic ring system containing one or more heteroatoms, for example 1-3 heteroatoms, such as nitrogen, oxygen, and sulfur. Heteroaryls can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heteroaryls can also be fused to non-aromatic rings. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidilyl, tetrazolyl, furyl, thienyl, isoxazolyl, thiazolyl, furyl, phenyl, isoxazolyl, and oxazolyl. Exemplary heteroaryl groups include, but are not limited to, a monocyclic aromatic ring, wherein the ring comprises 2-5 carbon atoms and 1-3 heteroatoms, referred to herein as “(C₂-C₅)heteroaryl.”

The terms “heterocycle,” “heterocyclyl,” or “heterocyclic” as used herein refer to a saturated or unsaturated 3-, 4-, 5-, 6- or 7-membered ring containing one, two, or three heteroatoms independently selected from nitrogen, oxygen, and sulfur. Heterocycles can be aromatic (heteroaryls) or non-aromatic. Heterocycles can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heterocycles also include bicyclic, tricyclic, and tetracyclic groups in which any of the above heterocyclic rings is fused to one or two rings independently selected from aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl.

The terms “hydroxy” and “hydroxyl” as used herein refers to —OH.

The term “hydroxyalkyl” as used herein refers to a hydroxy attached to an alkyl group.

The term “hydroxyaryl” as used herein refers to a hydroxy attached to an aryl group.

The term “ketone” as used herein refers to the structure —C(O)—Rn (such as acetyl, —C(O)CH₃ or —Rₙ—C(O)—Rₒ—. The ketone can be attached to another group through Rₙ or Rₒ. Rₙ or Rₒ can be alkyl, alkenyl, alkynyl, cycloalkyl, heterocyclyl or aryl, or Rₙ or Rₒ can be joined to form a 3- to 12-membered ring.

The term “monoester” as used herein refers to an analogue of a dicarboxylic acid wherein one of the carboxylic acids is functionalized as an ester and the other carboxylic acid is a free carboxylic acid or salt of a carboxylic acid. Examples of monoesters include, but are not limited to, to monoesters of succinic acid, glutaric acid, adipic acid, suberic acid, sebacic acid, azelaic acid, oxalic and maleic acid.

The term “nitro” as used herein refers to —NO₂.

The term “perfluoroalkoxy” as used herein refers to an alkoxy group in which all of the hydrogen atoms have been replaced by fluorine atoms.

The term “perfluoroalkyl” as used herein refers to an alkyl group in which all of the hydrogen atoms have been replaced by fluorine atoms. Exemplary perfluoroalkyl groups include, but are not limited to, C₁-C₅ perfluoroalkyl, such as trifluoromethyl.

The term “perfluorocycloalkyl” as used herein refers to a cycloalkyl group in which all of the hydrogen atoms have been replaced by fluorine atoms.

The term “phenyl” as used herein refers to a 6-membered carbocyclic aromatic ring. The phenyl group can also be fused to a cyclohexane or cyclopentane ring. Phenyl can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone.

The term “phosphate” as used herein refers to the structure —OP(O)O₂—, —RₓOP(O)O₂—, —OP(O)O₂R_{y}—, or —RₓOP(O)O₂R_{y}—, wherein Rₓ and R_{y} can be alkyl, alkenyl, alkynyl, aryl, cycloalkyl, heterocyclyl, hydrogen

The term “sulfide” as used herein refers to the structure —R_{z}S—, where R_{z} can be alkyl, alkenyl, alkynyl, aryl, arylalkyl, cycloalkyl, haloalkyl, heteroaryl, heterocyclyl. The sulfide may be cyclic, forming a 3 to 12-membered ring. The term “alkylsulfide” as used herein refers to an alkyl group attached to a sulfur atom.

The term “sulfinyl” as used herein refers to the structure —S(O)O—, —RₚS(O)O—, —RₚS(O)OR_{q}—, or —S(O)OR_{q}—, wherein Rₚ and R_{q} can be alkyl, alkenyl, aryl, arylalkyl, cycloalkyl, haloalkyl, heteroaryl, heterocyclyl, hydroxyl. Exemplary sulfinyl groups include, but are not limited to, alkylsulfinyls wherein at least one of Rₚ or R_{q} is alkyl, alkenyl, or alkynyl.

The term “sulfonamide” as used herein refers to the structure —(Rᵣ)—N—S(O)₂—Rₛ— or —Rₜ(Rᵣ)—N—S(O)₂—Rₛ, where Rₜ, Rᵣ, and Rₛ can be, for example, hydrogen, alkyl, alkenyl, alkynyl, aryl, cycloalkyl, and heterocyclyl. Exemplary sulfonamides include alkylsulfonamides (for example, where Rₛ is alkyl), arylsulfonamides (for example, where Rₛ is aryl), cycloalkyl sulfonamides (for example, where Rₛ is cycloalkyl), and heterocyclyl sulfonamides (for example, where Rₛ is heterocyclyl).

The term “sulfonate” as used herein refers to −OSO₃—. Sulfonate includes salts such as —OSO₃Na, —OSO₃K and the acid —OSO₃H.

The term “sulfonic acid” refers to —SO₃H— and its corresponding salts (for example, —SO₃K— and —SO₃Na—).

The term “sulfonyl” as used herein refers to the structure RᵤSO₂—, where Rᵤ can be alkyl, alkenyl, alkynyl, aryl, cycloalkyl, and heterocyclyl (for example, alkylsulfonyl). The term “alkylsulfonyl” as used herein refers to an alkyl group attached to a sulfonyl group. “Alkylsulfonyl” groups can optionally contain alkenyl or alkynyl groups.

The term “thioketone” refers to the structure —Rᵥ—C(S)—R_{w}—. The ketone can be attached to another group through Rᵥ or R_{w}. Rᵥ or R_{w} can be alkyl, alkenyl, alkynyl, cycloalkyl, heterocyclyl or aryl, or Rᵥ or R_{w} can be joined to form a 3- to 12-membered ring.

“Alkyl” groups can be substituted with or interrupted by or branched with at least one group selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, ketone, heteroaryl, heterocyclyl, hydroxyl, nitro, phosphate, sulfide, sulfinyl, sulfonyl, sulfonic acid, sulfonamide, thioketone, ureido and N. The substituents may be branched to form a substituted or unsubstituted heterocycle or cycloalkyl.

“Alkenyl,” “alkynyl”, “alkoxy”, “amino” and “amide” groups can be substituted with or interrupted by or branched with at least one group selected from alkoxy, aryloxy, alkyl, alkenyl, alkynyl, amide, amino, aryl, arylalkyl, carbamate, carbonyl, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, thioketone, ureido and N. The substituents may be branched to form a substituted or unsubstituted heterocycle or cycloalkyl.

As used herein, a “suitable substituent” refers to a group that does not nullify the synthetic or pharmaceutical utility of the compounds of the invention or the intermediates useful for preparing them. Examples of suitable substituents include, but are not limited to: C₁₋₂₂, C₁₋₈, and C₁₋₆ alkyl, alkenyl or alkynyl; C₁₋₆ aryl, C₂₋₅ heteroaryl; C₃-C₇ cycloalkyl; C₁-C₂₂, C₁₋₈, and C₁₋₆ alkoxy; C₆ aryloxy; —CN; —OH; oxo; halo, carboxy; amino, such as —NH(C₁₋₂₂, C₁₋₈, or C₁₋₆ alkyl), —N(C₁₋₂₂, C₁₋₈, and C₁₋₆ alkyl)₂, —NH((C₆)aryl), or —N((C₆)aryl)₂; formyl; ketones, such as —CO(C₁₋₂₂, C₁₋₈, and C₁₋₆ alkyl), —CO((C₆ aryl)esters, such as —CO₂(C₁₋₂₂, C₁₋₈, and C₁₋₆ alkyl) and —CO₂ (C₆ aryl). One of skill in art can readily choose a suitable substituent based on the stability and pharmacological and synthetic activity of the compound of the invention.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutically acceptable composition” as used herein refers to a composition comprising at least one compound as disclosed herein formulated together with one or more pharmaceutically acceptable carriers.

The term “pharmaceutically acceptable prodrugs” as used herein represents those prodrugs of the compounds of the present invention that are, within the scope of sound medical judgment, suitable for use in contact with the tissues of humans and lower animals without undue toxicity, irritation, allergic response, commensurate with a reasonable benefit/risk ratio, and effective for their intended use, as well as the zwitterionic forms, where possible, of the compounds of the invention. A discussion is provided in Higuchi et al., “Prodrugs as Novel Delivery Systems,” ACS Symposium Series, Vol. 14, and in Roche, E. B., ed. Bioreversible Carriers in Drug Design, American Pharmaceutical Association and Pergamon Press, 1987, both of which are incorporated herein by reference.

The term “pharmaceutically acceptable salt(s)” refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, matate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

In addition, if the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well known asymmetric synthetic methods.

Geometric isomers can also exist in the compounds of the present invention. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the E and Z isomers.

Substituents around a carbon-carbon double bond alternatively can be referred to as “cis” or “trans,” where “cis” represents substituents on the same side of the double bond and “trans” represents substituents on opposite sides of the double bond. The arrangements of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The compounds disclosed herein may exist as tautomers and both tautomeric forms are intended to be emcompassed by the scope of the invention, even though only one tautomeric structure is depicted. For example, any claim to compound A below is understood to include tautomeric structure B, and vice versa, as well as mixtures thereof.

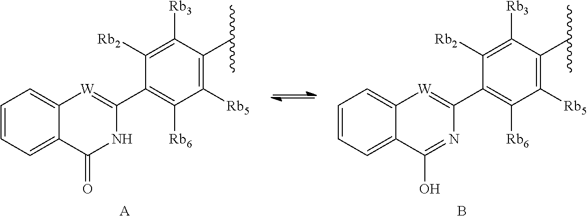

### EXEMPLARY EMBODIMENTS

### Embodiments Employing Compounds of Formula I

In certain embodiments, the method of inhibiting BET proteins comprises administering a therapeutically effective amount of at least one compound of Formula I or a stereoisomer, tautomer, pharmaceutically acceptable salt, or hydrate thereof, wherein:

### Q is CRa₃;

X is selected from OH, NH₂, S(O)₂NH₂, NHAc, and NHSO₂Me;
Ra₁ is selected from C₁-C₆ alkoxy;
Ra₂ is selected from hydrogen, C₁-C₆ alkoxy, amino, amide, and C₁-C₆ alkyl;
Ra₃ and Ra₄ are independently selected from hydrogen and C₁-C₆ alkoxy;
Rb₃ and Rb₅ are independently selected from C₁-C₆ alkyl and halogen.

In some embodiments, the method of inhibiting BET proteins comprises administering a therapeutically effective amount of at least one compound of Formula I, wherein:

Q is CRa₃;

Ra₃ is selected from hydrogen, methoxy,

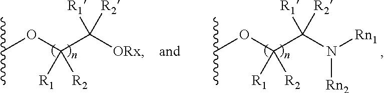

wherein:

n is 0, 1, 2, or 3;

R₁, R₁′, R₂, and R₂′ are independently selected from hydrogen, C₁-C₃ alkyl, cyclopropyl, and halogen wherein if n is 1, then R₂ and R₂′, R₁ and R₁′, R₁ and R₂′, or R₂ and R₁′ may form a double bond, wherein said double bond can be cis, trans, or a mixture thereof;

Rx is selected from C₁-C₆ alkyl, C₃-C₆ cycloalkyl, and aryl; and

Rn₁ and Rn₂ are independently selected from C₁-C₆ alkyl, C₃-C₆ cycloalkyl, and aryl.

In some embodiments, the method of inhibiting BET proteins comprises administering a therapeutically effective amount of at least one compound of Formula I, wherein:

Ra₃ is selected from hydrogen, methoxy,

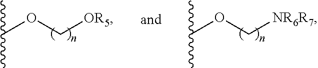

wherein

n is 1, 2, or 3;

R₅ is selected from C₁-C₆ alkyl substituted with one or more groups selected from methyl, phenyl, and pyridinyl; and

R₆ and R₇ are independently selected from unsubstituted C₁-C₆ alkyl.

In some embodiments, the method of inhibiting BET proteins comprises administering a therapeutically effective amount of at least one compound of Formula I, wherein:

Ra₃ is selected from hydrogen, methoxy, 2-methoxy-ethoxy, 2-dimethylamino-ethoxy, 2-benzyloxy-ethoxy, and 2-(pyridin-3-ylmethoxy)ethoxy.

In some embodiments, the method of inhibiting BET proteins comprises administering a therapeutically effective amount of at least one compound of Formula I, wherein:

Ra₄ is selected from hydrogen and unsubstituted C₁-C₆ alkoxy.

In some embodiments, the method of inhibiting BET proteins comprises administering a therapeutically effective amount of at least one compound of Formula I, wherein:

Ra₄ is selected from hydrogen and methoxy.

In some embodiments, the method of inhibiting BET proteins comprises administering a therapeutically effective amount of at least one compound of Formula I, wherein:

X is OH.

In some embodiments, the method of inhibiting BET proteins comprises administering a therapeutically effective amount of at least one compound of Formula I, wherein:

Ra₁ is selected from methoxy,

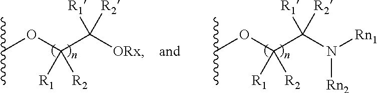

wherein:

n is 0, 1, 2, or 3;

R₁, R₁′, R₂, and R₂′ are independently selected from hydrogen, C₁-C₃ alkyl, cyclopropyl, and halogen wherein if n is 1, then R₂ and R₂′, R₁ and R₁′, R₁ and R₂′, or R₂ and R₁′ may form a double bond, wherein said double bond can be cis, trans, or a mixture thereof;

Rx is selected from C₁-C₆ alkyl, C₃-C₆ cycloalkyl, and aryl; and

Rn₁ and Rn₂ are independently selected from C₁-C₆ alkyl, C₃-C₆ cycloalkyl, and aryl.

In some embodiments, the method of inhibiting BET proteins comprises administering a therapeutically effective amount of at least one compound of Formula I, wherein:

Ra₁ is selected from methoxy,

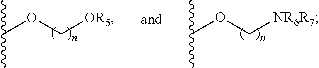

n is 1, 2, or 3; and

R₅, R₆, and R₇ are independently selected from unsubstituted C₁-C₆ alkyl.

In some embodiments, the method of inhibiting BET proteins comprises administering a therapeutically effective amount of at least one compound of Formula I, wherein:

Ra₁ is selected from hydrogen, methoxy, 2-methoxy-ethoxy, and 2-dimethylamino-ethoxy.

In some embodiments, the method of inhibiting BET proteins comprises administering a therapeutically effective amount of at least one compound of Formula I, wherein:

Ra₂ is selected from hydrogen, unsubstituted C₁-C₆ alkoxy, NHR₉, and C₁-C₆ alkyl substituted with heterocycle or amino; and

R₉ is selected from acyl, and heteroaryl.

In some embodiments, the method of inhibiting BET proteins comprises administering a therapeutically effective amount of at least one compound of Formula I, wherein:

Ra₂ is selected from hydrogen, methoxy, acetamido, morpholin-4-ylmethyl, pyridin-2-ylamino, (4-methylpiperazin-1-yl)methyl, and methanesulfonamido.

In some embodiments, the method of inhibiting BET proteins comprises administering a therapeutically effective amount of at least one compound of Formula I, wherein:

Rb₃ and Rb₅ are independently selected from unsubtituted C₁-C₆ alkyl and halogen.

In some embodiments, the method of inhibiting BET proteins comprises administering a therapeutically effective amount of at least one compound of Formula I, wherein:

Rb₃ and Rb₅ are independently selected from methyl, tert-butyl, fluorine, and chlorine.

In certain embodiments, the method of inhibiting BET proteins comprises administering a therapeutically effective amount of at least one compound selected from:
3-(3-fluoro-4-hydroxyphenyl)-5-methoxyisoquinolin-1(2H)-one;
3-(4-hydroxy-3,5-dimethylphenyl)-6,8-dimethoxyisoquinolin-1(2H)-one;
2-(4-hydroxy-3,5-dimethylphenyl)-5,7-dimethoxyquinazolin-4(3H)-one;
7-(4-hydroxy-3,5-dimethylphenyl)-2,4-dimethoxy-1,6-naphthyridin-5(6H)-one;
2-(3,5-di-tert-butyl-4-hydroxyphenyl)-5,7-dimethoxyquinazolin-4(3H)-one;
2-(3-chloro-4-hydroxyphenyl)-5,7-dimethoxyquinazolin-4(3H)-one;
2-(4-hydroxy-3,5-dimethylphenyl)-6,7-dimethoxyquinazolin-4(3H)-one;
N-(2-(4-hydroxy-3,5-dimethylphenyl)-4-oxo-3,4-dihydroquinazol in-6-yl)acetamide;
2-(4-hydroxy-3,5-dimethylphenyl)-6-(morpholinomethyl)quinazolin-4(3H)-one;
2-(4-hydroxy-3,5-dimethylphenyl)-5,7-dimethoxypyrido[2,3-d]pyrimidin-4(3H)-one;
2-(4-hydroxy-3,5-dimethylphenyl)-5,7-dimethoxy-6-(morpholinomethyl)quinazolin-4(3H)-one;
5-(2-dimethylamino-ethoxy)-2(4-hydroxy-3,5-dimethylphenyl)-7-methoxy-3H-quinazolin-4-one;
2-(4-hydroxy-3,5-dimethyl-phenyl)-7-methoxy-5-(2-methoxy-ethoxy)-3H-quinazolin-4-one;
7-(2-amino-ethoxy)-2-(4-hydroxy-3,5-dimethyl-phenyl)-5-methoxy-3H-quinazolin-4-one;
2-(4-hydroxy-3,5-dimethyl-phenyl)-5-methoxy-7-(2-methoxy-ethoxy)-3H-quinazolin-4-one;
7-(2-benzyloxy-ethoxy)-2-(4-hydroxy-3,5-dimethyl-phenyl)-5-methoxy-3H-quinazolin-4-one;
2-(4-hydroxy-3,5-dimethylphenyl)-5-methoxy-7-[2-(pyridin-3-ylmethoxy)ethoxy]-3H-quinazolin-4-one;
7-(2-dimethylamino-ethoxy)-2-(4-hydroxy-3,5-dimethylphenyl)-3H-quinazol in-4-one;
2-(4-hydroxy-3,5-dimethyl-phenyl)-6-(pyridin-4-ylamino)-3H-quinazol in-4-one;
2-(4-hydroxy-3,5-dimethyl-phenyl)-6-(pyridin-2-ylamino)-3H-quinazol in-4-one;
2-(4-hydroxy-3,5-dimethylphenyl)-6-(4-methylpiperazin-1-yl)methyl)quinazolin-4(3H)-one;
N-((2-(4-hydroxy-3,5-dimethylphenyl)-4-oxo-3,4-dihydroquinazolin-6-yl)methyl)methanesulfonamide; and
tatutomers, stereoisomers, pharmaceutically acceptable salts, and hydrates thereof.

In certain embodiments, the method of inhibiting BET proteins comprises administering a therapeutically effective amount of at least one compound selected from:
5-(2-dimethylamino-ethoxy)-2(4-hydroxy-3,5-dimethylphenyl)-7-methoxy-3H-quinazolin-4-one;
2-(4-hydroxy-3,5-dimethyl-phenyl)-7-methoxy-5-(2-methoxy-ethoxy)-3H-quinazolin-4-one;
7-(2-amino-ethoxy)-2-(4-hydroxy-3,5-dimethyl-phenyl)-5-methoxy-3H-quinazolin-4-one;
2-(4-hydroxy-3,5-dimethyl-phenyl)-5-methoxy-7-(2-methoxy-ethoxy)-3H-quinazolin-4-one;
7-(2-benzyloxy-ethoxy)-2-(4-hydroxy-3,5-dimethyl-phenyl)-5-methoxy-3H-quinazolin-4-one;
2-(4-hydroxy-3,5-dimethylphenyl)-5-methoxy-7-[2-(pyridin-3-ylmethoxy)ethoxy]-3H-quinazolin-4-one;
7-(2-dimethylamino-ethoxy)-2-(4-hydroxy-3,5-dimethylphenyl)-3H-quinazol in-4-one;
2-(4-hydroxy-3,5-dimethyl-phenyl)-6-(pyridin-4-ylamino)-3H-quinazol in-4-one;
2-(4-hydroxy-3,5-dimethyl-phenyl)-6-(pyridin-2-ylamino)-3H-quinazol in-4-one;
2-(4-hydroxy-3,5-dimethylphenyl)-6-(4-methylpiperazin-1-yl)methyl)quinazolin-4(3H)-one;
N-((2-(4-hydroxy-3,5-dimethylphenyl)-4-oxo-3,4-dihydroquinazolin-6-yl)methyl)methanesulfonamide; and
tautomers, stereoisomers, pharmaceutically acceptable salts, and hydrates thereof.

### Embodiments Employing Compounds of Formula II

In certain embodiments, the method of inhibiting BET proteins comprises administering a therapeutically effective amount of at least one compound of Formula II or a stereoisomer, tautomer, pharmaceutically acceptable salt, or hydrate thereof, wherein:

P is CRa₁;

Ra₁ is selected from C₁-C₆ alkyl, C₁-C₆ alkoxy, and halogen;

Ra₃ is independently selected from hydrogen, C₁-C₆ alkoxy, C₁-C₆ alkyl, and halogen.

In some embodiments, the method of inhibiting BET proteins comprises administering a therapeutically effective amount of at least one compound of Formula II, wherein:

Ra₁ is selected from unsubstituted C₁-C₆ alkyl and unsubstituted C₁-C₆ alkoxy.

In some embodiments, the method of inhibiting BET proteins comprises administering a therapeutically effective amount of at least one compound of Formula II, wherein:

Ra₁ is selected from methyl, methoxy, ethyl, and ethoxy.

In some embodiments, the method of inhibiting BET proteins comprises administering a therapeutically effective amount of at least one compound of Formula II, wherein:

Ra₃ is selected from selected from hydrogen, methoxy, unsubstituted C₁-C₆ alkyl, halogen, and

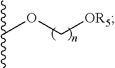

n is 1, 2, or 3; and

R₅ is C₁-C₆ alkyl substituted with phenyl or heteroaryl.

In some embodiments, the method of inhibiting BET proteins comprises administering a therapeutically effective amount of at least one compound of Formula II, wherein:

Ra₃ is selected from selected from hydrogen, methoxy, chlorine, fluorine, isopropoxy, methyl, 2-benzyloxy-ethoxy, and 2-(pyridin-3-ylmethoxy)ethoxy.

In some embodiments, the method of inhibiting BET proteins comprises administering a therapeutically effective amount of at least one compound of Formula II, wherein:

Rb₃ and Rb₅ are independently selected from hydrogen, methyl, C₁-C₆ alkyl substituted with heterocyclyl, and unsubstituted C₁-C₆ alkoxy.

In some embodiments, the method of inhibiting BET proteins comprises administering a therapeutically effective amount of at least one compound of Formula II, wherein:

Rb₃ and Rb₅ are independently selected from hydrogen, methyl, methoxy, and morpholinomethyl.

In some embodiments, the method of inhibiting BET proteins comprises administering a therapeutically effective amount of at least one compound of Formula II, wherein:

Rd is selected from C₁-C₆ alkoxy, C₃-C₆ cycloalkyl,

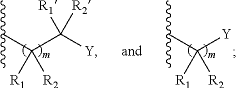

m is selected from 1, 2, or 3;

R₁, R₁′, R₂, and R₂′ are independently selected from hydrogen, fluorine, C₁-C₆ alkyl, hydroxyl, —NH₂, and C₁-C₆ alkoxy wherein R₂ and R₂′ may be eliminated to form a double bond;

Y is selected from OH, SH, NH₂, -Oalkyl, -Oaryl, —CH₂aryl, —C(O)NHalkyl, —C(O)N(alkyl)₂, —C(O)NHaryl, —NHacyl, —NHalkyl, —NHS(O)₂alkyl, —N(alkyl)₂, —NHS(O)₂N(alkyl)₂, —NHCN, and —NHC(O)N(alkyl)₂, —NHheterocyclyl, and heterocyclyl; and

Rd may be connected to Rb₃ or Rb₅ to form a heterocycle;

provided that for —N(alkyl)₂ the alkyl chains cannot be joined to form an aryl or heterocyclic ring.

In some embodiments, the method of inhibiting BET proteins comprises administering a therapeutically effective amount of at least one compound of Formula II, wherein:

Rd is connected to Rb₃ or Rb₅ to form a heterocycle selected from substituted furanyl or substituted pyrrolyl.

In some embodiments, the method of inhibiting BET proteins comprises administering a therapeutically effective amount of at least one compound of Formula II, wherein:

Rd is connected to Rb₃ or Rb₅ to form a heterocycle selected from 2-hydroxymethyl-furan-5-yl or 2-(4,5-dihydro-1H-pyrrol-2-yl)ethanol.

In some embodiments, the method of inhibiting BET proteins comprises administering a therapeutically effective amount of at least one compound of Formula II, wherein:

X-Rd is selected from 2-hydroxy-2-methylpropoxy, 2-hydroxyethoxy, methoxy, benzyloxyethoxy, 2,3-dihydroxypropoxy, aminocarbonylethoxy, methylaminocarbonylethoxy, (4-methoxyphenyl)aminocarbonylethoxy, benzylaminocarbonylethoxy, 4-hydroxybutoxy, (5-phenyl-4H-[1,2,4]triazol-3-ylamino)ethoxy, (3-methyl-[1,2,4]oxadiazol-5-ylamino)ethoxy, methylcarbonylaminoethoxy, methylcarbonylaminomethyl, (2,2,2-trifluoro-ethylamino)ethoxy, methanesulfonylaminoethoxy, isobutyrylaminoethoxy, methylaminoethoxy, isopropylsulfonylaminoethoxy, methylcarbonylaminoethoxy, dimethylaminoethoxy, N-(2-hydroxyethyl)-N-methylacetamide, formamide-N-2-ethoxy, methylformamide-N2-ethoxy, dimethylsulfonylaminoethoxy, cyanoaminoethoxy, (5-methylisoxazol-3-ylamino)ethoxy, (pyrimidin-2-ylamino)ethoxy, (isoxazol-3-ylamino)ethoxy, (4,6-dimethoxypyrimidin-2-ylamino)ethoxy, 3-hydroxypropyl, and 2-hydroxyethyl.

In some embodiments, the method of inhibiting BET proteins comprises administering a therapeutically effective amount of at least one compound of Formula II, wherein:

X-Rd is selected from hydroxyethoxy, methylcarbonylaminoethoxy, (4-methoxyphenyl)aminocarbonylethoxy, and isobutyrylaminoethoxy.

In certain embodiments, the method of inhibiting BET proteins comprises administering a therapeutically effective amount of at least one compound of Formula II, selected from:
3-(4-(2-hydroxy-2-methylpropoxy)-3,5-dimethylphenyl)-6,8-dimethoxyisoquinolin-1(2H)-one;
2-(4-(2-hydroxyethoxy)-3,5-dimethylphenyl)-5,7-dimethoxyquinazolin-4(3H)-one;
5,7-dimethoxy-2-(4-methoxyphenyl)quinazol in-4(3H)-one;
2-(4-(2-hydroxyethoxy)-3,5-dimethylphenyl)-6,7-dimethoxyquinazolin-4(3H)-one;
5,7-dimethoxy-2-(4-methoxy-3-(morpholinomethyl)phenyl)quinazolin-4(3H)-one;
2-(4-(2-hydroxyethoxy)-3,5-dimethylphenyl)-5,7-dimethoxypyrido[2,3-d]pyrimidin-4(3H)-one;
N-(2-(4-(2-hydroxyethoxy)-3,5-dimethylphenyl)-4-oxo-3,4-dihydroquinazolin-6-yl)acetamide;
2-(4-(2-(benzyloxy)ethoxy)-3,5-dimethylphenyl)-5,7-dimethoxypyrido[2,3-d]pyrimidin-4(3H)-one;
2-(4-(2-hydroxyethoxy)-3,5-dimethylphenyl)-5,7-dimethylpyrido[2,3-d]pyrimidin-4(3H)-one;
5,7-difluoro-2-(4-(2-hydroxyethoxy)-3,5-dimethylphenyl)quinazolin-4(3H)-one;
5,7-dichloro-2-(4-(2-hydroxyethoxy)-3,5-dimethylphenyl)quinazolin-4(3H)-one;
2-[4-(2-hydroxy-ethoxy)-3,5-dimethyl-phenyl]-5,7-diisopropoxy-3H-quinazol in-4-one;
2-[4-(2,3-Dihydroxy-propoxy)-3,5-dimethyl-phenyl]-5,7-dimethoxy-3H-quinazolin-4-one;
2-[4-(2-hydroxy-ethoxy)-phenyl]-5,7-dimethoxy-3H-quinazolin-4-one;
2-[4-(2-hydroxy-ethoxy)-naphthalen-1-yl]-5,7-dimethoxy-3H-quinazolin-4-one;
2-(2-hydroxymethyl-benzofuran-5-yl)-5,7-dimethoxy-3H-quinazolin-4-one;
7-(2-benzyloxy-ethoxy)-2-[4-(2-hydroxy-ethoxy)-3,5-dimethyl-phenyl]-5-methoxy-3H-quinazolin-4-one;
7-(2-benzyloxy-ethoxy)-2-(2-hydroxymethyl-benzofuran-5-yl)-5-methoxy-3H-quinazolin-4-one;
2-[4-(5,7-dimethoxy-4-oxo-3,4-dihydro-quinazolin-2-yl)-2,6-dimethyl-phenoxy]-acetamide;
2-[4-(5,7-dimethoxy-4-oxo-3,4-dihydro-quinazolin-2-yl)-2,6-dimethyl-phenoxy]-N-methyl-acetamide;
2-[4-(5,7-Dimethoxy-4-oxo-3,4-dihydro-quinazolin-2-yl)-2,6-dimethyl-phenoxy]-N-(4-methoxy-phenyl)-acetamide;
N-benzyl-2-[4-(5,7-dimethoxy-4-oxo-3,4-dihydroquinazolin-2-yl)-2,6-dimethylphenoxy]acetamide;
2-[4-(4-hydroxy-butoxy)-3,5-dimethyl-phenyl]-5,7-dimethoxy-3H-quinazolin-4-one;
2-(4-(2-hydroxyethoxy)-3,5-dimethylphenyl)-5-methoxyquinazolin-4(3H)-one;
7-chloro-2-(4-(2-hydroxyethoxy)-3,5-dimethylphenyl)quinazolin-4(3H)-one;
5-chloro-2-(4-(2-hydroxyethoxy)-3,5-dimethylphenyl)quinazolin-4(3H)-one;
2-(4-(2-hydroxyethoxy)-3,5-dimethylphenyl)-7-methoxyquinazolin-4(3H)-one;
5,7-dimethoxy-2-(4-methoxy-3,5-dimethylphenyl)quinazolin-4(3H)-one;
2-(4-(2-hydroxyethoxy)-3-methylphenyl)-5,7-dimethoxyquinazolin-4(3H)-one;
5,7-Dimethoxy-2-{3-methyl-4-[2-(5-phenyl-4H-[1,2,4]triazol-3-ylamino)-ethoxy]-phenyl}-3H-quinazolin-4-one;
2-{3,5-Dimethyl-4-[2-(3-methyl-[1,2,4]oxadiazol-5-ylamino)-ethoxy]-phenyl}-5,7-dimethoxy-3H-quinazolin-4-one;
N-{2-[4-(5,7-dimethoxy-4-oxo-3,4-dihydro-pyrido[2,3-d]pyrimidin-2-yl)-2,6-dimethyl-phenoxy]-ethyl}-acetamide;
N-(4-(5,7-dimethoxy-4-oxo-3,4-dihydroquinazolin-2-yl)-2,6-dimethylbenzyl)acetamide;
N-[4-(5,7-dimethoxy-4-oxo-3,4-dihydro-pyrido[2,3-d]pyrimidin-2-yl)-2,6-dimethyl-benzyl]-acetamide;
2-{3,5-Dimethyl-4-[2-(2,2,2-trifluoro-ethylamino)-ethoxy]-phenyl}-5,7-dimethoxy-3H-quinazolin-4-one;
N-{2-[4-(6,8-Dimethoxy-1-oxo-1,2-dihydro-isoquinolin-3-yl)-2,6-dimethyl-phenoxy]-ethyl}-formamide;
N-(2-(4-(5,7-dimethoxy-4-oxo-3,4-dihydroquinazolin-2-yl)-2,6-dimethylphenoxy)ethyl)methanesulfonamide;
N-(2-(4-(5,7-dimethoxy-4-oxo-3,4-dihydroquinazolin-2-yl)-2,6-dimethylphenoxy)ethyl)-4-methoxybenzamide;
N-(2-(4-(5,7-dimethoxy-4-oxo-3,4-dihydroquinazolin-2-yl)-2,6-dimethylphenoxy)ethyl)acetamide;
N-(2-(4-(5,7-dimethoxy-4-oxo-3,4-dihydroquinazolin-2-yl)-2,6-dimethylphenoxy)ethyl)isobutyramide;
2-(3,5-dimethyl-4-(2-(methylamino)ethoxy)phenyl)-5,7-dimethoxyquinazolin-4(3H)-one;
N-(2-(4-(5,7-dimethoxy-4-oxo-3,4-dihydroquinazolin-2-yl)-2,6-dimethylphenoxy)ethyl)propane-2-sulfonamide;
2-(4-(2-(isopropylamino)ethoxy)-3,5-dimethylphenyl)-5,7-dimethoxyquinazolin-4(3H)-one;
N-(2-(4-(5,7-dimethoxy-4-oxo-3,4-dihydroquinazolin-2-yl)-2-methylphenoxy)ethyl)acetamide;
N-(2-(4-(5,7-dimethoxy-4-oxo-3,4-dihydroquinazolin-2-yl)-2-methylphenoxy)ethyl)isobutyramide;
N-(2-(4-(5,7-dimethoxy-4-oxo-3,4-dihydroquinazolin-2-yl)-2-methylphenoxy)ethyl)methanesulfonamide;
2-(4-(2-(dimethylamino)ethoxy)-3,5-dimethylphenyl)-5,7-dimethoxyquinazolin-4(3H)-one;
N-(2-(4-(5,7-dimethoxy-4-oxo-3,4-dihydroquinazolin-2-yl)-2,6-dimethylphenoxy)ethyl)-N-methylacetamide;
N-(2-(4-(5,7-dimethoxy-4-oxo-3,4-dihydroquinazolin-2-yl)-2,6-dimethylphenoxy)ethyl)formamide;
N-(2-(4-(5,7-dimethoxy-4-oxo-3,4-dihydroquinazolin-2-yl)-2,6-dimethylphenoxy)ethyl)-N-methylformamide;
N-(2-(4-(5,7-Dimethoxy-4-oxo-3,4-dihydroquinazolin-2-yl)-2,6-dimethylphenoxy)ethyl)dimethylamino-N-sulfonamide;
N-(2-(4-(5,7-dimethoxy-4-oxo-3,4-dihydroquinazolin-2-yl)-2,6-dimethylphenoxy)ethyl)cyanamide; 2-(3,5-dimethyl-4-(2-(5-methylisoxazol-3-ylamino)ethoxy)phenyl)-5,7-dimethoxyquinazolin-4(3H)-one;
2-(3,5-dimethyl-4-(2-(pyrimidin-2-ylamino)ethoxy)phenyl)-5,7-dimethoxyquinazolin-4(3H)-one;
2-(4-(2-(isoxazol-3-ylamino)ethoxy)-3,5-dimethylphenyl)-5,7-dimethoxyquinazolin-4(3H)-one;
2-(4-(2-(4,6-dimethoxypyrimidin-2-ylamino)ethoxy)-3,5-dimethylphenyl)-5,7-dimethoxyquinazolin-4(3H)-one;
2-[4-(3-hydroxy-propyl)-3,5-dimethoxyphenyl]-5,7-dimethoxy-3H-quinazolin-4-one;
2-[4-(3-hydroxy-propyl)-3-methoxy-phenyl]-5,7-dimethoxy-3H-quinazolin-4-one;
2-[2-(2-hydroxyethyl)-1H-indol-6-yl]-5,7-dimethoxy-3H-quinazolin-4-one; and
tautomers, stereoisomers, pharmaceutically acceptable salts, and hydrates thereof.

Another aspect of the invention provides compounds of Formula II selected from:
2-(4-(2-(benzyloxy)ethoxy)-3,5-dimethylphenyl)-5,7-dimethoxypyrido[2,3-d]pyrimidin-4(3H)-one;
2-(4-(2-hydroxyethoxy)-3,5-dimethylphenyl)-5,7-dimethylpyrido[2,3-d]pyrimidin-4(3H)-one;
5,7-difluoro-2-(4-(2-hydroxyethoxy)-3,5-dimethylphenyl)quinazolin-4(3H)-one;
2-[4-(2-hydroxy-ethoxy)-3,5-dimethyl-phenyl]-5,7-diisopropoxy-3H-quinazol in-4-one;
2-[4-(2,3-Dihydroxy-propoxy)-3,5-dimethyl-phenyl]-5,7-dimethoxy-3H-quinazolin-4-one;
2-[4-(2-hydroxy-ethoxy)-phenyl]-5,7-dimethoxy-3H-quinazolin-4-one;
2-[4-(2-hydroxy-ethoxy)-naphthalen-1-yl]-5,7-dimethoxy-3H-quinazolin-4-one;
2-(2-hydroxymethyl-benzofuran-5-yl)-5,7-dimethoxy-3H-quinazolin-4-one;
7-(2-benzyloxy-ethoxy)-2-[4-(2-hydroxy-ethoxy)-3,5-dimethyl-phenyl]-5-methoxy-3H-quinazolin-4-one;
7-(2-benzyloxy-ethoxy)-2-(2-hydroxymethyl-benzofuran-5-yl)-5-methoxy-3H-quinazolin-4-one;
2-[4-(5,7-dimethoxy-4-oxo-3,4-dihydro-quinazolin-2-yl)-2,6-dimethyl-phenoxy]-N-methyl-acetamide;
2-[4-(5,7-Dimethoxy-4-oxo-3,4-dihydro-quinazolin-2-yl)-2,6-dimethyl-phenoxy]-N-(4-methoxy-phenyl)-acetamide;
N-benzyl-2-[4-(5,7-dimethoxy-4-oxo-3,4-dihydroquinazolin-2-yl)-2,6-dimethylphenoxy]acetamide;
2-[4-(4-hydroxy-butoxy)-3,5-dimethyl-phenyl]-5,7-dimethoxy-3H-quinazolin-4-one;
7-chloro-2-(4-(2-hydroxyethoxy)-3,5-dimethylphenyl)quinazolin-4(3H)-one;
5-chloro-2-(4-(2-hydroxyethoxy)-3,5-dimethylphenyl)quinazolin-4(3H)-one;
2-(4-(2-hydroxyethoxy)-3,5-dimethylphenyl)-7-methoxyquinazolin-4(3H)-one;
5,7-Dimethoxy-2-{3-methyl-4-[2-(5-phenyl-4H-[1,2,4]triazol-3-ylamino)-ethoxy]-phenyl}-3H-quinazolin-4-one;
2-{3,5-Dimethyl-4-[2-(3-methyl-[1,2,4]oxadiazol-5-ylamino)-ethoxy]-phenyl}-5,7-dimethoxy-3H-quinazolin-4-one;
N-{2-[4-(5,7-dimethoxy-4-oxo-3,4-dihydro-pyrido[2,3-d]pyrimidin-2-yl)-2,6-dimethyl-phenoxy]-ethyl}-acetamide;
N-(4-(5,7-dimethoxy-4-oxo-3,4-dihydroquinazolin-2-yl)-2,6-dimethylbenzyl)acetamide;
N-[4-(5,7-dimethoxy-4-oxo-3,4-dihydro-pyrido[2,3-d]pyrimidin-2-yl)-2,6-dimethyl-benzyl]-acetamide;
2-{3,5-Dimethyl-4-[2-(2,2,2-trifluoro-ethylamino)-ethoxy]-phenyl}-5,7-dimethoxy-3H-quinazolin-4-one;
N-{2-[4-(6,8-Dimethoxy-1-oxo-1,2-dihydro-isoquinolin-3-yl)-2,6-dimethyl-phenoxy]-ethyl}-formamide;
2-(3,5-dimethyl-4-(2-(methylamino)ethoxy)phenyl)-5,7-dimethoxyquinazolin-4(3H)-one;
N-(2-(4-(5,7-dimethoxy-4-oxo-3,4-dihydroquinazolin-2-yl)-2,6-dimethylphenoxy)ethyl)propane-2-sulfonamide;
2-(4-(2-(isopropylamino)ethoxy)-3,5-dimethylphenyl)-5,7-dimethoxyquinazolin-4(3H)-one;
N-(2-(4-(5,7-dimethoxy-4-oxo-3,4-dihydroquinazolin-2-yl)-2-methylphenoxy)ethyl)acetamide;
2-(4-(2-(dimethylamino)ethoxy)-3,5-dimethylphenyl)-5,7-dimethoxyquinazolin-4(3H)-one;
N-(2-(4-(5,7-dimethoxy-4-oxo-3,4-dihydroquinazolin-2-yl)-2,6-dimethylphenoxy)ethyl)-N-methylacetamide;
N-(2-(4-(5,7-dimethoxy-4-oxo-3,4-dihydroquinazolin-2-yl)-2,6-dimethylphenoxy)ethyl)formamide;
N-(2-(4-(5,7-dimethoxy-4-oxo-3,4-dihydroquinazolin-2-yl)-2,6-dimethylphenoxy)ethyl)-N-methylformamide;
N-(2-(4-(5,7-Dimethoxy-4-oxo-3,4-dihydroquinazolin-2-yl)-2,6-dimethylphenoxy)ethyl)dimethylamino-N-sulfonamide;
N-(2-(4-(5,7-dimethoxy-4-oxo-3,4-dihydroquinazolin-2-yl)-2,6-dimethylphenoxy)ethyl)cyanamide;
2-(3,5-dimethyl-4-(2-(5-methylisoxazol-3-ylamino)ethoxy)phenyl)-5,7-dimethoxyquinazolin-4(3H)-one;
2-(3,5-dimethyl-4-(2-(pyrimidin-2-ylamino)ethoxy)phenyl)-5,7-dimethoxyquinazolin-4(3H)-one;
2-(4-(2-(isoxazol-3-ylamino)ethoxy)-3,5-dimethylphenyl)-5,7-dimethoxyquinazolin-4(3H)-one;
2-(4-(2-(4,6-dimethoxypyrimidin-2-ylamino)ethoxy)-3,5-dimethylphenyl)-5,7-dimethoxyquinazolin-4(3H)-one;
2-[4-(3-hydroxy-propyl)-3,5-dimethoxyphenyl]-5,7-dimethoxy-3H-quinazolin-4-one;
2-[4-(3-hydroxy-propyl)-3-methoxy-phenyl]-5,7-dimethoxy-3H-quinazolin-4-one;
2-[2-(2-hydroxyethyl)-1H-indol-6-yl]-5,7-dimethoxy-3H-quinazolin-4-one; and
tautomers, stereoisomers, pharmaceutically acceptable salts, and hydrates thereof.

### Pharmaceutical Compositions

Pharmaceutical compositions comprising at least one compound of Formula I or II, or tautomer, stereoisomer, pharmaceutically acceptable salt or hydrate thereof formulated together with one or more pharmaceutically acceptable carriers may be employed in the methods of the invention. These formulations include those suitable for oral, rectal, topical, intraocular, buccal and parenteral (for example, subcutaneous, intramuscular, intradermal, intravenous, or via implants) administration. The most suitable form of administration in any given case will depend on the degree and severity of the condition being treated and on the nature of the particular compound being used.

Formulations suitable for oral administration may be presented in discrete units, such as capsules, cachets, lozenges, or tablets, each containing a predetermined amount of at least one compound of Formula I or II, or tautomer, stereoisomer, pharmaceutically acceptable salt or hydrate thereof as powder or granules; as a solution or a suspension in an aqueous or non-aqueous liquid; or as an oil-in-water or water-in-oil emulsion. As indicated, such formulations may be prepared by any suitable method of pharmacy which includes the step of bringing into association at least one compound of Formula I or II, or tautomer, stereoisomer, pharmaceutically acceptable salt or hydrate thereof as the active compound and a carrier or excipient (which may constitute one or more accessory ingredients). The carrier must be acceptable in the sense of being compatible with the other ingredients of the formulation and must not be deleterious to the recipient. The carrier may be a solid or a liquid, or both, and may be formulated with at least one compound described herein as the active compound in a unit-dose formulation, for example, a tablet, which may contain from about 0.05% to about 95% by weight of the at least one active compound. Other pharmacologically active substances may also be present including other compounds. The formulations employed in the methods of the invention may be prepared by any of the well known techniques of pharmacy consisting essentially of admixing the components.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by, for example, dissolving or dispersing, at least one active compound of Formula I or II, or tautomer, stereoisomer, pharmaceutically acceptable salt or hydrate thereof as described herein and optional pharmaceutical adjuvants in an excipient, such as, for example, water, saline, aqueous dextrose, glycerol, ethanol, and the like, to thereby form a solution or suspension. In general, suitable formulations may be prepared by uniformly and intimately admixing the at least one active compound of Formula I or II, or tautomer, stereoisomer, pharmaceutically acceptable salt or hydrate thereof with a liquid or finely divided solid carrier, or both, and then, if necessary, shaping the product. For example, a tablet may be prepared by compressing or molding a powder or granules of at least one compound of Formula I or II, or tautomer, stereoisomer, pharmaceutically acceptable salt or hydrate thereof, which may be optionally combined with one or more accessory ingredients. Compressed tablets may be prepared by compressing, in a suitable machine, at least one compound of the invention in a free-flowing form, such as a powder or granules, which may be optionally mixed with a binder, lubricant, inert diluent and/or surface active/dispersing agent(s). Molded tablets may be made by molding, in a suitable machine, where the powdered form of at least one compound of the invention is moistened with an inert liquid diluent.

Formulations suitable for buccal (sub-lingual) administration include lozenges comprising at least one compound of Formula I or II, or tautomer, stereoisomer, pharmaceutically acceptable salt or hydrate thereof in a flavored base, usually sucrose and acacia or tragacanth, and pastilles comprising the at least one compound in an inert base such as gelatin and glycerin or sucrose and acacia.

Formulations of the invention suitable for parenteral administration comprise sterile aqueous preparations of at least one compound of Formula I or II, or tautomer, stereoisomer, pharmaceutically acceptable salt, and hydrate thereof, which are approximately isotonic with the blood of the intended recipient. These preparations are administered intravenously, although administration may also be effected by means of subcutaneous, intramuscular, or intradermal injection. Such preparations may conveniently be prepared by admixing at least one compound described herein with water and rendering the resulting solution sterile and isotonic with the blood. Injectable compositions employed in the methods of the invention may contain from about 0.1 to about 5% w/w of the active compound.

Formulations suitable for rectal administration are presented as unit-dose suppositories. These may be prepared by admixing at least one compound as described herein with one or more conventional solid carriers, for example, cocoa butter, and then shaping the resulting mixture.

Formulations suitable for topical application to the skin may take the form of an ointment, cream, lotion, paste, gel, spray, aerosol, or oil. Carriers and excipients which may be used include Vaseline, lanoline, polyethylene glycols, alcohols, and combinations of two or more thereof. The active compound (i.e., at least one compound of Formula I or II, or tautomer, stereoisomer, pharmaceutically acceptable salt, and hydrate thereof) is generally present at a concentration of from about 0.1% to about 15% w/w of the composition, for example, from about 0.5 to about 2%.

The amount of active compound administered may be dependent on the subject being treated, the subject's weight, the manner of administration and the judgment of the prescribing physician. For example, a dosing schedule may involve the daily or semi-daily administration of the encapsulated compound at a perceived dosage of about 1 μg to about 1000 mg. In another embodiment, intermittent administration, such as on a monthly or yearly basis, of a dose of the encapsulated compound may be employed. Encapsulation facilitates access to the site of action and allows the administration of the active ingredients simultaneously, in theory producing a synergistic effect. In accordance with standard dosing regimens, physicians will readily determine optimum dosages and will be able to readily modify administration to achieve such dosages.

A therapeutically effective amount of a compound or composition disclosed herein can be measured by the therapeutic effectiveness of the compound. The dosages, however, may be varied depending upon the requirements of the patient, the severity of the condition being treated, and the compound being used. In one embodiment, the therapeutically effective amount of a disclosed compound is sufficient to establish a maximal plasma concentration. Preliminary doses as, for example, determined according to animal tests, and the scaling of dosages for human administration is performed according to art-accepted practices.

Toxicity and therapeutic efficacy can be determined by standard pharmaceutical procedures in cell cultures or experimental animals, for example, for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

Data obtained from the cell culture assays or animal studies can be used in formulating a range of dosage for use in humans. Therapeutically effective dosages achieved in one animal model may be converted for use in another animal, including humans, using conversion factors known in the art (see, for example, Freireich et al., Cancer Chemother. Reports 50(4):219-244 (1966) and Table 1 for Equivalent Surface Area Dosage Factors).

TABLE 1
Equivalent Surface Area Dosage Factors

To:

| | Mouse | Rat | Monkey | Dog | Human |
|---|---|---|---|---|---|
| From: | (20 g) | (150 g) | (3.5 kg) | (8 kg) | (60 kg) |
| Mouse | 1 | ½ | ¼ | ⅙ | 1/12 |
| Rat | 2 | 1 | ½ | ¼ | 1/7 |
| Monkey | 4 | 2 | 1 | ⅗ | ⅓ |
| Dog | 6 | 4 | ⅗ | 1 | ½ |
| Human | 12 | 7 | 3 | 2 | 1 |

The dosage of such compounds lies preferably within a range of circulating concentrations that include the ED₅₀ with little or no toxicity. The dosage may vary within this range depending upon the dosage form employed and the route of administration utilized. Generally, a therapeutically effective amount may vary with the subject's age, condition, and gender, as well as the severity of the medical condition in the subject. The dosage may be determined by a physician and adjusted, as necessary, to suit observed effects of the treatment.

In one embodiment, a compound of Formula I or II, or a tautomer, stereoisomer, pharmaceutically acceptable salt or hydrate thereof, is administered in combination with another therapeutic agent. The other therapeutic agent can provide additive or synergistic value relative to the administration of a compound of the invention alone. In certain embodiments, a compound of Formula I or II or a tautomer, stereoisomer, pharmaceutically acceptable salt or hydrate thereof, is administered in combination with one or more anti-cancer agents.

Therapeutic Methods

The invention provides methods of inhibiting BET proteins in a subject to treat or prevent a disease or disorder that is sensitive to a BET inhibitor. These methods comprise administering to a subject (for example, a mammal, such as a human) a therapeutically effective amount of at least one compound of Formula I or II, or a tautomer, stereoisomer, pharmaceutically acceptable salt or hydrate thereof. In another embodiment, at least one compound of Formula I or II, or tautomer, stereoisomer, pharmaceutically acceptable salt or hydrate thereof may be administered as a pharmaceutically acceptable composition, comprising one or more compounds of Formula I or II and a pharmaceutically acceptable carrier.

In some embodiments, the disease or disorder is a cancer which may be treated or prevented by administering a therapeutically effective amount of at least one compound of Formula I or II, or a tautomer, stereoisomer, pharmaceutically acceptable salt or hydrate thereof.

In certain embodiments, the cancer to be treated is a midline carcinoma. In some embodiments, the cancer is characterized by c-myc activation or overexpression. In other embodiments, the cancer is characterized by overexpression or activation of n-myc. In certain embodiments, the cancer is Burkitt's lymphoma, acute myelogenous leukemia, multiple myeloma, or aggressive human medulloblastoma. In some embodiments, the cancer relies on the recruitment of p-TEFb to regulate activated oncogenes such as, for example, NOTCH1. In some embodiments, the cancer to be treated or prevented by the methods of the invention is selected from the group consisting of hematological, epithelial including lung, breast and colon carcinomas, midline carcinomas, mesenchymal, hepatic, renal and neurological tumours.

The certain embodiments, administration of a compound of Formula I or Formula II or a tautomer, stereoisomer, pharmaceutically acceptable salt or hydrate thereof, to a mammal suffering from a cancer induces apoptosis in cancer cells by decreasing expression of the anti-apoptosis gene Bcl2. Thus, some embodiments of the invention provide a method of treating or preventing a disease or disorder in a mammal that benefits from increased cell death or differentiation, or decreased cell proliferation, comprising administering a compound of Formula I or Formula II or a tautomer, stereoisomer, pharmaceutically acceptable salt or hydrate thereof.

In some embodiments of the invention, the compound of Formula I or Formula II or a tautomer, stereoisomer, pharmaceutically acceptable salt or hydrate thereof is administered in combination with another anti-cancer agent, such as, for example, bortezomib, thalidomide, dexamethasone, 5-azacitidine, decitabine, vorinostat, or cyclophosphamide. In some embodiments, the anti-cancer agent is a PI3K or mTOR inhibitor, such as rapamycin or a rapamycin analog. In some embodiments, the anti-cancer agent is a gamma secretase inhibitor or an AMPK inducer, such as, for example, metformin or phenformin. In certain embodiments, the anti-cancer agent is an ornithine decarboxylase inhibitor, such as, for example, difluoromethylornithine.

At least one compounds of Formula I or Formula II or a tautomer, stereoisomer, pharmaceutically acceptable salt or hydrate thereof may also be administered to treat or prevent and immune or inflammatory diseases or disorders. In other embodiments, at least one compound of Formula I or Formula II, or a tautomer, stereoisomer, pharmaceutically acceptable salt or hydrate thereof may also be administered to treat or prevent a disease or disorder resulting from an infection by bacteria or virus, such as for example, HIV, HPV, or herpes. In some embodiments, the disease or disorder to be treated by the methods of the invention is AIDS. In other embodiments, the at least one compound of Formula I or Formula II, or a tautomer, stereoisomer, pharmaceutically acceptable salt or hydrate thereof is administered to treat or prevent sepsis in a mammal.

### EXAMPLES

The invention is further illustrated by the following non-limiting examples, wherein the following abbreviations have the following meanings. If an abbreviation is not defined, it has its generally accepted meaning.

AcOH=acetic acid

BINAP=2,2′-bis(diphenylphosphino)-1,1′-binaphthyl

Boc=N-tert-butoxycarbonyl

TBDMS=tert-butyldimethylsilyl

dba=dibenzylidene acetone

DCM=dichloromethane

DMAP=dimethylaminopyridine

DMF=dimethylformamide

DMSO=dimethylsulfoxide

EDCI=1-ethyl-3-(3′-dimethylaminopropyl)carbodiimide

EtOH=ethanol

EtOAc=ethyl acetate

IBX=2-Iodoxybenzoic acid

MeOH=methanol

HOBt=N-hydroxybenzotriazole

THF=tetrahydrofuran

TEA=triethylamine

p-TSA=p-toluenesulfonic acid

TBAF=tetrabutylammonium fluoride

DMA=N,N-dimethylacetamide

DIBAL-H=diisobutylaluminum hydride

TPAP=tetrapropylammonium perruthenate

NMO=N-methylmorpholine N-oxide

DDQ=2,3-dicyano-5,6-dichloro-parabenzoquinone

DME=1,2-dimethoxyethane

TFA=trifluoroacetic acid

DPPF=1,1′-bis(diphenylphosphino)ferrocene

Pd(OAc)₂=palladium(II) acetate

Pd(PPh₃)₄=tetrakis(triphenylphosphine)palladium(0)

### Example 1

### Preparation of 5-(2-dimethylamino-ethoxy)-2(4-hydroxy-3,5-dimethylphenyl)-7-methoxy-3H-quinazolin-4-one

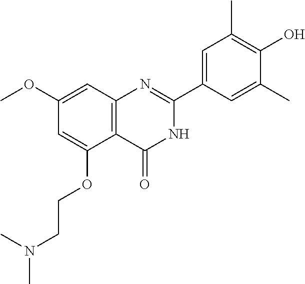

To a solution of 3,5-dimethyl-4-hydroxybenzaldehyde (10.0 g, 66.6 mmol) in anhydrous DMF (20 mL) was added NaH (4.00 g, 99.9 mmol) in portions and the mixture was stirred for 1 hour at room temperature. Benzyl bromide (9.5 mL, 80 mmol) was added dropwise and stirred for 16 hours at room temperature. Water was added, the mixture was acidified with acetic acid to pH approximately 4-5, and the product was isolated by extraction with ethyl acetate. The solvent was evaporated in vacuo and the residue was purified by column chromatography (silica gel 230-400 mesh; 2-5% ethyl acetate/hexane as eluent) to give 4-benzyloxy-3,5-dimethyl-benzaldehyde as white solid. Yield: 15.2 g (95%).

A mixture of 2-amino-4,6-difluorobenzamide (2.13 g, 12.4 mmol), 4-benzyloxy-3,5-dimethylbenzaldehyde (2.98 g, 12.4 mmol), NaHSO₃ (2.50 g, 13.6 mmol) and p-toluene sulfonic acid (0.236 g, 1.24 mmol) in N,N-dimethylacetamide (20 mL) was stirred at 110-120° C. for 16 hours. The solvent was evaporated in vacuo, water was added and the precipitated solid was filtered off, washed with water and ether to give 2-(4-benzyloxy-3,5-dimethylphenyl)-5,7-difluoro-3H-quinazolin-4-one as a light yellow solid. Yield: 1.99 g (41%).

To a solution of 2-dimethylaminoethanol (180 mg, 2.03 mmol) in DMF (2 mL) was added NaH (61 mg, 1.5 mmol) at 0° C. The reaction mixture was stirred at room temperature for 30 minutes. Then, 2-(4-benzyloxy-3,5-dimethylphenyl)-5,7-difluoro-3H-quinazolin-4-one (200 mg, 0.510 mmol) was added and the reaction mixture was stirred at room temperature for 16 hours. The reaction mixture was diluted with water and extracted with ethyl acetate. The combined organic layers were washed with water, brine, dried over Na₂SO₄, and concentrated in vacuo to give product 2-(4-bezyloxy-3,5-dimethyl phenyl)-5-(2-dimethylamino-ethoxy)-7-fluoro-3H-quinazolin-4-one. Yield: 220 mg (93%).

To a solution of 2-(4-bezyloxy-3,5-dimethylphenyl)-5-(2-dimethylaminoethoxy)-7-fluoro-3H-quinazolin-4-one (220 mg, 0.470 mmol) in DMF (3 mL) was added 25% (w/w) sodium methoxide in methanol (205 mg, 3.81 mmol). The reaction mixture was heated at 95° C. for 4 hours. The reaction mixture was cooled to the room temperature, diluted with water, and extracted with ethyl acetate. The combined organic layer was washed with water, brine, dried over anhydrous Na₂SO₄, and concentrated in vacuo to give crude product, which was purified by column chromatography (silica gel 230-400 mesh; 5% NH₃ in methanol/CH₂Cl₂ as eluent) to give pure product 2-(4-bezyloxy-3,5-dimethylphenyl)-5-(2-dimethylamino-ethoxy)-7-methoxy-3H-quinazolin-4-one. Yield: 110 mg (49%).

To a solution of 2-(4-bezyloxy-3,5-dimethylphenyl)-5-(2-dimethylaminoethoxy)-7-methoxy-3H-quinazolin-4-one (110 mg, 0.23 mmol) in methanol (5 mL) and THF (5 mL) was added Pd/C (50 mg, 10% on charcoal). The reaction mixture was hydrogenated for 2 hours at 50 psi at room temperature. The mixture was filtered through celite and solvent was evaporated in vacuo to give crude product, which was purified by column chromatography (silica gel 230-400 mesh; 5% NH₃ in methanol/CH₂Cl₂ as eluent) to give the title compound as a light brown solid. Yield: 70 mg (78%). ¹H NMR (400 MHz, CDCl₃): δ 7.58 (s, 2H), 6.80 (s, 1H), 6.40 (s, 1H), 4.20 (t, 2H), 3.90 (s, 3H), 2.90 (t, 2H), 2.40 (s, 3H), 2.25 (s, 3H). MS (ES⁺) m/z: 384.09 (M+1).

### Example 2

### Preparation of 2-(4-hydroxy-3,5-dimethyl-phenyl)-7-methoxy-5-(2-methoxy-ethoxy)-3H-quinazolin-4-one

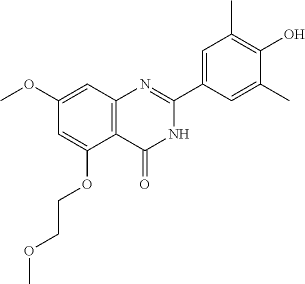

To a solution of 2-methoxy-ethanol (2 mL) in anhydrous DMF (2 mL) was added NaH (0.276 g, 6.90 mmol) in portions at 0° C. The reaction mixture was allowed to warm to room temperature and stirred for 30 minutes. The compound 2-(4-benzyloxy-3,5-dimethyl-phenyl)-5,7-difluoro-3H-quinazolin-4-one (0.25 g, 0.64 mmol) was added and the reaction mixture was stirred at room temperature for 16 hours. Water was added and the mixture was acidified with acetic acid to pH approximately 4-5. The precipitated solid was filtered off and washed with water and dried over anhydrous Na₂SO₄ to give 2-(4-benzyloxy-3,5-dimethyl-phenyl)-7-fluoro-5-(2-methoxy-ethoxy)-3H-quinazolin-4-one as a white solid. Yield: 0.28 g (98%).

To a solution of 2-(4-benzyloxy-3,5-dimethyl-phenyl)-7-fluoro-5-(2-methoxy-ethoxy)-3H-quinazolin-4-one (0.28 g, 0.62 mmol) in anhydrous DMF (3 mL) was added a 25% solution of sodium methoxide in methanol (1.5 mL, 7.0 mmol) and the reaction mixture was heated to 80-90° C. for 6 hours. Water was added and the mixture was acidified with acetic acid, to pH approximately 4-5. The precipitated solid was filtered off and purified by column chromatography (silica gel 230-400 mesh; 20-50% ethyl acetate/CH₂Cl₂ as eluent) to give 2-(4-benzyloxy-3,5-dimethyl-phenyl)-7-methoxy-5-(2-methoxy-ethoxy)-3H-quinazolin-4-one as a white solid. Yield: 0.1 g (35%).

The compound 2-(4-benzyloxy-3,5-dimethyl-phenyl)-7-methoxy-5-(2-methoxy-ethoxy)-3H-quinazolin-4-one (0.1 g, 0.22 mmol) was hydrogenated in THF/methanol (20/20 mL) at room temperature using Pd/C (10 wt %, 0.05 g) for 4 hours. After filtering through celite, the solvent was evaporated in vacuo and the crude material was purified by column chromatography (silica gel 230-400 mesh; 20-50% ethyl acetate/CH₂Cl₂ as eluent) to give the title compound as a white solid. Yield: 0.05 g (61.7%). ¹H NMR (400 MHz, DMSO-d₆): δ 7.81 (s, 2H), 6.70 (s, 1H), 6.51 (s, 1H), 4.19 (t, 2H), 3.87 (s, 3H), 3.70 (t, 2H), 3.40 (s, 3H), 2.21 (s, 6H). MS (ES⁺) m/z: 371.11 (M+1).

### Example 3

### Preparation of 7-(2-amino-ethoxy)-2-(4-hydroxy-3,5-dimethyl-phenyl)-5-methoxy-3H-quinazolin-4-one

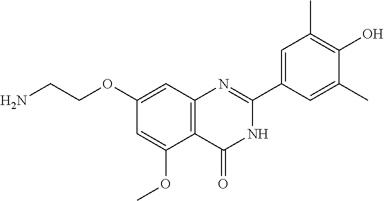 [SMILES: COc1cc(OCCN)cc2nc(-c3cc(C)c(O)c(C)c3)[nH]c(=O)c12]

To a solution of 2-amino-4,6-difluoro-benzamide (0.400 g, 2.32 mmol) and 4-benzyloxy-3,5-dimethylbenzaldehyde (0.560 g, 2.32 mmol) in N,N-dimethylacetamide (5 mL) were added NaHSO₃ (0.450 g, 2.55 mmol) and p-TSA (44 mg, 0.23 mmol) and the reaction mixture was heated at 115-120° C. for 16 hours. The reaction mixture was cooled to room temperature. N,N-Dimethylacetamide was removed under reduced pressure. The residue was diluted with water and the solid was collected and mixed and stirred for 0.5 hours with methanol (20 mL). The solid was filtered to give 2-(4-benzyloxy-3,5-dimethyl-phenyl)-5,7-difluoro-3H-quinazolin-4-one. Yield: 0.41 g (45%).

A solution of 2-(4-benzyloxy-3,5-dimethyl-phenyl)-5,7-difluoro-3H-quinazolin-4-one (0.39 g, 1.0 mmol) and 25% sodium methoxide in methanol (0.70 g, 3.2 mmol) in DMF (1.5 mL) was stirred at room temperature for 16 hours. Acetic acid (1.0 mL) was added and the mixture was poured into water (20 mL) and stirred for 0.5 hours. The solid was filtered and further rinsed with water (30 mL), and dried to give 2-(4-benzyloxy-3,5-dimethyl-phenyl)-7-fluoro-5-methoxy-3H-quinazolin-4-one. Yield: 0.39 g (92%).

To a solution of 2-(4-benzyloxy-3,5-dimethyl-phenyl)-7-fluoro-5-methoxy-3H-quinazolin-4-one (0.390 g, 0.960 mmol) and 2-dimethylamin-ethanol (0.258 g, 2.89 mmol) in DMF (1.5 mL) was added sodium hydride (0.135 g, 2.97 mmol). The reaction mixture was kept at 80° C. for 16 hours and then poured into water (20 mL). The aqueous layer was adjusted to pH 9.0, and extracted with dichloromethane. The crude product was purified by column chromatography on silica gel (230-400 mesh) using 10% methanol in dichloromethane with 1% triethylamine as eluent to give 7-(2-amino-ethoxy)-2-(4-benzyloxy-3,5-dimethyl-phenyl)-5-methoxy-3H-quinazolin-4-one. Yield: 0.25 g (58%).

To a solution of 7-(2-amino-ethoxy)-2-(4-benzyloxy-3,5-dimethyl-phenyl)-5-methoxy-3H-quinazolin-4-one (0.25 g, 0.56 mmol) in methanol (15 mL) was added 10% palladium charcoal wet (0.17 g) and the reaction mixture was subjected to hydrogenation under hydrogen balloon at room temperature for 16 hours. The catalyst was filtered through celite and methanol was removed. The resulting material was further washed with an ethyl acetate and ether mixture (20 mL/20 mL) to give the title compound. Yield: 0.13 g (75%). ¹H NMR (400 Hz, DMSO-d₆): δ 11.70 (s, 1H), 8.98 (s, 1H), 7.83 (s, 2H), 6.78 (s, 1H), 6.48 (s, 1H), 4.25 (t, 2H), 3.82 (s, 3H), 2.81 (t, 2H), 2.35 (s, 6H), 2.24 (s, 6H). MS (ES⁺) m/z: 384.14 (M+1).

### Example 4

### Preparation of 2-(4-hydroxy-3,5-dimethyl-phenyl)-5-methoxy-7-(2-methoxy-ethoxy)-3H-quinazolin-4-one

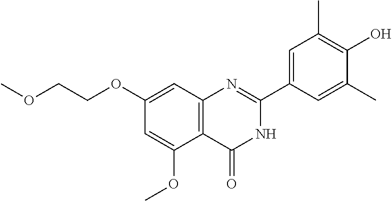

Sodium hydride (0.340 g, 8.62 mmol) was taken in anhydrous DMF (5 mL). Anhydrous 2-methoxy-ethanol (1.64 g, 21.6 mmol) was added dropwise at 0° C. under nitrogen over a period of 15 minutes. Stirring was continued at 0° C. for 5 minutes. The ice-bath was removed and stirring continued at room temperature for 10 minutes. Then, 2-(4-benzyloxy-3,5-dimethyl-phenyl)-7-fluoro-5-methoxy-3H-quinazolin-4-one (0.436 g, 1.08 mmol) was added. The color changed to green and stirring continued at 100° C. for 4 hours (progress of the reaction was monitored by TLC). The reaction mixture was cooled to room temperature, then quenched with glacial acetic acid (2 mL). Water (75 mL) was added. A white precipitate formed, which was filtered, washed with water, and dried under vacuum. Crude compound was purified by column chromatography (silica gel 230-400 mesh; 0-3% methanol in CH₂Cl₂ as eluent) to give 2-(4-benzyloxy-3,5-dimethyl-phenyl)-5-methoxy-7-(2-methoxy-ethoxy)-3H-quinazolin-4-one as a white solid. Yield: 0.09 g (18%).

To a solution of 2-(4-benzyloxy-3,5-dimethyl-phenyl)-5-methoxy-7-(2-methoxy-ethoxy)-3H-quinazolin-4-one (0.083 g, 0.18 mmol) in methanol (15 mL) and THF (5 mL) was added palladium on charcoal (75 mg). The reaction mixture was hydrogenated at 50 psi for 16 hours at room temperature then filtered through celite. The filtrate was concentrated under reduced pressure and the crude compound was purified by preparative HPLC to give the title compound as a white solid. Yield: 0.043 g (45%). ¹H NMR (400 MHz, CDCl₃): δ 7.80 (s, 2H), 7.00 (s, 1H), 6.52 (s, 1H), 4.20 (m, 2H), 3.80 (m, 5H), 3.48 (s, 3H), 2.22 (s, 6H).

### Example 5

### Preparation of 7-(2-benzyloxy-ethoxy)-2-(4-hydroxy-3,5-dimethyl-phenyl)-5-methoxy-3H-quinazolin-4-one

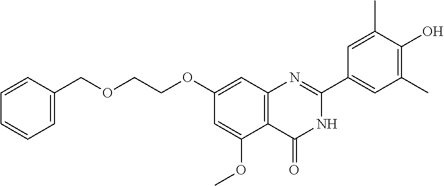 [SMILES: COc1cc(OCCOCc2ccccc2)cc2nc(-c3cc(C)c(O)c(C)c3)[nH]c(=O)c12]

To a suspension of sodium hydride (2.00 g, 50.0 mmol) in anhydrous DMF (30 mL) at 0° C. was added a solution of 4-hydroxy-3,5-dimethyl-benzaldehyde (5.00 g, 33.3 mmol) in anhydrous DMF (20 mL), dropwise over a period of 30 minutes, under nitrogen. Stirring continued at room temperature for 30 minutes and the mixture was cooled to 0° C. Chloromethoxymethane (5.06 mL, 66.6 mmol) was added and the reaction mixture was stirred at room temperature for 16 hours under nitrogen. The reaction mixture was poured into water (200 mL), extracted with ethyl acetate (2×50 mL), dried over anhydrous Na₂SO₄, and concentrated. The crude compound was purified by column chromatography (SiO₂, ethyl acetate/hexanes=1:3) to afford 4-methoxymethoxy-3,5-dimethyl-benzaldehyde as colorless oil. Yield: 5.97 g (92%).

To a solution of 4-methoxymethoxy-3,5-dimethyl-benzaldehyde (4.00 g, 20.6 mmol) and 2-amino-4,6-difluoro-benzamide (3.55 g, 20.6 mmol) in N,N-dimethylacetamide (20 mL) were added sodium hydrogen sulfite (58.5 wt %) (5.45 g, 30.9 mmol) and p-toluenesulfonic acid (0.20 g, 1.0 mmol). The reaction mixture was stirred at 120° C. for 16 hours under nitrogen and cooled to room temperature. The solvent was evaporated under reduced pressure. Methanol (50 mL) and water (200 mL) were added, the separated solid was filtered, washed with water (30 mL), methanol (30 mL), hexanes (100 mL), and dried under vacuum, to afford 5,7-difluoro-2-(4-methoxymethoxy-3,5-dimethyl-phenyl)-3H-quinazolin-4-one as a white solid. Yield: 1.40 g (20%).

To a solution of 5,7-difluoro-2-(4-methoxymethoxy-3,5-dimethyl-phenyl)-3H-quinazolin-4-one (1.40 g, 4.04 mmol) in anhydrous DMF (20 mL) was added a solution of sodium methoxide in methanol (25 wt %, 5.0 mL, 24 mmol). The reaction mixture was stirred at room temperature for 16 hours under nitrogen, diluted with water (100 mL), extracted with ethyl acetate, dried over sodium sulfate, and concentrated on a rotary evaporator to afford 7-fluoro-5-methoxy-2-(4-methoxymethoxy-3,5-dimethyl-phenyl)-3H-quinazolin-4-one as a white solid. Yield: 1.1 g (76%).

To a suspension of sodium hydride (0.176 g, 4.40 mmol) in anhydrous DMF (20 mL) was added benzyloxyethanol (1.02 g, 6.70 mmol) at room temperature under nitrogen. The reaction mixture was stirred 60° C. for 30 minutes to get a clear solution. Then, 7-fluoro-5-methoxy-2-(4-methoxymethoxy-3,5-dimethyl-phenyl)-3H-quinazolin-4-one (0.200 g, 0.559 mmol) was added and the reaction mixture was stirred at 105° C. for 16 hours under nitrogen. The reaction was diluted with water (100 mL), extracted with ethyl acetate (100 mL), and concentrated on a rotary evaporator. The oily residue was subjected to column chromatography (SiO₂, hexanes/ethyl acetate/methanol=6:2:1) to afford a mixture of two components of very similar polarity. The mixture was dissolved in 50% aqueous acetic acid (60 mL) and mixed with concentrated HCl (3 mL). The resulting mixture was stirred at 70° C. for 1 hour and concentrated to dryness on a rotary evaporator. The residue was diluted with saturated sodium bicarbonate aqueous solution (50 mL), extracted with ethyl acetate (150 mL), and concentrated. The residue was purified by column chromatography (SiO₂, hexanes/ethyl acetate/methanol=7:2:1) to afford the title compound as a light yellow solid. Yield: 45 mg (18%). ¹H NMR (400 MHz, CDCl₃): δ 9.68 (br s, 1H), 7.69 (s, 2H), 7.40-7.30 (m, 5H), 6.79 (d, 1H), 6.50 (d, 1H), 4.66 (s, 2H), 4.27 (t, 2H), 3.96 (s, 3H), 3.88 (t, 2H), 2.33 (s, 6H). MS (ES⁺) m/z: 447.59 (M+1).

### Example 6

### Preparation of 2-(4-hydroxy-3,5-dimethylphenyl)-5-methoxy-7-[2-(pyridin-3-ylmethoxy)ethoxy]-3H-quinazolin-4-one

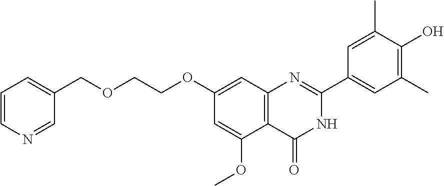 [SMILES: COc1cc(OCCOCc2cccnc2)cc2nc(-c3cc(C)c(O)c(C)c3)[nH]c(=O)c12]

To a stirred solution of 5,7-difluoro-2-(4-methoxymethoxy-3,5-dimethylphenyl)-3H-quinazolin-4-one (1.04 g, 3.00 mmol) in anhydrous DMF (10 mL) was added a solution of sodium methoxide (25 wt %) in methanol (3.9 mL, 18.0 mmol) at room temperature. The reaction mixture was stirred at room temperature for 16 hours under nitrogen. Water (100 mL) was added, the white precipitated solid was filtered off, washed with water and dried under vacuum. The solid was further washed with 10% methanol in ether (20 mL), then ether (20 mL), and dried under vacuum. Yield 0.95 g (88%).

Sodium hydride (60% in mineral oil; 1.00 g, 25.0 mmol) was added slowly to ethylene glycol (1.48 g, 239 mmol), cooled to 0° C. under nitrogen. The cooling bath was removed, and the mixture was stirred for a further 15 minutes at room temperature, before 3-(bromomethyl)pyridine hydrobromide (2.53 g, 10.0 mmol) was added. Then, the mixture was stirred at room temperature for 2.5 days. Water was added, the mixture was extracted with EtOAc (5×100 mL), the extracts were washed with brine, dried over anhydrous Na₂SO₄, and concentrated under vacuum. Purification by column chromatography on silica gel, with CH₂Cl₂/MeOH (95:5) as the eluent, gave 2-(pyridin-3-ylmethoxy)-ethanol as a colorless liquid. Yield 0.90 g, 59%.

To a solution of 7-fluoro-5-methoxy-2-(4-methoxymethoxy-3,5-dimethyl-phenyl)-3H-quinazolin-4-one (0.30 g, 0.86 mmol) and 2-(pyridin-3-ylmethoxy)ethanol (0.20 g, 1.3 mmol) in DMF (2.0 mL), was added sodium hydride (60% in mineral oil) (0.30 g, 6.9 mmol). The mixture was stirred at room temperature under nitrogen for 3 h, then in an oil bath at 95° C. for 2.5 days. The mixture was concentrated under vacuum, water (approximately 50 mL) was added, and the mixture extracted with dichloromethane (3×50 mL). The dichloromethane solution was dried over anhydrous Na₂SO₄, concentrated under vacuum, and purified by column chromatography on silica gel, with CH₂Cl₂/MeOH (95:5) as eluent, to give 5-methoxy-2-(4-methoxymethoxy-3,5-dimethylphenyl)-7-[2-(pyridin-3-ylmethoxy)-ethoxy]-3H-quinazolin-4-one. Yield 150 mg (35%).

To a solution of 5-methoxy-2-(4-methoxymethoxy-3,5-dimethylphenyl)-7-[2-(pyridin-3-ylmethoxy)ethoxy]-3H-quinazolin-4-one (0.10 g, 0.20 mmol) in acetic acid (10 mL) and water (10 mL), sulphuric acid (0.5 mL) was added. The solution was stirred in a 75° C. oil bath for 5 hours. The mixture was then concentrated under reduced pressure. The residue was dissolved in methanol, and 2 M Na₂CO₃ was added until the pH reached 8. The mixture was concentrated under reduced pressure. The resulting precipitate was filtered, washed with water, and dried in air. The precipitate was washed further with methanol to give the title compound. Yield: 67 mg (74%). ¹H NMR (400 MHz, DMSO-d₆): δ 11.69 (s, 1H), 8.95 (s, 1H), 8.59 (s, 1H), 8.51 (d, J=3.2 Hz, 1H), 7.84 (s, 2H), 7.79 (dt, J=7.6 and 2.0 Hz, 1H), 7.41-7.38 (m, 1H), 6.72 (d, J=2.0 Hz, 1H), 6.49 (d, J=2.4 Hz, 1H), 4.63 (s, 2H), 4.30 (m, 2H), 3.86 (m, 2H), 3.83 (s, 3H), 2.23 (s, 6H). MS (ES⁻) m/z: 446.52 (M−1).

### Example 7

### Preparation of 7-(2-dimethylamino-ethoxy)-2-(4-hydroxy-3,5-dimethylphenyl)-3H-quinazolin-4-one

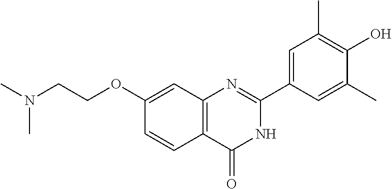

To a solution of 2-amino-4-fluoro-benzamide (0.77 g, 5.00 mmol) and 4-benzyloxy-3,5-dimethyl-benzaldehyde (1.20 g, 5.00 mmol) in N,N-dimethyl acetamide (20 mL) were added sodium hydrogen sulfite (58.5 wt %, 1.10 g, 6.00 mmol) and p-toluenesulfonic acid monohydrate (0.19 g, 1.00 mmol). The reaction mixture was stirred at 120° C. for 16 hours under nitrogen, and then cooled to room temperature. Solvent was evaporated under reduced pressure, and water (100 mL) was added. The separated solid was filtered, washed with water (50 mL), and dried under vacuum to give a white solid. Yield: 0.74 g (39%).

Sodium hydride (60% suspension in mineral oil; 0.36 g, 9.00 mmol) was taken in anhydrous DMF (20 mL). Then, 2-dimethylamino-ethanol (1.07 g, 12.0 mmol) was added drop-wise at room temperature under nitrogen. After the addition, the reaction mixture was stirred at room temperature for 20 minutes. Then, 2-(4-benzyloxy-3,5-dimethylphenyl)-7-fluoro-3H-quinazolin-4-one (0.56 g, 1.50 mmol) was added and the reaction mixture was stirred at 80° C. for 16 hours. The reaction mixture was cooled to room temperature. Water (100 mL) was added and the mixture was neutralized to pH approximately 8 with aqueous 2 N HCl. The separated solid was filtered, washed with water, and dried under vacuum. The crude compound was purified by the Simpliflash system (0-5% methanol in CH₂Cl₂ and 7 N ammonia in methanol 5% in CH₂Cl₂ as eluent) to give 2-(4-benzyloxy-3,5-dimethylphenyl)-7-(2-dimethylamino-ethoxy)-3H-quinazolin-4-one as a white solid. Yield: 0.32 g (48%).

2-(4-Benzyloxy-3,5-dimethylphenyl)-7-(2-dimethylamino-ethoxy)-3H-quinazolin-4-one (0.30 g, 11.2 mmol) was dissolved in a mixture of methanol and THF (1:1, 60 mL). Palladium on carbon (10 wt %, 0.20 g) was added and the reaction mixture was hydrogenated at 45 psi for 6 hours. The reaction mixture was filtered, and the filtrate was concentrated. The residue was washed with 10% methanol in ether, then ether, and dried under vacuum to give the title compound as a white solid. Yield: 0.18 g (75%). ¹H NMR (400 MHz, DMSO-d₆): δ 11.98 (br s, 1H), 8.94 (br s, 1H), 7.99 (d, J=8.59 Hz, 1H), 7.86 (s, 2H), 7.13 (s, 1H), 7.01 (d, J=8.98 Hz, 1H), 4.21 (t, J=5.46 Hz, 2H), 2.68 (t, J=5.27 Hz, 2H), 2.24 (s, 12H). MS (ES+) m/z 354.16 (100%).

### Example 8

### Preparation of 2-(4-hydroxy-3,5-dimethyl-phenyl)-6-(pyridin-4-ylamino)-3H-quinazolin-4-one

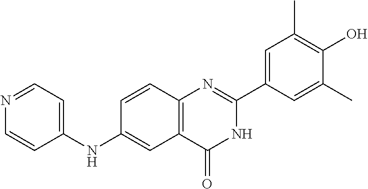 [SMILES: Cc1cc(-c2nc3ccc(Nc4ccncc4)cc3c(=O)[nH]2)cc(C)c1O]

To a solution of 6-amino-2-(4-hydroxy-3,5-dimethyl-phenyl)-3H-quinazolin-4-one (300 mg, 1.07 mmol) in pyridine (3 mL), were added 4-bromopridinium hydrochloride (207 mg, 1.07 mmol), Pd₂(dba)₃ (19 mg, 0.02 mmol), dppf (18 mg, 0.03 mmol) and NaO-t-Bu (328 mg, 3.41 mmol). The reaction mixture was heated at 140° C. for 1 hour in a microwave oven. Solvent was removed under reduced pressure. The crude compound was purified by the Simpliflash system (5% 7 N ammonia in methanol and dichloromethane as eluent) to give the title compound as a yellow solid. Yield: 58 mg (15%). ¹H NMR (400 MHz, DMSO-d₆): δ 12.13 (s, 1H), 9.16 (s, 1H), 8.92 (s, 1H), 8.25 (br s, 2H), 7.84 (d, J=2.0 Hz, 1H), 7.81 (s, 2H), 7.65 (m, 2H), 6.99 (d, J=5.2 Hz, 2H), 2.22 (s, 6H). MS (ES) m/z: 359.26 (M+1) (100%).

### Example 9

### Preparation of 2-(4-hydroxy-3,5-dimethyl-phenyl)-6-(pyridin-2-ylamino)-3H-quinazolin-4-one

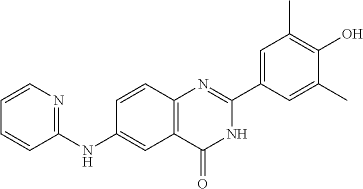 [SMILES: Cc1cc(-c2nc3ccc(Nc4ccccn4)cc3c(=O)[nH]2)cc(C)c1O]

To a solution of 6-amino-2-(4-hydroxy-3,5-dimethyl-phenyl)-3H-quinazolin-4-one (300 mg, 1.07 mmol) in pyridine (3.5 mL), were added 2-bromopyridine (202 mg, 1.28 mmol), Pd₂(dba)₃ (20 mg, 0.02 mmol), dppf (18 mg, 0.03 mmol) and NaO-t-Bu (329 mg, 3.42 mmol). The reaction mixture was heated at 125° C. for 1 hour in a microwave oven (100 W). Solvent was removed under reduced pressure. The crude compound was purified by column chromatography (silica gel 230-400 mesh; 3% methanol, 37% ethyl acetate and 60% CH₂Cl₂ as eluent). The compound was further purified by preparative HPLC to give the title compound as a beige-colored solid. Yield: 35 mg (9%). ¹H NMR (400 MHz, DMSO-d₆): δ 12.01 (br s, 1H), 9.40 (s, 1H), 8.87 (br s, 1H), 8.60 (d, J=2.34 Hz, 1H), 8.23 (d, J=3.91 Hz, 1H), 7.97 (dd, J=8.99 and 2.74 Hz, 1H), 7.82 (s, 2H), 7.72-7.44 (m, 2H), 6.87 (d, J=8.60 Hz, 1H), 6.83-6.78 (m, 1H), 2.23 (s, 6H). MS (ES) m/z: 359.01 (M+1) (100%).

### Example 10

### Preparation of 2-(4-hydroxy-3,5-dimethylphenyl)-6-((4-methylpiperazin-1-yl)methyl)quinazolin-4(3H)-one

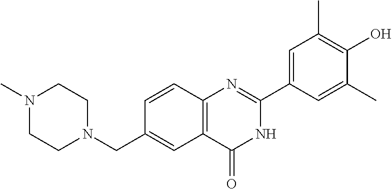

A solution of 2-amino-5-bromobenzamide (12.0 g, 55.8 mmol) and 4-hydroxy-3,5-dimethylbenzaldehyde (8.4 g, 55.8 mmol) in DMA (200 mL) was treated with NaHSO₃ (7.7 g, 72.5 mmol) and p-TsOH (1.1 g, 5.6 mmol). The reaction was heated at 135° C. for 2.5 hours, at which time, H₂O (10 mL) and CH₂Cl₂ (100 mL) were added and the solids were collected by filtration. The solids were washed with CH₂Cl₂ and dried in vacuo to afford 6-bromo-2-(4-hydroxy-3,5-dimethylphenyl)quinazolin-4(3H)-one (13.1 g, 68%).

A solution of 6-bromo-2-(4-hydroxy-3,5-dimethylphenyl)quinazolin-4(3H)-one (2.0 g, 5.8 mmol) in DMF (20 mL) was treated with vinyltributyltin (2.6 mL, 8.70 mmol), Pd(PPh₃)₄ (0.670 g, 0.58 mmol), and LiCl (0.730 g, 17.4 mmol). The reaction was stirred at reflux for 30 minutes, then concentrated in vacuo. The residue was purified by flash chromatography on silica gel, eluting with 30% to 100% of 92:7:1 CHCl₃/MeOH/concentrated NH₄OH in CH₂Cl₂, to afford 2-(4-hydroxy-3,5-dimethylphenyl)-6-vinylquinazolin-4(3H)-one (0.780 g, 46%).

To a suspension of 2-(4-hydroxy-3,5-dimethylphenyl)-6-vinylquinazolin-4(3H)-one (0.500 g, 1.70 mmol) in THF (30 mL) and H₂O (10 mL) was added NaIO₄ (1.09 g, 5.10 mmol), followed by OsO₄ (0.2 mL, 0.017 mmol). The reaction was stirred overnight, then concentrated in vacuo. The residue was purified by flash chromatography on silica gel, eluting with 92:7:1 to 6:3:1 CHCl₃/MeOH/concentrated NH₄OH to afford 2-(4-hydroxy-3,5-dimethylphenyl)-4-oxo-3,4-dihydroquinazoline-6-carbaldehyde (0.475 g, 95%).

To a solution of 2-(4-hydroxy-3,5-dimethylphenyl)-4-oxo-3,4-dihydroquinazoline-6-carbaldehyde (0.115 g, 0.40 mmol) in DCE/CH₂Cl₂ (1:1, 15 mL) was added 1-methylpiperazine (0.13 mL, 1.20 mmol) and NaBH(OAc)₃ (0.250 g, 1.20 mmol). The reaction stirred at room temperature overnight. After this time, the mixture was concentrated in vacuo and purified by flash chromatography on silica gel eluting with 92:7:1 CHCl₃/MeOH/concentrated NH₄OH to afford the title compound (0.036 g, 25%) as a white solid: ¹H NMR (300 MHz, DMSO-d₆): δ 11.63 (br s, 1H), 8.77 (br s, 1H), 8.00 (s, 1H), 7.85 (s, 2H), 7.65-7.69 (m, 2H), 3.57 (s, 2H), 2.15-2.39 (m, 17H); APCI MS m/z 377 [M−H]⁻.

### Example 11

### Preparation of N-((2-(4-hydroxy-3,5-dimethylphenyl)-4-oxo-3,4-dihydroquinazolin-6-yl)methyl)methanesulfonamide

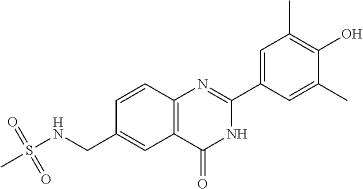

To a solution of methyl-5-methyl-2-nitrobenzoate (2.3 g, 11.8 mmol) in CHCl₃ (150 mL) was added NBS (5.3 g, 30.0 mmol) and benzoyl peroxide (0.285 g, 1.2 mmol). The reaction was heated at reflux temperature overnight. Then, the resulting mixture was washed sequentially with H₂O, Na₂CO₃, and brine. The organic layer was then dried (Na₂SO₄), filtered, and concentrated in vacuo. Purification by flash chromatography on silica gel, eluting with 5% to 20% ethyl acetate/heptane, afforded methyl 5-(bromomethyl)-2-nitrobenzoate (1.3 g, 40%).

To a solution of methyl 5-(bromomethyl)-2-nitrobenzoate (1.3 g, 4.7 mmol) in DMF (15 mL) was added potassium phthalimide (1.0 g, 5.2 mmol) and the reaction was stirred at room temperature for 1 hour and concentrated in vacuo. Purification by flash chromatography, eluting with 15% to 70% ethyl acetate/heptane, afforded methyl 5-((1,3-dioxoisoindolin-2-yl)methyl)-2-nitrobenzoate (1.4 g, 88%).

A solution of methyl 5-((1,3-dioxoisoindolin-2-yl)methyl)-2-nitrobenzoate (0.50 g, 1.4 mmol) in EtOH (10 mL) was treated with hydrazine (0.14 mL, 4.4 mol) and the reaction was stirred at room temperature overnight. After this time, the mixture was concentrated in vacuo and purified by flash chromatography on silica gel, eluting with 30% to 100% of 92:7:1 CHCl₃/MeOH/concentrate NH₄OH in CH₂Cl₂, to afford methyl 5-(aminomethyl)-2-nitrobenzoate (0.23 g, 78%).

To a solution of methyl 5-(aminomethyl)-2-nitrobenzoate (0.23 g, 1.1 mmol) in CH₂Cl₂ (5 mL) was added Et₃N (0.31 mL, 2.2 mmol) and methanesulfonyl chloride (0.08 mL, 1.1 mmol). The reaction was stirred for 15 minutes at room temperature, concentrated in vacuo, and purified by flash chromatography on silica gel, eluting with 2% to 20% MeOH/CH₂Cl₂, to afford methyl 5-(methylsulfonamidomethyl)-2-nitrobenzoate (0.18 g, 57%).

A mixture of methyl 5-(methylsulfonamidomethyl)-2-nitrobenzoate (0.18 g, 0.62 mmol) in EtOH (10 mL) was flushed with N₂. Pd/C (0.018 g) was added and the reaction was flushed with H₂ for 2 hours. Then, the resulting mixture was filtered through celite and the filtrate was concentrated. Purification by flash chromatography, eluting with 15% to 60% of 92:7:1 CHCl₃/MeOH/concentrate NH₄OH in CH₂Cl₂, afforded methyl 2-amino-5-(methylsulfonamidomethyl)-benzoate (0.085 g, 53%).

To a solution of methyl 2-amino-5-(methylsulfonamidomethyl)benzoate (0.085 g, 0.33 mmol) in THF (7 mL) and H₂O (3 mL) was added LiOH.H₂O (0.028 g, 0.65 mol). The reaction was stirred at room temperature for 2 hours and then neutralized with 1 N HCl. The resulting aqueous solution was extracted with EtOAc. The organics were washed with brine, dried (Na₂SO₄), filtered, and concentrated, to afford 2-amino-5-(methylsulfonamidomethyl)benzoic acid (0.066 g, 82%).

A solution of 2-amino-5-(methylsulfonamidomethyl)benzoic acid (0.066 g, 0.27 mol) in THF (5 mL) was treated with EDCI (0.062 g, 0.32 mmol), HOBT (0.044 g, 0.32 mol) and NMM (0.035 mL, 0.32 mmol.) The reaction was stirred at room temperature for 1.5 hours. Then, NH₄OH (0.03 mL, 0.35 mmol) in H₂O (0.03 mL) was added. The mixture was stirred at room temperature for 5 hours and then concentrated. Purification by flash chromatography, eluting with 92:7:1 to 7:2.5:0.5 CHCl₃/MeOH/concentrated NH₄OH, afforded 2-amino-5-(methylsulfonamidomethyl)benzamide (0.035 g, 53%).

A mixture of 2-amino-5-(methylsulfonamidomethyl)benzamide (0.035 g, 0.14 mmol), 4-hydroxy-3,5-dimethyl benzaldehyde (0.022 g, 0.14 mmol) and CuCl₂ (0.039 g, 0.28 mmol) in EtOH (5 mL) was refluxed for 3 h, then concentrated in vacuo. Purification by flash chromatography on silica gel, eluting with 92/7/1 CHCl₃:MeOH:concentrated NH₄OH, followed by reverse-phase chromatography, eluting with 10% to 50% CH₃CN in H₂O with 0.1% TFA, and finally flash chromatography on silica gel, eluting with 7:2.5:0.5 CHCl₃/MeOH/concentrated NH₄OH, afforded the title compound (0.030 g, 57%) as a white solid. ¹H NMR (300 MHz, DMSO-d₆): δ 8.09 (s, 1H), 7.83-7.90 (m, 2H), 7.65-7.78 (m, 3H), 6.81-7.54 (m, 2H), 4.30 (d, J=6.2 Hz, 2H), 2.91 (s, 3H), 2.24 (s, 6H). ESI MS m/z 374 [M+H]⁺.

### Example 12

### Preparation of 2-(4-(2-(benzyloxy)ethoxy)-3,5-dimethylphenyl)-5,7-dimethoxypyrido[2,3-d]pyrimidin-4(3H)-one

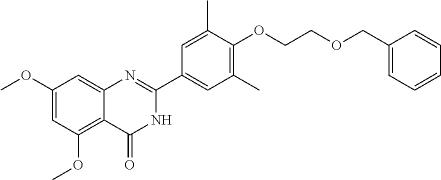 [SMILES: COc1cc(OC)c2c(=O)[nH]c(-c3cc(C)c(OCCOCc4ccccc4)c(C)c3)nc2c1]

A mixture of dimethyl acetone-1,3-dicarboxylate (200 g 1.148 mol), cyanamide (48.3 g, 1.148 mol), and Ni(acac)₂ (14.75 g, 0.0574 mol) in dioxane (200 mL) was heated to reflux in a 1-L flask with a reflux condenser. The reaction mixture was heated at reflux for 16 hours and then cooled to room temperature. The precipitate was filtered off, and the solid was mixed with methanol (200 mL), stirred for 30 minutes, and filtered again to give methyl 2-amino-4-hydroxy-6-oxo-1,6-dihydropyridine-3-carboxylate (93 g, 44%).

In a 1-L flask with a reflux condenser was added methyl 2-amino-4-hydroxy-6-oxo-1,6-dihydropyridine-3-carboxylate (93.0 g, 0.505 mol) and POCl₃ (425 mL) and the reaction mixture was heated to reflux for 35 minutes. About 300 mL POCl₃ was evaporated under vacuum. The residue was poured into ice and water (400 mL), which was further neutralized with KOH to pH approximately 6-7. The precipitate was filtered off and extracted with ethyl acetate (2×300 mL). The organic solution was concentrated and passed through a column, eluting with hexane:ethyl acetate 4:1, to give methyl 2-amino-4,6-dichloropyridine-3-carboxylate (22.5 g, 20.1%).

In a 500-mL flask with a reflux condenser was added methyl 2-amino-4.6-dichloropyridine-3-carboxylate (22.5 g, 0.101 mol) and 25 wt % sodium methoxide in methanol (88 mL, 0.407 mol), together with methanol (20 mL). The mixture was heated to reflux for 5 hours, then cooled to room temperature. Acetic acid (15 mL) was added to the mixture and pH was adjusted to approximately 7. Methanol was removed and the residue was poured into water (100 mL). The precipitated solid was filtered and further rinsed with water (3×200 mL) to give methyl 2-amino-4,6-dimethoxypyridine-3-carboxylate (18.5 g, 86.4%).

In a 500-mL flask with a reflux condenser was added methyl 2-amino-4,6-dimethoxypyridine-3-carboxylate (18.5 g, 0.0872 mol), potassium hydroxide (19.5 g, 0.349 mol) in water (80 mL) and ethanol (100 mL). The mixture was heated to 80° C. for 16 hours. The solvent was removed and aqueous HCl was used to adjust the pH to 6. The water was removed by freeze drying. The obtained solid was extracted with methanol to yield 2-amino-4,6-dimethoxy-nicotinic acid (17.2 g, 100%).

2-Amino-4,6-dimethoxy-nicotinic acid (17.2 g, 0.0872 mol) was added to THF (110 mL). 1-[3-(Dimethylamino)propyl]-3-ethylcarbodiimide hydrochloride (21.73 g, 0.113 mol), 1-hydroxybenzotriazole hydrate (12.96 g, 0.0959 mol) and 4-methyl morpholine (9.7 g, 0.0959 mol) were then added to the suspension. After stirring for 10 minutes at room temperature, 50% v/v ammonium hydroxide (18.3 g, 0.262 mol) was added. The reaction mixture was kept at room temperature for 16 hours. THF was removed and the residue was poured into cold water (100 mL). The precipitate was filtered off and washed with cold water to yield 2-amino-4,6-dimethoxy-nicotinamide (10.8 g, 62.3%).

To a solution of 4-hydroxy-3,5-dimethylbenzaldehyde (6.84 g, 0.0455 mol) in anhydrous DMF (15 mL) was added NaH in mineral oil (60%, 2.23 g, 0.0558 mol). (2-Bromo-ethyoxymethyl)-benzene (10.0 g, 0.0465 mol) was added and the reaction was kept at 65° C. overnight. The reaction mixture was poured into water and extracted with dichloromethane to yield (4-(2-benzyloxy-ethoxy)-3,5-dimethylbenzaldehyde (10.5 g, 81%), which was used for next step reaction without further purification.

To a solution of 2-amino-4,6-dimethoxy-nicotinamide (2.55 g, 12.9 mmol) and 4-(2-benzyloxy-ethoxy)-3,5-dimethylbenzaldehyde (3.68 g, 12.9 mmol) in N,N-dimethyl acetamide (20 mL), were added NaHSO₃ (2.52 g, 14.2 mmol) and p-TSA (1.98 g, 10.4 mmol). The reaction mixture was heated at 150° C. for 14 hours. The reaction mixture was cooled to room temperature. The solvent was removed under reduced pressure. The residue was diluted with water and the solid was collected and further washed with methanol. The crude product was purified by column chromatography (silica gel 230-400 mesh; 2% methanol in CH₂Cl₂ as eluent) to give the title compound as an off-white solid (0.88 g, 14.7%). MP 204.5-205.9° C.

### Example 13

### Preparation of 2-(4-(2-hydroxyethoxy)-3,5-dimethylphenyl)-5,7-dimethylpyrido[2,3-d]pyrimidin-4(3H)-one

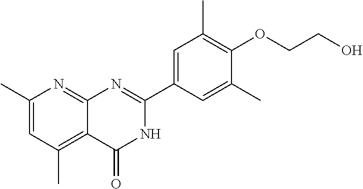 [SMILES: Cc1cc(C)c2c(=O)[nH]c(-c3cc(C)c(OCCO)c(C)c3)nc2n1]

A mixture of 3,5-dimethoxy-4-hydroxybenzaldehyde (10 g, 67 mmol), (2-bromoethoxy)-dimethyl-tert-butylsilane (15 mL, 70 mmol), potassium iodide (1.1 g, 6.7 mmol), and sodium hydride (4 g, 100 mmol) in DMF (150 mL) was heated and stirred at 70° C. for 14 hours. The reaction was then cooled and quenched by adding water (100 mL). The mixture was extracted with EtOAc (3×100 mL) and concentrated on a rotary evaporator. The resulting residue was purified by column (SiO₂, hexanes/EtOAc=6:1) to yield 4-[2-(tert-butyl-dimethyl-silanyloxy)-ethoxy]-3,5-dimethyl-benzaldehyde (15.4 g, 75%).

A mixture of 2-amino-4,6-dimethyl-nicotinamide (0.25 g, 1.5 mmol), 4-[2-(tert-butyl-dimethyl-silanyloxy)-ethoxy]-3,5-dimethyl-benzaldehyde (0.468 g, 1.5 mmol), sodium hydrogensulfite (0.271 g, 1.51 mmol) and p-toluenesulfonic acid (0.358 g, 1.82 mmol) in N,N-dimethyl acetamide (10 mL) was stirred at 150° C. for 4 hours. The reaction mixture was cooled to room temperature, diluted with water (50 mL), basified with sodium bicarbonate, to pH approximately 8-9, extracted with EtOAc (3×100 mL), and concentrated on a rotary evaporator, to afford a solid residue, which was purified by column chromatography (SiO₂, DCM/MeOH/EtOAc=6:1:2) to yield 2-{4-[2-(tert-butyl-dimethyl-silanyloxy)-ethoxy]-3,5-dimethyl-phenyl}-5,7-dimethyl-3H-pyrido[2,3-d]pyrimidin-4-one (56 mg, 8%).

To a solution of 2-{4-[2-(tert-butyl-dimethyl-silanyloxy)-ethoxy]-3,5-dimethyl-phenyl}-5,7-dimethyl-3H-pyrido[2,3-d]pyrimidin-4-one (107 mg, 0.234 mmol) in THF (10 mL) was added TBAF in THF (3 mL, 3 mmol) and the mixture was stirred at room temperature for 15 hours. The mixture was then concentratred on a rotary evaporator and subjected to column chromatography (SiO₂, DCM/MeOH/EtOAc=6:1:2) to yield 2-[4-(2-hydroxy-ethoxy)-3,5-dimethyl-phenyl]-5,7-dimethyl-3H-pyrido[2,3-d]pyrimidin-4-one (36 mg, 45%).

A solution of 2-[4-(2-hydroxy-ethoxy)-3,5-dimethyl-phenyl]-5,7-dimethyl-3H-pyrido-[2,3-d]pyrimidin-4-one (36 mg, 0.105 mmol) in MeOH (5 mL) and DCM (5 mL) was mixed with HCl in ether (2 mL, 2 mmol) and stirred at room temperature for 30 minutes. The reaction mixture was then concentrated on a rotary evaporator. The resulting solid residue was re-dissolved in minimal volume of MeOH-DCM (1:1) and triturated with hexanes. The solid was collected by filtration and washed with MeOH-DCM (1:20) to yield the title compound as a yellow solid (16.6 mg, 41%).

### Example 14

### Preparation of 5,7-difluoro-2-(4-(2-hydroxyethoxy)-3,5-dimethylphenyl)quinazolin-4(3H)-one

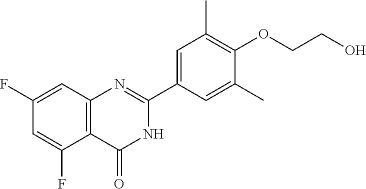 [SMILES: Cc1cc(-c2nc3cc(F)cc(F)c3c(=O)[nH]2)cc(C)c1OCCO]

A mixture of 3,5-dimethoxy-4-hydroxybenzaldehyde (10 g, 66.67 mmol), (2-bromoethoxy)-dimethyl-tert-butylsilane (15 mL, 70 mmol), potassium iodide (1.1 g, 6.67 mmol), and sodium hydride (4.00 g, 100 mmol) in DMF (150 mL) was heated and stirred at 70° C. for 14 hours. The reaction was then cooled and quenched by addition of water (100 mL). The mixture was extracted with EtOAc (3×100 mL) and concentrated on a rotary evaporator. The resulting residue was purified by column (SiO₂, hexanes/EtOAc=6:1) to yield 4-[2-(tert-butyl-dimethyl-silanyloxy)-ethoxy]-3,5-dimethyl-benzaldehyde (15.4 g, 75%).

A solution of 2-amino-4,6-difluorobenzoic acid (0.5 g, 2.9 mmol), EDCl HCl (0.887 g, 4.62 mmol), HOBt (0.975 g, 7.22 mmol), and triethylamine (1.6 mL, 11.552 mmol) in THF (50 mL) was stirred at room temperature for 1 hour. Ammonium hydroxide (50% aqueous, 10 mL) was then added to the reaction mixture. The resulting mixture was stirred at room temperature for 6 hours. The reaction was quenched by adding water (50 mL), extracted with DCM (3×100 mL), and concentrated on a rotary evaporator to afford 2-amino-4,6-difluorobenzamide (0.25 g, 50%).

A mixture of 2-amino-4,6-difluoro benzamide (0.25 g, 1.45 mmol), 4-[2-(tert-butyl-dimethyl-silanyloxy)-ethoxy]-3,5-dimethyl-benzaldehyde (0.448 g, 1.45 mmol), sodium hydrogensulfite (0.26 g, 1.45 mmol) and p-toluenesulfonic acid (0.276 g, 1.45 mmol) in N,N-dimethyl acetamide (10 mL) was stirred at 155° C. for 14 hours. The reaction mixture was cooled to room temperature, diluted with water (50 mL), extracted with EtOAc (3×100 mL), and concentrated on a rotary evaporator, to afford impure product. The residue was re-dissolved in THF (20 mL) and mixed with TBAF in THF (10 mL, 10 mmol). The reaction mixture was stirred at room temperature for 3 hours and concentrated on a rotary evaporator to afford an oily residue. Further purification by column (SiO₂, EtOAc/DCM=3:1) yielded a light yellow solid. This solid was diluted with MeOH (10 mL) to make a slurry. The solid was collected by filtration and washed with MeOH to afford the title compound as a light yellow solid (49 mg, 5% overall yield).

### Example 15

### Preparation of 2-[4-(2-hydroxy-ethoxy)-3,5-dimethyl-phenyl]-5,7-diisopropoxy-3H-quinazolin-4-one

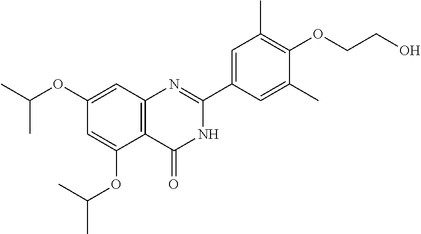 [SMILES: Cc1cc(-c2nc3cc(OC(C)C)cc(OC(C)C)c3c(=O)[nH]2)cc(C)c1OCCO]

To a solution of 3,5-dihydroxybenzoic acid (10.0 g, 64.9 mmol) in anhydrous ethanol (100 mL) at room temperature was slowly added concentrated sulfuric acid (10 mL). The resulting mixture was stirred at reflux for 36 hours. The reaction was cooled to room temperature, diluted with water (200 mL), extracted with CH₂Cl₂ (3×100 mL), and concentrated on a rotary evaporator, to afford 3,5-dihydroxybenzoic acid ethyl ester as a colorless oil. Yield: 8.2 g (69%).

A solution of 3,5-dihydroxybenzoic acid ethyl ester (6.0 g, 33 mmol) and 2-iodo-propane (9.9 mL, 99 mmol) in DMF (200 mL) was mixed with potassium carbonate (13.7 g, 98.9 mmol) and the mixture was stirred at room temperature for 14 hours. The reaction mixture was then diluted with water (300 mL), and extracted with ethyl acetate (3×100 mL). The residue obtained upon concentration was subjected to column chromatography (SiO₂, hexanes/ethyl acetate=3:1) to afford 3,5-diisopropoxybenzoic acid ethyl ester. Yield: 8.80 g (100%).

A solution of 3,5-diisopropoxybenzoic acid ethyl ester (8.80 g, 33.1 mmol) and lithium hydroxide (3.18 g, 132 mmol) in water (100 mL), methanol (50 mL), and THF (50 mL) was stirred at reflux for 3 hours. It was then cooled to room temperature, diluted with water (200 mL), acidified with 2 N hydrochloric acid, to pH approximately 2, extracted with CH₂Cl₂ (3×100 mL), and concentrated on a rotary evaporator, to afford 3,5-diisopropoxybenzoic acid as a white solid. Yield: 7.60 g (97%).

A solution of 3,5-diisopropoxybenzoic acid (7.60 g, 31.9 mmol), triethylamine (5.3 mL, 38 mmol), and diphenylphosphoroyl azide (8.3 mL, 38 mmol) in 1,4-dioxane (120 mL) and tert-butanol (30 mL) was stirred at reflux for 16 hours. The reaction mixture was then cooled to room temperature, diluted with 0.2 N sodium bicarbonate aqueous (200 mL), extracted with CH₂Cl₂ (3×100 mL), and concentrated on a rotary evaporator. The residue obtained was subjected to column chromatography (SiO₂, hexanes/ethyl acetate=3:1) to afford 3,5-diisopropoxyphenyl)-carbamic acid tert-butyl ester as a white solid. Yield: 5.60 g (57%).

A solution of 3,5-diisopropoxyphenyl)-carbamic acid tert-butyl ester (5.60 g, 18.2 mmol) in trifluoroacetic acid (30 mL) was stirred at reflux for 30 minutes and concentrated on a rotary evaporator to dryness to afford 3,5-diisopropoxyphenylamine trifluoroacetic acid salt as an oil. Yield: 5.27 g (90%).

To a round-bottomed flask contained 3,5-diisopropoxyphenylamine trifluoroacetic acid salt (5.27 g, 16.4 mmol) was slowly added oxalyl chloride (20 mL) and the mixture was stirred at reflux for 1 hour. Extra oxalyl chloride was removed by distillation and methanol (100 mL) was added to the residue. It was then stirred at room temperature for 30 minutes and concentrated to dryness on a rotary evaporator to afford 4,6-diisopropoxy-1H-indole-2,3-dione as a semi-solid. Yield: 4.33 g (100%).

A solution of potassium hydroxide (15.3 g, 273 mmol) in water (60 mL) was mixed with 4,6-diisopropoxy-1H-indole-2,3-dione (4.33 g, 16.4 mmol). To this mixture was slowly added hydrogen peroxide. The resulting mixture was stirred at 70° C. for 30 minutes and cooled to 0° C. The mixture was acidified at 0° C. with 2 N hydrochloric acid to pH approximately 4, extracted with CH₂Cl₂ (3×100 mL), and concentrated on a rotary evaporator to afford 2-amino-4,6-diisopropoxy-benzoic acid as a semi-solid. Yield: 2.91 g (70%).

A solution of 2-amino-4,6-diisopropoxybenzoic acid (2.91 g, 11.5 mmol), N-(3-dimethylaminopropyl)-N′-ethylcarbodiimide hydrochloride (3.20 g, 16.7 mmol), HOBt (3.10 g, 23.0 mmol), and triethylamine (4.2 mL, 30 mmol) in THF (200 mL) was stirred at room temperature for 20 minutes. 50% (v/v) ammonia aqueous (20 mL) was then added. The resulting solution was stirred at room temperature for 14 hours, diluted with water (200 mL), extracted with CH₂Cl₂ (3×100 mL), and concentrated on a rotary evaporator. The residue obtained was subjected to column chromatography (SiO₂, ethyl acetate/dichloromethane/methanol=6:2:1) to afford 2-amino-4,6-diisopropoxybenzamide. Yield: 1.2 g (41%).

A solution of 2-amino-4,6-diisopropoxybenzamide (0.30 g, 1.2 mmol), 4-(2-hydroxy-ethoxy)-3,5-dimethylbenzaldehyde (0.28 g, 1.4 mmol), sodium bisulfite (0.25 g, 1.4 mmol), and p-toluenesulfonic acid (20 mg, 0.11 mmol) in dimethyl acetamide (10 mL) was stirred at 150° C. for 12 hours. Extra solvent was evaporated on a rotary evaporator and the residue was diluted with saturated sodium bicarbonate aqueous solution (100 mL) and extracted with CH₂Cl₂ (3×100 mL). The residue obtained upon concentration was subjected to column chromatography (SiO₂, ethyl acetate/dichloromethane/hexanes/methanol=4:4:4:1) to afford the title compound as a light yellow solid. Yield: 35 mg (6.9%). ¹H NMR (400 MHz, CDCl₃): δ 9.78 (br s, 1H), 7.66 (s, 2H), 6.78 (d, 1H), 6.42 (d, 1H), 4.72 (m, 1H), 4.63 (m, 1H), 3.97 (t, 3H), 3.92 (t, 2H), 2.33 (s, 6H), 1.45 (d, 3H), 1.41 (d, 3H). MS (ES⁺) m/z: 427.13 (M+1).

### Example 16

### Preparation of 2-[4-(2,3-Dihydroxy-propoxy)-3,5-dimethyl-phenyl]-5,7-dimethoxy-3H-quinazolin-4-one

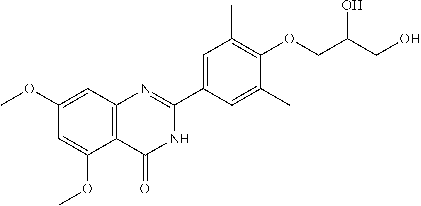 [SMILES: COc1cc(OC)c2c(=O)[nH]c(-c3cc(C)c(OCC(O)CO)c(C)c3)nc2c1]

To a solution of 4-hydroxy-3,5-dimethylbenzaldehyde (1.50 g, 10.0 mmol) in anhydrous DMF (20 mL) were added cesium carbonate (6.52 g, 20.0 mmol) and 4-chloromethyl-2,2-dimethyl-[1,3]dioxolane (1.50 g, 10.0 mmol). The reaction mixture was stirred at 80° C. for 4 days under nitrogen, then cooled to room temperature. Water (100 mL) was added, and the mixture extracted with ethyl acetate (200 mL). The organic phase was separated, washed with 1 N aqueous NaOH solution (100 mL), water (2×100 mL), brine (100 mL), and dried over anhydrous Na₂SO₄. Solvent was removed under reduced pressure, and the crude compound was purified using the Simpliflash system (20% ethyl acetate in hexanes as eluent) to give 4-(2,2-dimethyl-[1,3]dioxolane-4-ylmethoxy)-3,5-dimethyl-benzaldehyde as a yellow oil. Yield: 0.95 g (36%).

To a solution of 2-amino-4,6-dimethoxybenzamide (0.35 g, 1.8 mmol) in N,N-dimethyl acetamide (10 mL) were added 4-(2,2-dimethyl-[1,3]dioxolane-4-ylmethoxy)-3,5-dimethyl-benzaldehyde (0.520 g, 1.98 mmol), sodium hydrogensulfite (58.5 wt %) (0.350 g, 1.98 mmol) and p-toluenesulfonic acid (0.17 g, 0.90 mmol). The reaction mixture was stirred at 120° C. for 16 hours under nitrogen, then cooled to room temperature. Solvent was evaporated under reduced pressure, water (50 mL) was added, the separated solid was filtered, washed with water, then dichloromethane (10 mL), and dried under vacuum to give the title compound as a yellow solid. Yield: 0.34 g (47%). ¹H NMR (400 MHz, DMSO-d₆): δ 11.8 (s, 1H), 7.83 (s, 2H), 6.64 (s, 1H), 6.44 (s, 1H), 4.95 (d, 1H), 4.40 (t, 1H), 3.88 (s, 3H), 3.84-3.66 (m, 6H), 3.46 (t, 2H), 2.28 (s, 6H). MS (ES) m/z: 401.04 (M+1) (100%).

### Example 17

### Preparation of 2-[4-(2-hydroxy-ethoxy)-phenyl]-5,7-dimethoxy-3H-quinazolin-4-one

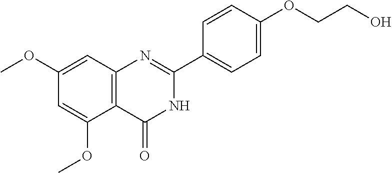 [SMILES: COc1cc(OC)c2c(=O)[nH]c(-c3ccc(OCCO)cc3)nc2c1]

To a flask (250 mL) with a magnetic stirrer were added 4-hydroxybenzalde (10.0 g, 81.8 mmol), 2-chloroethanol (26.3 g, 327 mmol), potassium carbonate (22.6 g, 163 mmol), and ethanol (80 mL). The reaction mixture was stirred at 70° C. for 16 hours. Potassium carbonate was filtered and ethanol was removed. The residue was diluted with ethyl acetate (200 mL) and washed with 5% sodium hydroxide (100 mL), water (100 mL), and brine (100 mL). The crude product was purified by column chromatography (silica gel, 230-400 mesh), using hexane/ethyl acetate (1:1) as eluent, to afford 4-(2-hydroxy-ethoxy)-benzaldehyde. Yield: 10.0 g (73%).

To a solution of 2-amino-4,6-dimethoxy-benzamide (0.400 g, 2.00 mmol) and 4-(2-hydroxy-ethoxy)-benzaldehyde (0.340 g, 2.00 mmol) in N,N-dimethylacetamide (8 mL) were added NaHSO₃ (0.390 g, 2.20 mmol) and p-TSA (38 mg, 0.20 mmol). The reaction mixture was stirred at 115-120° C. for 5 hours and cooled to room temperature. The solvent was removed under reduced pressure. The residue was diluted with water (40 mL) and the solid was collected, mixed with methanol (50 mL), and stirred for 30 min. The solid was filtered and rinsed with ether (30 mL) to give the title compound as white solid. Yield: 0.42 g (61%). ¹H NMR (400 Hz, DMSO-d₆): δ 11.98 (s, 1H), 8.18 (d, 2H), 7.08 (d, 2H), 6.78 (s, 1H), 6.52 (s, 1H), 4.98 (s, 1H), 4.10 (t, 2H), 3.90 (s, 3H), 3.84 (s, 3H), 3.74 (t, 2H). MS (ES⁺) m/z: 343.13 (M+1).

### Example 18

### Preparation of 2-[4-(2-hydroxy-ethoxy)-naphthalen-1-yl]-5,7-dimethoxy-3H-quinazolin-4-one

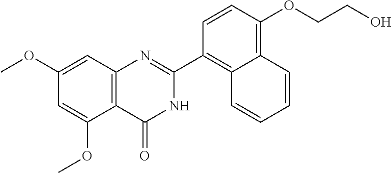 [SMILES: COc1cc(OC)c2c(=O)[nH]c(-c3ccc(OCCO)c4ccccc34)nc2c1]

To a mixture of 4-hydroxy-naphthalene-1-carbaldehyde (1.0 g, 5.8 mmol) and potassium carbonate (2.40 g, 17.4 mmol) in N,N-dimethylformamide (3 mL) under nitrogen was added 2-chloroethanol (0.80 mL, 12 mmol). The reaction mixture was heated at reflux for 20 hours and the solvent was then removed under reduced pressure. The residue was diluted with ethyl acetate, washed with water, 0.2 N aqueous sodium hydroxide, brine, and dried over anhydrous sodium sulfate. The crude oil (1.03 g) was purified by column chromatography (silica gel 230-400 mesh; methylene chloride/EtOAc=3/7), to give 4-(2-hydroxy-ethoxy)-naphthalene-1-carbaldehyde as a colorless oil. Yield: 0.6 g (48%).

To a solution of 2-amino-4,6-dimethoxy-benzamide (0.45 g, 2.3 mmol) in N,N-dimethylacetamide (25 mL) under nitrogen was added 4-(2-hydroxy-ethoxy)-naphthalene-1-carbaldehyde (0.50 g, 2.3 mmol) followed by sodium hydrogensulfite (0.26 g, 2.5 mmol) and p-toluenesulfonic acid (0.22 g, 1.1 mmol). The resulting mixture was heated at 130° C. for 15 hours and the solvent was removed under reduced pressure. The residue was diluted with ethyl acetate, washed with water, and dried over sodium sulfate. The crude orange solid (0.37 g) was purified by column chromatography (silica gel, 230-400 mesh; 3/7 methylene chloride/EtOAc then 9/1 methylene chloride/MeOH as eluent) and by triturating with methylene chloride and ether to afford the title compound as a light orange solid. Yield: 0.16 g (36%). ¹H NMR (400 MHz, CDCl₃+CD₃OD): δ 8.34 (d, 1H), 8.19 (d, 1H), 7.62 (d, 1H), 7.44-7.53 (m, 2H), 6.84 (d, 1H), 6.75 (s, 1H), 6.43 (s, 1H), 4.22-4.24 (m, 2H), 4.01-4.03 (m, 2H), 9.90 (s, 3H), 3.85 (s, 3H). MS (ES⁺) m/z: 393.27 (M+1).

### Example 19

### Preparation of 2-(2-hydroxymethyl-benzofuran-5-yl)-5,7-dimethoxy-3H-quinazolin-4-one

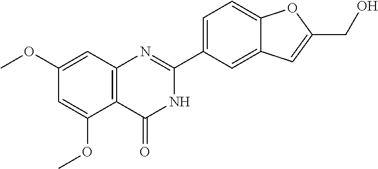 [SMILES: COc1cc(OC)c2c(=O)[nH]c(-c3ccc4oc(CO)cc4c3)nc2c1]

To a solution of 4-hydroxy-benzaldehyde (3.66 g, 30.0 mmol) in 50% (v/v) aqueous ammonium hydroxide (250 mL) was quickly added a solution of potassium iodide (24.9 g, 150 mmol) and iodine (7.62 g, 30.0 mmol) in water (60 mL). The dark colored solution was stirred at room temperature for 1 hour and the color changed to yellow. Stirring was continued at room temperature for 16 hours. The color changed to gray. Then, the reaction mixture was filtered through a celite pad. The filtrate was acidified with concentrated HCl to pH approximately 1 and extracted with ethyl acetate (1×300 mL). The organic phase was washed with water (150 mL) and brine (150 mL), dried over anhydrous Na₂SO₄, and concentrated to give 4-hydroxy-3-iodo-benzaldehyde as an off-white solid (1:1 mixture of starting material and product). Yield: 5.34 g (crude).

To a degassed solution of 4-hydroxy-3-iodo-benzaldehyde (5.34 g, 15.0 mmol) in anhydrous DMF (100 mL) were added bis(triphenylphosphine)palladium(II) dichloride (0.53 g, 0.75 mmol), copper (I) iodide (0.14 g, 0.75 mmol), 1,1,3,3-tetramethyl guanidine (8.64 g, 75.0 mmol), and propargyl alcohol (1.18 g, 21.0 mmol). The reaction mixture was stirred at room temperature for 24 hours under nitrogen and then concentrated to dryness under reduced pressure. The residue was diluted with 2 N aqueous HCl (150 mL) and extracted with ethyl acetate (1×200 mL). Organic phase was washed with water (2×100 mL), brine (100 mL), and dried over anhydrous Na₂SO₄. Solvent was evaporated and crude compound was purified using the Simpliflash system (30% ethyl acetate in hexanes as eluent) to give 2-hydroxymethyl-benzofuran-5-carbaldehyde as a pale yellow solid. Yield: 1.36 g (26% for two steps).

To a solution of 2-hydroxymethyl-benzofuran-5-carbaldehyde (0.450 g, 2.55 mmol) and 2-amino-4,6-dimethoxy-benzamide (0.500 g, 2.55 mmol) in N,N-dimethylacetamide (5 mL) were added sodium hydrogen sulfite (58.5%; 0.510 g, 2.80 mmol) and p-toluenesulfonic acid (50 mg, 0.25 mmol). The reaction mixture was stirred at 120° C. for 6 hours under nitrogen and cooled to room temperature. The separated solid was filtered, washed with ether (30 mL), water (30 mL), and ethyl acetate (20 mL), and then dried under vacuum to give the title compound as a yellow solid. Yield: 0.572 g (64%). ¹H NMR (400 MHz, DMSO-d₆): δ 12.07 (br s, 1H), 8.44 (d, J=2.0 Hz, 1H), 8.10 (dd, J=8.8 and 1.6 Hz, 1H), 7.67 (d, J=8.8 Hz, 1H), 6.89 (s, 1H), 6.76 (d, J=2.4 Hz, 1H), 6.54 (d, J=2.4 Hz, 1H), 4.61 (s, 2H), 3.90 (s, 3H), 3.86 (s, 3H). MS (ES⁺) m/z: 353.20 (M+1).

### Example 20

### Preparation of 7-(2-benzyloxy-ethoxy)-2-[4-(2-hydroxy-ethoxy)-3,5-dimethyl-phenyl]-5-methoxy-3H-quinazolin-4-one

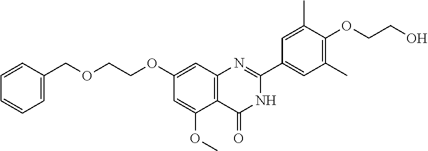 [SMILES: COc1cc(OCCOCc2ccccc2)cc2nc(-c3cc(C)c(OCCO)c(C)c3)[nH]c(=O)c12]

To a solution of 4-hydroxy-3,5-dimethyl-benzaldehyde (1.00 g, 6.70 mmol) in DMF (20 mL) was added cesium carbonate (8.70 g, 26.6 mmol) followed by (2-bromo-ethoxy)-tert-butyl-dimethyl-silane (2.9 mL, 13 mmol). The reaction mixture was stirred at room temperature for 16 hours. Water was added and the product was extracted with ethyl acetate. The solvent was evaporated in vacuo to obtain 4-[2-(tert-butyl-dimethyl-silanyloxy)-ethoxy]-3,5-dimethyl-benzaldehyde as a colorless oil. It was contaminated with (2-bromo-ethoxy)-tert-butyl-dimethyl-silane, but was used in the next step without further purification. Yield: 2.5 g (71%).

To a stirred solution of 2-amino-4,6-difluoro-benzamide (0.50 g, 2.9 mmol) and 4-[2-(tert-butyl-dimethyl-silanyloxy)-ethoxy]-3,5-dimethyl-benzaldehyde (1.3 g, 2.9 mmol) in N,N-dimethylacetamide (10 mL) were added sodium hydrogen sulfite (0.60 g, 3.5 mmol) and p-toluenesulfonic acid (0.1 g, 0.6 mmol) and the reaction mixture was stirred at 120° C. for 16 hours. The solvent was evaporated in vacuo, water was added, and the precipitated solid was filtered off to obtain 2-{4-[2-(tert-butyl-dimethyl-silanyloxy)-ethoxy]-3,5-dimethyl-phenyl}-5,7-difluoro-3H-quinazolin-4-one as a yellow solid, which was used in the next step without further purification. Yield: 0.490 g (36%).

To a suspension of 2-{4-[2-(tert-butyl-dimethyl-silanyloxy)-ethoxy]-3,5-dimethyl-phenyl}-5,7-difluoro-3H-quinazolin-4-one (0.490 g, 1.06 mmol) in DMF (3 mL) was added sodium methoxide in methanol (2.3 mL, 11 mmol) and the reaction mixture was stirred at room temperature for 16 hours. Water was added, the mixture was acidified with acetic acid, to pH approximately 4-5, and the precipitated solid was filtered off to obtain 7-fluoro-2-[4-(2-hydroxy-ethoxy)-3,5-dimethyl-phenyl]-5-methoxy-3H-quinazolin-4-one as a white solid. Yield: 0.21 g (55%).

To a solution of 7-fluoro-2-[4-(2-hydroxy-ethoxy)-3,5-dimethyl-phenyl]-5-methoxy-3H-quinazolin-4-one (0.21 g, 0.59 mmol) in THF (12 mL) was added imidazole (80 mg, 1.2 mmol), followed by tert-butyldiphenylsilyl chloride (0.20 mL, 0.65 mmol). The reaction mixture was stirred at room temperature for 16 hours. Saturated NH₄Cl aqueous solution was added and the product was extracted with ethyl acetate. The solvent was evaporated in vacuo and the residue was purified by column chromatography (silica gel; 230-400 mesh; eluting with 5-10% ethyl acetate/CH₂Cl₂) to afford 2-{4-[2-(tert-butyl-diphenyl-silanyloxy)-ethoxy]-3,5-dimethyl-phenyl}-7-fluoro-5-methoxy-3H-quinazolin-4-one. Yield: 0.36 g (quantitative).

To a solution of 2-benzyloxy-ethanol (3 mL) in dimethyl sulfoxide (3 mL) was added sodium hydride (0.24 g, 6.0 mmol) in portions and the reaction mixture was stirred at room temperature for 45 minutes. To this mixture was added 2-{4-[2-(tert-butyl-diphenyl-silanyloxy)-ethoxy]-3,5-dimethyl-phenyl}-7-fluoro-5-methoxy-3H-quinazolin-4-one (0.36 g, 0.60 mmol) and the reaction mixture was heated at 70° C. for 16 hours. Water was added, and the mixture was acidified with acetic acid, to pH approximately 4-5, and the precipitated solid was filtered off to obtain a crude product, which was purified by preparative HPLC to obtain the title compound as a white solid. Yield: 0.12 g (42%). ¹H NMR (400 MHz, DMSO-d₆): δ11.83 (s, 1H), 7.89 (s, 2H), 7.37 (m, 5H), 6.75 (s, 1H), 6.53 (s, 1H), 4.91 (s, 1H), 4.58 (s, 2H), 4.30 (s, 2H), 3.84-3.73 (m, 9H), 2.31 (s, 6H). MS (ES⁺) m/z: 491.55 (M+1).

### Example 21

### Preparation of 7-(2-benzyloxy-ethoxy)-2-(2-hydroxymethyl-benzofuran-5-yl)-5-methoxy-3H-quinazolin-4-one

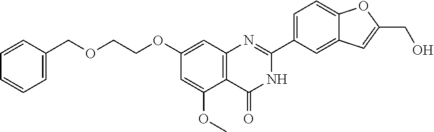 [SMILES: COc1cc(OCCOCc2ccccc2)cc2nc(-c3ccc4oc(CO)cc4c3)[nH]c(=O)c12]

To a stirred solution of 2-hydroxymethyl-benzofuran-5-carbaldehyde (2.00 g, 11.4 mmol) in anhydrous CH₂Cl₂ (25 mL) were added N,N-diisopropylethyl amine (5.17 g, 40.0 mmol) and chloromethyl methyl ether (2.76 g, 34.3 mmol) at room temperature. The reaction mixture was stirred at room temperature for 16 hours under nitrogen. Phosphate buffer (pH 7, 100 mL) was added and the mixture was extracted with dichloromethane (100 mL). The organic phase was separated, washed with brine, and dried over anhydrous Na₂SO₄. Removal of solvent gave 2-methoxymethoxymethyl-benzofuran-5-carbaldehyde as an orange oil. Yield 2.41 g (96%).

To a solution of 2-methoxymethoxymethyl-benzofuran-5-carbaldehyde (2.31 g, 10.5 mmol) and 2-amino-4,6-difluoro-benzamide (1.20 g, 7.00 mmol) in N,N-dimethyl acetamide (15 mL) were added sodium hydrogen sulfite (58.5 wt %; 1.54 g, 8.40 mmol) and p-toluenesulfonic acid monohydrate (0.26 g, 1.40 mmol). The reaction mixture was stirred at 120° C. for 4 hours under nitrogen, then cooled to room temperature. Solvent was evaporated under reduced pressure and water (100 mL) was added. The separated solid was filtered, washed with water (50 mL), and dried under vacuum, to give 5,7-difluoro-2-(2-methoxymethoxymethyl-benzofuran-5-yl)-3H-quinazolin-4-one as a white solid. Yield 0.96 g (37%).

To a suspension of 5,7-difluoro-2-(2-methoxymethoxymethyl-benzofuran-5-yl)-3H-quinazolin-4-one (0.95 g, 2.56 mmol) in anhydrous DMF (5 mL) was added a solution of sodium methoxide (25 wt %) in methanol at 0° C. under nitrogen. Then, the reaction mixture was stirred at 0° C. for 6 hours. Water (20 mL) was added, the mixture was acidified to pH approximately 6 with glacial acetic acid. The separated solid was filtered, washed with water (20 mL), and dried under vacuum to give 7-fluoro-5-methoxy-2-(2-methoxymethoxymethyl-benzofuran-5-yl)-3H-quinazolin-4-one as a white solid. Yield 0.94 g (95%).

Sodium hydride (60% suspension in mineral oil; 0.48 g, 12.0 mmol) was taken in anhydrous DMF (5 mL). 2-Benzyloxyethanol (3.65 g, 24.0 mmol) was added dropwise at room temperature under nitrogen. After the addition, the reaction mixture was stirred at room temperature for 30 minutes. Then, 7-fluoro-5-methoxy-2-(2-methoxymethoxymethyl-benzofuran-5-yl)-3H-quinazolin-4-one (0.46 g, 1.2 mmol) was added and the reaction mixture was stirred at 80° C. for 16 hours. The reaction mixture was then cooled to room temperature. Water (50 mL) was added, the mixture was acidified to pH approximately 6 with glacial acetic acid and extracted with CH₂Cl₂ (2×100 mL). The organic phase was washed with brine (100 mL) and then dried over anhydrous Na₂SO₄. Removal of solvent, followed by purification, by the Simpliflash system (0-2% methanol in CH₂Cl₂ as eluent) gave 7-(2-benzyloxy-ethoxy)-5-methoxy-2-(2-methoxymethoxymethyl-benzofuran-5-yl)-3H-quinazolin-4-one as a white solid. Yield 0.28 g (45%).

To a solution of 7-(2-benzyloxy-ethoxy)-5-methoxy-2-(2-methoxymethoxymethyl-benzo-furan-5-yl)-3H-quinazolin-4-one (0.27 g, 0.53 mmol) in 50% aqueous acetic acid (15 mL), conc. H₂SO₄ (0.3 mL) was added. The reaction mixture was stirred at 75° C. for 2 hours, then cooled to room temperature. Water (50 mL) was added, and the mixture was neutralized to pH approximately 7 with 4 N aqueous NaOH solution. The separated solid was filtered, washed with water (20 mL), and dried under vacuum. Crude compound was purified by column chromatography (silica gel 230-400 mesh; 2:20:78 methanol/ethyl acetate/CH₂Cl₂ as eluent) to give the title compound as a white solid. Yield 0.13 g (52%).

¹H NMR (400 MHz, DMSO-d₆): δ 12.03 (bs, 1H), 8.43 (s, 1H), 8.09 (dd, J=8.58 and 1.95 Hz, 1H), 7.65 (d, J=8.58 Hz, 1H), 7.37-7.29 (m, 5H), 6.88 (s, 1H), 6.77 (d, J=1.95 Hz, 1H), 6.55 (d, J=1.56 Hz, 1H), 5.51 (s, 1H), 4.60 (t, J=4.68 Hz, 4H), 4.31 (s, 2H), 3.90-3.83 (m, 5H). MS (ES+) m/z 473.48 (100%).

### Example 22

### Preparation of 2-[4-(5,7-dimethoxy-4-oxo-3,4-dihydro-quinazolin-2-yl)-2,6-dimethyl-phenoxy]-N-methyl-acetamide

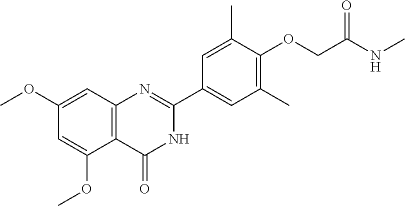

To a solution of [4-(5,7-dimethoxy-4-oxo-3,4-dihydro-quinazolin-2-yl)-2,6-dimethyl-phenoxy]-acetic acid (0.20 g, 0.52 mmol) in anhydrous DMF (8 mL) were added EDCI (0.12 g, 0.62 mmol) and HOBt (0.084 g, 0.62 mmol). Then, a solution of N-methyl amine (2.0 M solution in THF, 1.3 mL, 2.60 mmol) was added and the reaction mixture was stirred at room temperature for 16 hours under nitrogen. Solvent was evaporated under reduced pressure, water (20 mL) was added, and the separated solid was filtered, washed with water (30 mL), ether (20 mL) and dried under vacuum to give the title compound as a white solid. Yield: 0.13 g (63%). ¹H NMR (400 MHz, DMSO-d₆): δ 11.86 (br s, 1H), 8.19 (br s, 1H), 7.91 (s, 2H), 6.74 (d, J=1.95 Hz, 1H), 6.52 (d, J=1.95 Hz, 1H), 4.26 (s, 2H), 3.89 (s, 3H), 3.85 (s, 3H), 2.72 (d, J=4.30 Hz, 3H), 2.30 (s, 6H). MS (ES) m/z: 398.53 (M+1) (100%).

### Example 23

### Preparation of 2-[4-(5,7-Dimethoxy-4-oxo-3,4-dihydro-quinazolin-2-yl)-2,6-dimethyl-phenoxy]-N-(4-methoxy-phenyl)-acetamide

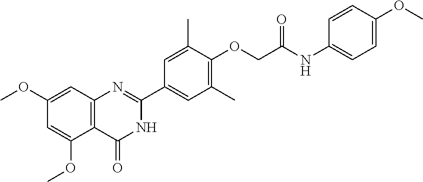

To a solution of 4-hydroxy-3,5-dimethyl-benzaldehyde (9.00 g, 60.0 mmol) in ethanol (300 mL) were added potassium carbonate (24.9 g, 180 mmol) and methyl bromoacetate (11.4 mL, 120 mmol). The reaction mixture was stirred at 95° C. under nitrogen for 16 hours. The mixture was concentrated to dryness under reduced pressure. Water (150 mL) and 1 N NaOH solution (90 mL) were added to the residue. The mixture was stirred at room temperature for 30 minutes, then washed with ether. Concentrated HCl was added slowly to the aqueous solution until a large amount of white precipitate formed. The solid was filtered, washed with water, and air-dried, to give (4-formyl-2,6-dimethyl-phenoxy)-acetic acid as a white solid. Yield: 11.1 g (89%).

To a solution of (4-formyl-2,6-dimethyl-phenoxy)-acetic acid (3.12 g, 15.0 mmol) and 2-amino-4,6-dimethoxy-benzamide (2.94 g, 15.0 mmol) in N,N-dimethylacetamide (50 mL) were added sodium hydrogen sulfite (58.5 wt %, 3.02 g, 16.5 mmol) and p-toluenesulfonic acid monohydrate (0.285 g, 1.50 mmol). The reaction mixture was stirred at 120° C. for 17 hours under nitrogen and cooled to room temperature. The precipitate was filtered, washed with water, then methanol, and air-dried to give 1.29 g [4-(5,7-dimethoxy-4-oxo-3,4-dihydro-quinazolin-2-yl)-2,6-dimethyl-phenoxy]-acetic acid. The filtrate was concentrated to dryness and water was added. The suspension was stirred for 30 minutes and filtered. The solid was washed with water, then methanol. After air drying, 3.78 g more [4-(5,7-dimethoxy-4-oxo-3,4-dihydro-quinazolin-2-yl)-2,6-dimethyl-phenoxy]-acetic acid was obtained. Yield: 5.07 g (88%).

To a mixture of [4-(5,7-dimethoxy-4-oxo-3,4-dihydro-quinazolin-2-yl)-2,6-dimethyl-phenoxy]-acetic acid (0.400 g, 1.04 mmol), 1-ethyl-3-(3′-dimethylaminopropyl)carbodiimide hydrochloride (EDCI; 0.240 g, 1.24 mmol), 1-hydroxybenzotriazole hydrate (HOBt; 0.17 g, 1.24 mmol) in DMF (10 mL) was added 4-methylmorpholine (0.20 mL, 1.8 mmol). After 10 minutes, p-anisidine (0.26 g, 2.08 mmol) was added. The mixture was stirred at room temperature under nitrogen for 2.5 days. The solvent was removed under reduced pressure. Water was added, stirred for 30 minutes. The solid was filtered, washed with water, and dried in air. The crude product was purified by column chromatography (silica gel, 230-400 mesh; 5% MeOH in CH₂Cl₂ as eluent). The product fractions were combined, concentrated to dryness. The solid was dissolved in small amount of dichloromethane, precipitate out by adding ether. The precipitate was filtered, washed with ether, dried under vacuum to afford the title compound as a white solid. Yield: 0.26 g (51%). ¹H NMR (400 MHz, CDCl₃): δ 10.30 (br s, 1H), 8.52 (s, 1H), 7.83 (s, 2H), 7.58 (dd, J=6.8 and 2.0 Hz, 2H), 6.93 (dd, J=6.8 and 2.0 Hz, 2H), 6.84 (d, J=2.4 Hz, 1H), 6.48 (d, J=2.0 Hz, 1H), 4.44 (s, 2H), 3.97 (s, 3H), 3.94 (s, 3H), 3.83 (s, 3H), 2.42 (s, 3H). MS (ES⁺) m/z: 490.55 (M+1).

### Example 24

### Preparation of N-benzyl-2-[4-(5,7-dimethoxy-4-oxo-3,4-dihydroquinazolin-2-yl)-2,6-dimethylphenoxy]acetamide

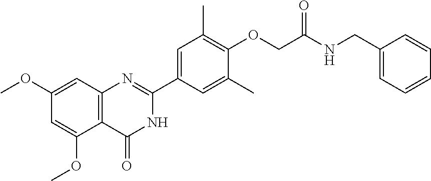 [SMILES: COc1cc(OC)c2c(=O)[nH]c(-c3cc(C)c(OCC(=O)NCc4ccccc4)c(C)c3)nc2c1]

To a mixture of [4-(5,7-dimethoxy-4-oxo-3,4-dihydroquinazolin-2-yl)-2,6-dimethylphenoxy]acetic acid (0.25 g, 0.65 mmol), 1-ethyl-3-(3′-dimethylaminopropyl) carbodiimide hydrochloride (EDCI; 0.137 g, 0.715 mmol), 1-hydroxybenzotriazole hydrate (HOBT; 0.110 g, 0.715 mmol) in DMF (3 mL) was added 4-methylmorpholine (0.08 mL, 0.715 mmol) at room temperature. After 10 minutes, benzylamine (0.142 mL, 1.30 mmol) was added. The mixture was stirred at room temperature under nitrogen for 15 hours. The solvent was removed under reduced pressure. The crude compound was purified by column chromatography (silica gel 230-400 mesh; 3% methanol in dichloromethane as eluent), followed by triturating with an ether-hexane mixture to afford the title compound as a white solid. Yield: 60 mg (39%). ¹H NMR (400 MHz, DMSO-d₆): δ 11.86 (s, 1H), 8.79 (t, J=6.2 Hz, 1H), 7.89 (s, 2H), 7.34-7.21 (m, 5H), 6.72 (d, J=2.0 Hz, 1H), 6.50 (d, J=2.0 Hz, 1H), 4.38 (d, J=6.0 Hz, 2H), 4.33 (s, 2H), 3.87 (s, 3H), 3.82 (s, 3H), 2.30 (s, 6H). MS (ES⁺) m/z: 474.49 (M+1).

### Example 25

### Preparation of 2-[4-(4-hydroxy-butoxy)-3,5-dimethyl-phenyl]-5,7-dimethoxy-3H-quinazolin-4-one

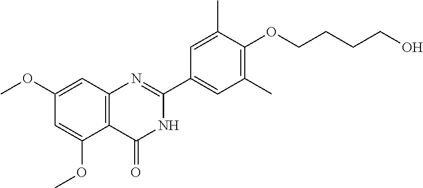 [SMILES: COc1cc(OC)c2c(=O)[nH]c(-c3cc(C)c(OCCCCO)c(C)c3)nc2c1]

To a solution of 4-hydroxy-3,5-dimethyl benzaldehyde (5.00 g, 33.3 mmol) in DMF (30 mL) were added 4-bromo butan-1-ol (6.11 g, 39.9 mmol) and Cs₂CO₃ (16.24 g, 50.0 mmol). The reaction mixture was stirred at room temperature for 48 hours. Water was added and the products were extracted with ethyl acetate (2×200 mL). The combined organic phase was washed with water (100 mL), brine (100 mL), and dried over anhydrous Na₂SO₄. Solvent was removed and the crude compound was purified using the Simpliflash system (40% ethyl acetate in hexane as eluent) to give 4-(4-hydroxybutoxy)-3,5-dimethyl benzaldehyde as a colorless liquid. Yield: 0.66 g (7%).

To a solution of 2-amino-4,6-dimethoxy-benzamide (0.50 g, 2.53 mmol) and 4-(4-hydroxybutoxy)-3,5-dimethyl benzaldehyde (0.66 g, 2.53 mmol) in N,N-dimethyl acetamide (10 mL), NaHSO₃ (0.50 g, 2.79 mmol) and p-TSA (96 mg, 0.50 mmol) were added and the reaction mixture was heated at 115° C. for 16 hours, then cooled to room temperature. Solvent was removed under reduced pressure. Water (100 mL) was added and the mixture was stirred for 1 hour. The solid separated was filtered and dried. The solid was again washed with diethyl ether to give the title compound as a white solid. Yield: 1.69 g (82%). ¹H NMR (400 MHz, CDCl₃): δ 9.10 (s, 1H), 7.66 (s, 2H), 6.83 (d, J=2.4 Hz, 1H), 6.46 (d, J=2.0 Hz, 1H), 3.98 (s, 3H), 3.93 (s, 3H), 3.85 (t, J=6.0 Hz, 2H), 3.78 (m, 2H), 2.36 (s, 6H), 1.94 (m, 2H), 1.85 (m, 2H). MS (ES) m/z: 399.12 (M+1) (100%).

### Example 26

### Preparation of 7-chloro-2-(4-(2-hydroxyethoxy)-3,5-dimethylphenyl)quinazolin-4(3H)-one

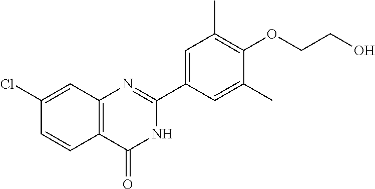 [SMILES: Cc1cc(-c2nc3cc(Cl)ccc3c(=O)[nH]2)cc(C)c1OCCO]

Following the method described in Example 33, the title compound was made starting from 2-amino-4-chlorobenzoic acid and isolated as a white solid. ¹H NMR (300 MHz, DMSO-d₆): δ 12.46 (s, 1H), 8.12 (d, J=8.49 Hz, 1H), 7.90 (s, 2H), 7.77 (d, J=2.00 Hz, 1H), 7.52 (dd, J=8.49, 2.00 Hz, 1H), 4.90 (t, J=5.51 Hz, 1H), 3.86 (t, J=4.88 Hz, 2H), 3.76-3.69 (m, 2H), 2.32 (s, 6H). MS (APCI) m/z 345 [C₁₈H₁₇ClN₂O₃+H]⁺.

### Example 27

### Preparation of 5-chloro-2-(4-(2-hydroxyethoxy)-3,5-dimethylphenyl)quinazolin-4(3H)-one

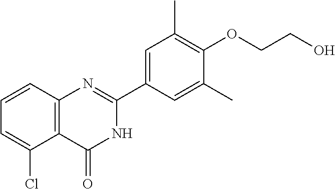 [SMILES: Cc1cc(-c2nc3cccc(Cl)c3c(=O)[nH]2)cc(C)c1OCCO]

A mixture of 2-amino-6-chlorobenzoic acid (5.00 g, 29.1 mmol) in acetonitrile (50.0 mL) was stirred at room temperature under nitrogen. Pyridine (4.72 mL, 58.3 mmol) was added, followed by drop-wise addition of triphosgene (2.85 g, 9.60 mmol) in CH₂Cl₂ (20.0 mL). After the addition, the mixture was heated at 55° C. for 2 hours, then cooled to 25° C. and stirred overnight. Water (100 mL) was added to quench, the mixture was filtered, and washed with cold CH₂Cl₂, to provide 5-chloro-1H-benzo[d][1,3]oxazine-2,4-dione (3.54 g, 62%) as a white solid.

A mixture of 5-chloro-1H-benzo[d][1,3]oxazine-2,4-dione (3.50 g, 17.7 mmol) and 2 M NH₃ in EtOH (11.5 mL, 23.0 mmol) and EtOH (10.0 mL) was stirred at room temperature for 2 hours. The volatiles were removed under reduced pressure, the residue was triturated with water (50 mL), and the solid was filtered, to provide 2-amino-6-chlorobenzamide (1.60 g, 49%) as a tan solid.

A mixture of 2-amino-6-chlorobenzamide (0.490 g, 3.00 mmol), 4-(2-(tert-butyldimethylsilyloxy)ethoxy)-3,5-dimethylbenzaldehyde (0.925 g, 3.00 mmol), NaHSO₃ (94%, 0.468 g, 4.50 mmol), and p-TsOH.H₂O (0.171 g, 0.900 mmol) in DMA (10.0 mL) was heated at 140° C. for 16 hours. The mixture was cooled to room temperature and the solvent was removed under reduced pressure. The residue was diluted with EtOAc (50 mL), washed with water (50 mL), then brine (50 mL), dried over anhydrous Na₂SO₄, filtered, and the solvent was removed under reduced pressure, to provide 2-(4-(2-(tert-butyldimethylsilyloxy)ethoxy)-3,5-dimethylphenyl)-5-chloroquinazolin-4(3H)-one as an off-white solid. The crude material was used directly in the next step without characterization.

Following the method described for desilylation using TBAF in Example 33 below, the title compound was made from 2-(4-(2-(tert-butyldimethylsilyloxy)ethoxy)-3,5-dimethylphenyl)-5-chloroquinazolin-4(3H)-one in 21% yield and was isolated as a white solid. ¹H NMR (300 MHz, DMSO-d₆): δ 12.32 (s, 1H), 7.90 (s, 2H), 7.82-7.55 (m, 2H), 7.48 (dd, J=7.54, 1.35 Hz, 1H), 4.90 (t, J=5.51 Hz, 1H), 3.86 (t, J=4.90 Hz, 2H), 3.77-3.68 (m, 2H), 2.32 (s, 6H). MS (APCI) m/z 345 [C₁₈H₁₇ClN₂O₃+H]⁺.

### Example 28

### Preparation of 2-(4-(2-hydroxyethoxy)-3,5-dimethylphenyl)-7-methoxyquinazolin-4(3H)-one

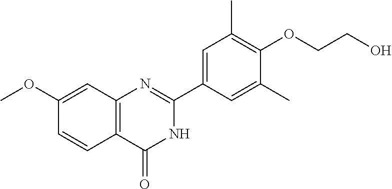 [SMILES: COc1ccc2c(=O)[nH]c(-c3cc(C)c(OCCO)c(C)c3)nc2c1]

A mixture of 2-nitro-4-methoxybenzoic acid (1.00 g, 5.10 mmol) in methanol (10.0 mL) was stirred at room temperature under nitrogen. Palladium on carbon (10% wt, 50% wet, 0.559 g, 0.255 mmol) was added. The round-bottomed flask was capped with a new septa and degassed under vacuum. The flask was charged with hydrogen and degassed again. This was repeated twice and a hydrogen-filled balloon was attached to the flask. The mixture was stirred at room temperature for 4 hours. Nitrogen was then bubbled through the mixture to displace any excess hydrogen. The mixture was filtered through celite 521 and the filtrate was concentrated under reduced pressure to provide 2-amino-4-methoxybenzoic acid (0.890 g, >99%) as an off-white solid. The crude material was used directly in the next step without characterization.

A mixture of 2-amino-4-methoxybenzoic acid (0.490 g, 3.00 mmol), EDCI (1.12 g, 5.83 mmol), HOBt (0.788 g, 5.83 mmol), N-methylmorpholine (0.590 g, 5.83 mmol) and 14.8 N NH₄OH (0.781 mL, 10.6 mmol) in THF was stirred at room temperature for 16 hours. The solvent was removed under reduced pressure, then the residue was diluted with EtOAc (100 mL), washed with water (2×100 mL), then brine (100 mL), dried over anhydrous Na₂SO₄, filtered, and the solvent was removed under reduced pressure to provide 2-amino-4-methoxybenzamide as a tan solid.

A mixture of 2-amino-4-methoxybenzamide (0.490 g, 3.00 mmol), 4-(2-(tert-butyldimethylsilyloxy)ethoxy)-3,5-dimethylbenzaldehyde (0.925 g, 3.00 mmol), NaHSO₃ (94%, 0.468 g, 4.50 mmol), and p-TsOH.H₂O (0.171 g, 0.900 mmol) in benzene (10.0 mL) was heated at 80° C. for 36 hours. The mixture was cooled to room temperature and the solvent was removed under reduced pressure. The residue was diluted with EtOAc (50 mL), washed with water (50 mL) then brine (50 mL), dried over anhydrous Na₂SO₄, filtered, and the solvent was removed under reduced pressure to provide 2-(4-(2-(tert-butyldimethylsilyloxy)ethoxy)-3,5-dimethylphenyl)-7-methoxyquinazolin-4(3H)-one as a pink solid. The crude material was used directly in the next step without characterization.

A mixture of 2-(4-(2-(tert-butyldimethylsilyloxy)ethoxy)-3,5-dimethylphenyl)-7-methoxyquinazolin-4(3H)-one (1.09 g, 2.30 mmol) in 1 M TBAF (11.6 mL, 11.6 mmol) was stirred at room temperature for 3 hours. The mixture was diluted with water (100 mL) and extracted with EtOAc (2×100 mL). The organic layers were combined, washed with saturated aqueous NH₄Cl (2×75 mL), then brine (100 mL), dried over anhydrous Na₂SO₄, filtered, and the solvent was removed under reduced pressure. The residue was purified over silica gel (12 g, EtOAc/hexanes), triturated in ether, and the product was freeze-dried from MeCN/H₂O to yield the title compound (0.0960 g, 12%) as a white solid. ¹H NMR (300 MHz, DMSO-d₆): δ 12.18 (s, 1H), 8.02 (d, J=8.79 Hz, 1H), 7.91 (s, 2H), 7.16 (d, J=2.46 Hz, 1H), 7.07 (dd, J=8.79, 2.46 Hz, 1H), 4.90 (t, J=5.53 Hz, 1H), 3.91 (s, 3H), 3.89-3.82 (m, 2H), 3.77-3.67 (m, 2H), 2.32 (s, 6H), 2.22 (d, J=6.92 Hz, 1H). MS (APCI) m/z 341 [C₁₉H₂₀N₂O₄+H]⁺.

### Example 29

### Preparation of 5,7-Dimethoxy-2-{3-methyl-4-[2-(5-phenyl-4H-[1,2,4]triazol-3-ylamino)-ethoxy]-phenyl}-3H-quinazolin-4-one

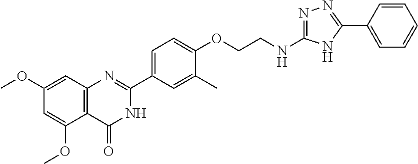 [SMILES: COc1cc(OC)c2c(=O)[nH]c(-c3ccc(OCCNc4nnc(-c5ccccc5)[nH]4)c(C)c3)nc2c1]

To a solution of 2-[4-(2-amino-ethoxy)-3,5-dimethyl-phenyl]-5,7-dimethoxy-3H-quinazolin-4-one (0.37 g, 1.00 mmol) in anhydrous dichloroethane (20 mL) was added benzoyl isothiocyanate (0.18 g 1.10 mmol). The reaction mixture was stirred at room temperature for 3 hours. The solvent was removed and ether (30 mL) was added. The mixture was stirred for 30 minutes and the solid was filtered and dried to give 1-benzoyl-3-{2-[4-(5,7-dimethoxy-4-oxo-3,4-dihydro-quinazolin-2-yl)-2-methyl-phenoxy]-ethyl}-thiourea as a white solid. Yield: 0.53 g (99%).

To a solution of 1-benzoyl-3-{2-[4-(5,7-dimethoxy-4-oxo-3,4-dihydro-quinazolin-2-yl)-2-methyl-phenoxy]-ethyl}-thiourea (0.42 g, 0.785 mmol) in chloroform (20 mL) was added hydrazine hydrate (1.30 mL, 26.5 mmol). The reaction mixture was stirred at reflux for 16 hours. After the solvent was removed, the residue was purified by preparative HPLC to afford the title compound as a white solid. Yield: 35 mg (29%). ¹H NMR (400 MHz, CDCl₃): δ 12.26 (s, 1H), 11.82 (s, 1H), 7.91 (m, 2H), 7.89 (s, 2H), 7.40 (m, 3H), 6.84 (s, 1H), 6.73 (d, J=2.0 Hz, 1H), 6.51 (d, J=2.0 Hz, 1H), 3.98 (t, J=5.6 Hz, 2H), 3.88 (s, 3H), 3.84 (s, 3H), 3.62 (m, 2H), 2.29 (s, 6H). MS (ES⁺) m/z 513.53 (M+1).

### Example 30

### Preparation of 2-{3,5-Dimethyl-4-[2-(3-methyl-[1,2,4]oxadiazol-5-ylamino)-ethoxy]-phenyl}-5,7-dimethoxy-3H-quinazolin-4-one

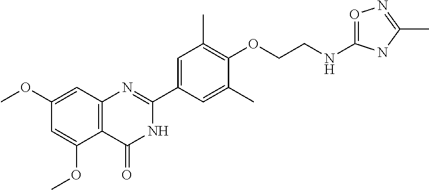

Acetamide oxime (5.00 g, 67.5 mmol) and trichloroacetic anhydride (49.3 mL, 270 mmol) were stirred at 120-130° C. for 3 hours. The mixture was then distilled under vacuum. The fraction at approximately 50-70° C./approximately 5 mmHg was collected. The collected fraction was added to cold saturated aqueous NaHCO₃ and extracted with ethyl acetate. The organic phase was washed with saturated aqueous NaHCO₃ solution and dried over Na₂SO₄. The solvent was evaporated to give 3-methyl-5-trichloromethyl-[1,2,4]oxadiazole as a colorless liquid. Yield: 7.69 g (52%)

A mixture of 3-methyl-5-trichloromethyl-[1,2,4]oxadiazole (56 mg, 0.28 mmol), 2-[4-(2-amino-ethoxy)-3,5-dimethyl-phenyl]-5,7-dimethoxy-3H-quinazolin-4-one (92 mg, 0.25 mmol), and cesium carbonate (179 mg, 0.55 mmol) in DMF (3 mL) was stirred at room temperature under nitrogen for 3.5 days. Water was added, and the mixture was extracted with MeOH/CH₂Cl₂. The organic phase was washed with brine, dried over anhydrous Na₂SO₄, purified by column chromatography (silica gel; 5% MeOH in CH₂Cl₂ as eluent) to give the title compound as a beige solid. Yield: 75 mg (60%).

¹H NMR (400 MHz, CDCl₃): δ 9.68 (s, 1H), 7.71 (s, 2H), 6.82 (d, J=2.4 Hz, 1H), 6.46 (d, J=2.4 Hz, 1H), 5.80 (t, J=5.6 Hz, 1H), 4.00-3.97 (m, 5H), 3.93 (s, 3H), 3.83 (m, 2H), 2.34 (s, 6H), 2.24 (s, 3H). MS (ES⁺) m/z: 452.57 (M+1).

### Example 31

### Preparation of N-{2-[4-(5,7-dimethoxy-4-oxo-3,4-dihydro-pyrido[2,3-d]pyrimidin-2-yl)-2,6-dimethyl-phenoxy]-ethyl}-acetamide

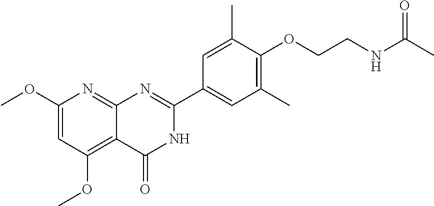 [SMILES: COc1cc(OC)c2c(=O)[nH]c(-c3cc(C)c(OCCNC(C)=O)c(C)c3)nc2n1]

To a solution of 4-hydroxy-3,5-dimethyl-benzaldehyde (15.0 g, 0.10 mol) in anhydrous DMF (30 mL) was added 60% sodium hydride (4.80 g, 0.12 mol) and the reaction mixture was kept stirring for 20 minutes. 2-(2-Bromo-ethyl)isoindole-1,3-dione (25.4 g, 0.10 mol) in anhydrous DMF (30 mL) was added drop-wise. The reaction mixture was heated to 65° C. for 5 hours. Acetic acid (3 mL) was added, DMF was removed, and the residue was poured into water (150 mL), and extracted with dichloromethane (200 mL). The crude compound was purified by column chromatography (silica gel 230-400 mesh; eluting with ethyl acetate and hexane 1:1) to give 4-[2-(1,3-dioxo-1,3-dihydro-isoindol-2-yl)-ethoxy]-3,5-dimethyl-benzaldehyde. Yield: 11.0 g (34%).

To a solution of 2-amino-4,6-dimethoxy-nicotinamide (0.40 g, 2.02 mmol, and 4-[2-(1,3-dioxo-1,3-dihydro-isoindol-2-yl)-ethoxy]-3,5-dimethyl-benzaldehyde (0.65 g, 2.02 mmol) in N,N-dimethylacetamide (30 mL) was added NaHSO₃ (58.5 wt %, 0.40 g, 2.20 mol) and p-TSA (0.12 g, 6.00 mmol). The reaction mixture was heated to 145° C. for 16 hours, and then cooled to room temperature. Solvent was removed under reduced pressure. Aqueous sodium bicarbonate solution (50 mL) was added and the solid separated was filtered and washed with ether (50 mL). Crude compound was purified by column chromatography (silica gel, 230-400 mesh; methanol, ethyl acetate and dichloromethane 5:20:75) to give 2-{2-[4-(5,7-dimethoxy-4-oxo-3,4-dihydro-pyrido[2,3-d]pyrimidin-2-yl)-2,6-dimethyl-phenoxy]-ethyl}-isoindole-1,3-dione as a light yellow solid. Yield: 0.43 g (43%).

Hydrazine hydrate (0.2 mL, 4.1 mmol) was added to a solution of 2-{2-[4-(5,7-dimethoxy-4-oxo-3,4-dihydro-pyrido[2,3-d]pyrimidin-2-yl)-2,6-dimethyl-phenoxy]-ethyl}-isoindole-1,3-dione (0.43 g, 0.86 mmol) in ethanol (10 mL). The reaction mixture was heated to 70° C. for 4 hours, solvent was removed, and the residue was purified by column chromatography (silica gel, 230-400 mesh; eluting with 5% 7 N ammonia in methanol and dichloromethane) to give 2-[4-(2-amino-ethoxy)-3,5-dimethyl-phenyl]-5,7-dimethoxy-3H-pyrido[2,3-d]pyrimidin-4-one as a white solid. Yield: 0.22 g (69%).

To a solution of 2-[4-(2-amino-ethoxy)-3,5-dimethyl-phenyl]-5,7-dimethoxy-3H-pyrido[2,3-d]pyrimidin-4-one (0.21 g, 0.56 mmol) in pyridine (4 mL) and dichloromethane (10 mL) was added acetyl chloride (51 mg, 0.65 mmol), and the reaction mixture was stirred at room temperature for 3 hours. The solvent was removed under reduced pressure, the residue was poured into water (50 mL) and stirred for 30 minutes. The solid separated was filtered and washed with cold water and ether, and then dried under vacuum to give the title compound as a white solid. Yield: 0.19 g (81%). ¹H NMR (400 MHz, DMSO-d₆): δ 8.15 (s, 1H), 7.90 (s, 2H), 6.36 (s, 1H), 3.93 (s, 3H), 3.88 (s, 3H), 3.79 (t, J=5.6 Hz, 3H), 3.42 (q, J=5.6 Hz, 2H), 2.28 (s, 6H), 1.84 (s, 3H). MS (ES) m/z: 411.15 (M−1).

### Example 32

### Preparation of N-(4-(5,7-dimethoxy-4-oxo-3,4-dihydroquinazolin-2-yl)-2,6-dimethylbenzyl)acetamide

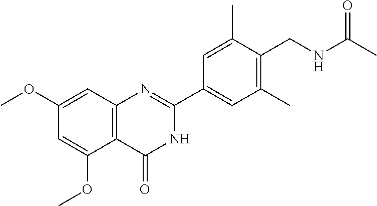 [SMILES: COc1cc(OC)c2c(=O)[nH]c(-c3cc(C)c(CNC(C)=O)c(C)c3)nc2c1]

4-Bromo-2,6-dimethylaniline (4.49 g, 22.4 mmol), water (25 mL) and concentrated HCl (8.0 mL) were sonicated and cooled to 0° C. Sodium nitrite (1.67 g, 24.2 mmol) in water (5 mL) was added over 20 minutes. The mixture was stirred at 0° C. for 30 minutes, and solid Na₂CO₃ was added to adjust the pH to approximately 7. The liquid portion was added, in portions, to copper (I) cyanide (2.42 g, 27.0 mmol) and potassium cyanide (3.65 g, 56.1 mmol) in water (25 mL) at 70° C. over a period of 25 minutes and the mixture was heated at 70° C. for 45 minutes. The mixture was cooled and extracted with toluene (2×150 mL). The organic phase was washed with water (100 mL), then brine (100 mL), dried (Na₂SO₄), filtered, and evaporated to afford a brown oil. Purification by column chromatography (silica gel 230-400 mesh; 25% dichloromethane in hexanes as the eluent) gave 4-bromo-2,6-dimethylbenzonitrile as an orange solid. Yield: 2.3 g (49%).

To 4-bromo-2,6-dimethylbenzonitrile (1.84 g, 8.75 mmol) in anhydrous THF (95 mL), at −78° C. under nitrogen, was added n-butyllithium (2.5 M in hexanes; 3.85 mL, 9.63 mmol) dropwise over 10 minutes. The solution was stirred at −78° C. for 1 hour, and anhydrous DMF (1.00 mL, 12.91 mmol) was added dropwise. The mixture was stirred at −78° C. for 1 hour and at 0° C. for 25 minutes. The reaction was quenched with 1 M HCl, to pH approximately 3. The solution was poured into water (370 mL) and extracted with CHCl₃ (7×100 mL). The organic phase was dried over anhydrous Na₂SO₄, filtered, and evaporated, to give 4-formyl-2,6-dimethylbenzonitrile as a yellow-orange solid (1.20 g, 86%).

4-Formyl-2,6-dimethylbenzonitrile (1.20 g, 7.53 mmol), anhydrous MeOH (80 mL), trimethylorthoformate (18.0 mL, 164.5 mmol), and camphorsulfonic acid (0.050 g, 0.215 mmol) were stirred at room temperature under nitrogen for 23 hours. Triethylamine (7.5 mL) was added and the solution was evaporated to an oil. The oil was diluted with NaHCO₃ (100 mL) and extracted with CHCl₃ (5×75 mL). The organic phase was dried over anhydrous Na₂SO₄, filtered, and evaporated to afford 4-(dimethoxymethyl)-2,6-dimethylbenzonitrile as a golden-red oil. Yield: 1.40 g (90%).

To 4-(dimethoxymethyl)-2,6-dimethylbenzonitrile (0.86 g, 4.18 mmol) in anhydrous THF (40 mL), at 0° C. under nitrogen, was added solid lithium aluminum hydride (0.34 g, 8.94 mmol) in portions over 15 minutes. The mixture was stirred at 0° C. for 30 minutes and at room temperature for 20 hours. The mixture was cooled to 0° C. and quenched with solid Na₂SO₄.10H₂O, stirred for 10 minutes, and then stirred at room temperature for 15 minutes. Solids were removed by filtration and washed with THF (100 mL). The filtrate was evaporated to give (4-(dimethoxymethyl)-2,6-dimethylphenyl)methanamine as a golden-brown semi-solid. Yield: 0.87 g (100%)

To (4-(dimethoxymethyl)-2,6-dimethylphenyl)methanamine (0.87 g, 4.18 mmol), anhydrous CH₂Cl₂ (20 mL), Et₃N (5.84 mL, 41.89 mmol), at 0° C. under nitrogen, was added acetic anhydride (0.44 mL, 4.65 mmol), followed by DMAP (0.018 g, 0.147 mmol). The mixture was stirred at 0° C. for 15 minutes and then at room temperature for 23 hours. The mixture was evaporated to a solid. The solid was stirred with NaHCO₃ (100 mL) and CHCl₃ (50 mL) for 15 minutes. The organic phase was separated and the aqueous phase extracted with CHCl₃ (4×50 mL). The combined organic phase was washed with brine (75 mL), dried over anhydrous Na₂SO₄, filtered, and evaporated to afford N-(4-(dimethoxymethyl)-2,6-dimethylbenzyl)acetamide as a light orange solid (1.00 g, 95%).

To N-(4-(dimethoxymethyl)-2,6-dimethylbenzyl)acetamide (0.83 g, 3.30 mmol) in CHCl₃ (65 mL), at 0° C. was added trifluoroacetic acid/water (1:1, 10 mL) added dropwise. The solution was stirred at 0° C. for 1.75 hours. The solution was diluted with water (200 mL) and the organic phase separated. The aqueous phase was extracted with CHCl₃ (4×75 mL). The combined organic phase was washed with NaHCO₃ (200 mL). The aqueous phase was back-extracted with CHCl₃ (3×30 mL). The combined organic phase was dried (Na₂SO₄), filtered, and evaporated to give a N-(4-formyl-2,6-dimethylbenzyl)acetamide as a brown solid. Yield: 0.56 g (82%)

2-Amino-4,6-dimethoxybenzamide (0.334 g, 1.70 mmol), N-(4-formyl-2,6-dimethylbenzyl)acetamide (0.35 g, 1.70 mmol), anhydrous N,N-dimethylacetamide (10 mL), sodium bisulfite (58.5 wt %, 0.343 g, 1.87 mmol) and p-TsOH.H₂O (0.065 g, 0.341 mmol) were heated at 120° C. for 19.5 hours. The solution was evaporated in vacuo and the residue was triturated with water (50 mL). The yellow solid was filtered off and washed with water (50 mL). The product was purified by column chromatography (silica gel, 230-400 mesh; 6% methanol in dichloromethane as the eluent) and triturated with Et₂O (6 mL) to afford the title compound as a white solid. Yield: 0.202 g (31%). ¹H NMR (400 MHz, DMSO-d₆): δ 11.89 (s, 1H), 7.93 (t, J=4.49 Hz, 1H), 7.85 (s, 2H), 6.74 (d, J=1.95 Hz, 1H), 6.51 (d, J=1.95 Hz, 1H), 4.28 (d, J=4.69 Hz, 2H), 3.87 (s, 3H), 3.83 (s, 3H), 2.37 (s, 6H), 1.80 (s, 3H). MS (ES+) m/z: 382.18 (100%), 383.19.

### Example 33

### Preparation of N-[4-(5,7-dimethoxy-4-oxo-3,4-dihydro-pyrido[2,3-d]pyrimidin-2-yl)-2,6-dimethyl-benzyl]-acetamide

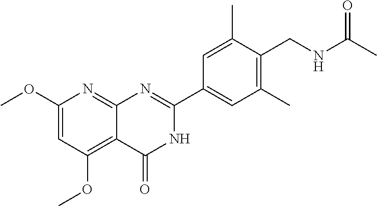 [SMILES: COc1cc(OC)c2c(=O)[nH]c(-c3cc(C)c(CNC(C)=O)c(C)c3)nc2n1]

To a solution of 2-amino-4,6-dimethoxy-nicotinamide (300 mg, 1.52 mmol), N-(4-formyl-2,6-dimethyl-benzyl)-acetamide (342 mg, 1.67 mmol) in N,N-dimethylacetamide (5 mL) were added sodium hydrogen sulfite (58.5 wt %, 300 mg, 1.68 mmol) and p-toluenesulfonic acid monohydrate (60 mg, 0.32 mmol). The reaction mixture was stirred at 150° C. for 17 hours under nitrogen and then cooled to room temperature. The solvent was evaporated under reduced pressure to dryness. Water (50 mL) was added, and extracted with dichloromethane. The organic phase was dried over anhydrous anhydrous sodium sulfate. Solvent was evaporated and the crude compound was purified by column chromatography (silica gel 230-400 mesh; eluting with 5% methanol in dichloromethane) to give the title compound as a white solid. Yield: 78 mg (13%). ¹H NMR (400 MHz, CD₃OD): δ 7.79 (s, 2H), 6.40 (s, 1H), 4.46 (s, 2H), 4.05 (s, 3H), 3.98 (s, 3H), 2.46 (s, 6H), 1.95 (s, 3H). MS (ES⁺) m/z: 383.13 (M+1).

### Example 34

### Preparation of 2-{3,5-dimethyl-4-[2-(2,2,2-trifluoro-ethylamino)-ethoxy]-phenyl}-5,7-dimethoxy-3H-quinazolin-4-one

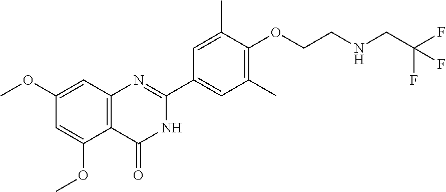 [SMILES: COc1cc(OC)c2c(=O)[nH]c(-c3cc(C)c(OCCNCC(F)(F)F)c(C)c3)nc2c1]

A solution of 2-[4-(2-bromo-ethoxy)-3,5-dimethyl-phenyl]-5,7-dimethoxy-3H-quinazolin-4-one (500 mg, 1.15 mmol) and 2,2,2-trifluoro ethyl amine (1.14 g, 11.53 mmol) and TEA (5 mL) in DMF:THF (10:5 ml) was heated at 40° C. for 24 hours. Then, water (100 mL) was added and product was extracted with ethyl acetate (2×250 mL). The combined organic layer was washed with water, then brine, dried over Na₂SO₄, and evaporated, to give crude product. The crude product was purified by the Simpliflash system, using 2% methanol in dichloromethane as eluent, to give the title compound as a white solid. Yield: 81 mg (15%). ¹H NMR (400 MHz, CDCl₃) δ 9.44 (s, 1H), 7.69 (s, 2H), 6.83 (d, J=2.4 Hz, 1H), 6.46 (d, J=2.4 Hz, 1H), 3.97 (s, 3H), 3.93 (s, 3H), 3.91 (s, br, 2H), 3.33 (d, J=4.4 Hz, 2H), 3.14 (d, J=1.2 Hz, 2H), 2.37 (s, 6H). MS (ES) m/z: 450.07 (M−1) (100%).

### Example 35

### Preparation of N-{2-[4-(6,8-dimethoxy-1-oxo-1,2-dihydro-isoquinolin-3-yl)-2,6-dimethyl-phenoxy]-ethyl}-formamide

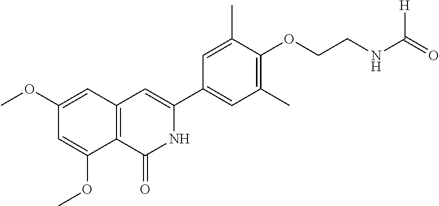 [SMILES: COc1cc(OC)c2c(=O)[nH]c(-c3cc(C)c(OCCNC=O)c(C)c3)cc2c1]

To a suspension of 3-[4-(2-Hydroxy-ethoxy)-3,5-dimethyl-phenyl]-6,8-dimethoxy-2H-isoquinolin-1-one (0.80 g, 2.16 mmol), isoindole-1,3-dione (0.35 g, 2.38 mmol), and triphenyl phosphine (0.85 g, 3.25 mmol) in THF (30 mL), was added diethyl azodicarboxylate (0.56 g, 3.25 mmol), and the reaction mixture was stirred at room temperature for 16 hours. The solvent was evaporated in vacuo and the residue was washed with ether to give 2-{2-[4-(6,8-dimethoxy-1-oxo-1,2-dihydro-isoquinolin-3-yl)-2,6-dimethyl-phenoxy]-ethyl}-isoindole-1,3-dione as an off-white solid. Yield: 1.11 g (crude).

Hydrazine hydrate (0.29 mL, 6.07 mmol) was added to the solution of 2-{2-[4-(6,8-dimethoxy-1-oxo-1,2-dihydro-isoquinolin-3-yl)-2,6-dimethyl-phenoxy]-ethyl}-isoindole-1,3-dione (1.01 g, 2.03 mmol) in ethanol (20 mL). The reaction mixture was heated to 70° C. for 5 hours. The solvent was removed and the residue was purified by the Simpliflash system, using 5% 7 N ammonia in methanol with dichloromethane as eluent, to give 3-[4-(2-amino-ethoxy)-3,5-dimethyl-phenyl]-6,8-dimethoxy-2H-isoquinolin-1-one as a white solid. Yield: 0.59 g (80.2%).

To a solution of 3-[4-(2-amino-ethoxy)-3,5-dimethyl-phenyl]-6,8-dimethoxy-2H-isoquinolin-1-one (0.30 g, 0.8 mmol) in formic acid (20 mL), was heated at reflux for 72 hours. The reaction mixture was cooled to room temperature and solvent was removed under reduced pressure. Water (100 mL) was added to the residue and neutralized with solid NaHCO₃. The product was extracted with dichloromethane (2×200 mL). The combined organic layer was washed with water, then brine, dried over Na₂SO₄, and evaporated to give crude product. The crude product was purified by the Simpliflash system, using 5% 7 N ammonia in methanol with dichloromethane as eluent, to give the title compound as a white solid. Yield: 97 mg (30%). ¹H NMR (400 MHz, DMSO): δ 10.70 (s, 1H), 8.31 (br s, 1H), 8.09 (s, 1H), 7.45 (s, 2H), 6.67 (d, J=2.0 Hz, 1H), 6.64 (s, 1H), 6.45 (d, J=2.0 Hz, 1H), 3.83 (s, 3H), 3.79 (s, 3H), 3.77 (m, 2H), 3.48 (m, 3H), 2.25 (s, 6H). MS (ES) m/z: 397.11 (M+1) (100%).

### Example 36

### Preparation of 2-(3,5-dimethyl-4-(2-(methylamino)ethoxy)phenyl)-5,7-dimethoxyquinazolin-4(3H)-one

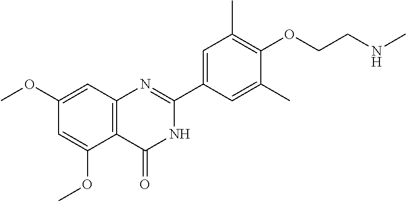

To a mixture of 2-(4-(2-hydroxyethoxy)-3,5-dimethylphenyl)-5,7-dimethoxyquinazolin-4(3H)-one (2.00 g, 5.40 mmol) and Et₃N (0.977 mL, 7.02 mmol) in CH₂Cl₂ (27.0 mL) was added slowly MsCl (0.543 mL, 7.02 mmol) at room temperature. After 1 day, additional Et₃N (0.977 mL, 7.02 mmol) and MsCl (0.543 mL, 7.02 mmol) was added and the mixture was stirred for 2 hours, then diluted with EtOAc (300 mL) and washed with 10% aqueous citric acid (3×75 mL), saturated aqueous NaHCO₃ (75 mL), and brine (75 mL). An insoluble white solid was collected by filtration to provide 2-(4-(5,7-dimethoxy-4-oxo-3,4-dihydroquinazolin-2-yl)-2,6-dimethyl-phenoxy)ethyl methanesulfonate (0.890 g, 37%).

A mixture of compound 2-(4-(5,7-dimethoxy-4-oxo-3,4-dihydroquinazolin-2-yl)-2,6-dimethyl-phenoxy)ethyl methanesulfonate (0.200 g, 0.446 mmol) and 33% CH₃NH₂ in EtOH (5.00 mL) was heated at reflux overnight. The solvent was removed under vacuum and the residue was purified on silica gel (12 g, CH₂Cl₂/CH₃OH) and the product freeze-dried from MeCN/H₂O to provide the title compound (0.0968 g, 57%) as a light yellow solid. ¹H NMR (300 MHz, DMSO-d₆: δ 7.90 (s, 2H), 6.73 (d, J=2.29 Hz, 1H), 6.52 (d, J=2.29 Hz, 1H), 3.94-3.80 (m, 8H), 2.98 (t, J=5.46 Hz, 2H), 2.45 (s, 3H), 2.33-2.28 (m, 8H). MS (APCI) m/z 384 [C₂₁H₂₆N₃O₄+H]⁺.

### Example 37

### Preparation of N-(2-(4-(5,7-dimethoxy-4-oxo-3,4-dihydroquinazolin-2-yl)-2,6-dimethylphenoxy)ethyl)propane-2-sulfonamide

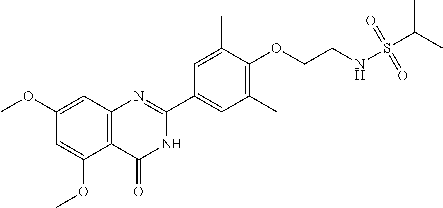 [SMILES: COc1cc(OC)c2c(=O)[nH]c(-c3cc(C)c(OCCNS(=O)(=O)C(C)C)c(C)c3)nc2c1]

A mixture of 3,5-dimethyl-4-hydroxybenzaldehyde (0.600 g, 4.00 mmol), N-(2-bromoethyl)-phthalimide (1.22 g, 4.80 mmol), K₂CO₃ (0.829 g, 6.00 mmol), NaI (3.00 g, 20.0 mmol) in DMF (40.0 mL) was heated at 80° C. for 2.5 hours. The reaction was cooled to room temperature, diluted with EtOAc (200 mL), washed with 1 M NaOH (2×100 mL), 1 M HCl (2×100 mL), brine (75 mL), dried over sodium sulfate, filtered, and concentrated under vacuum. The residue was chromatographed on silica gel (40 g, hexanes/EtOAc) to provide the expected ether (0.300 g, 23%) as a yellow solid. A mixture of this ether (0.293 g, 0.907 mmol), 2-amino-4,6-dimethoxybenzamide (0.178 g, 0.907 mmol), NaHSO₃ (94%, 0.100 g, 0.907 mmol), and p-TsOH.H₂O (0.0173 g, 0.0907 mmol) in DMA (11.3 mL) was stirred at reflux for 1.5 hours, then cooled to room temperature. The mixture was diluted with EtOAc (250 mL), washed with saturated aqueous ammonium chloride (3×75 mL), them brine (75 mL), dried over sodium sulfate, filtered, and concentrated under vacuum. The residue was chromatographed on silica gel (40 g, CH₂Cl₂/CH₃OH) to provide the expected product (0.075 g, 17%) as a light yellow solid. A mixture of the above compound (0.213 g, 0.426 mmol) and 2 M methylamine in THF (25.0 mL) was stirred at room temperature for 17 hours. The volatiles were removed under vacuum and 2-(4-(2-aminoethoxy)-3,5-dimethylphenyl)-5,7-dimethoxyquinazolin-4(3H)-one was isolated (0.036 g, 23%) as a white solid.

A mixture of 2-(4-(2-aminoethoxy)-3,5-dimethylphenyl)-5,7-dimethoxyquinazolin-4(3H)-one (0.125 g, 0.338 mmol), 2-propylsulfonyl chloride (0.040 mL, 0.36 mmol), and DBU (0.100 mL, 0.67 mmol) in THF (2.5 mL) was stirred at 60° C. for 18 hours. Then, the mixture was cooled to room temperature and purified by silica gel chromatography, eluting with 92:7:1 CHCl₃/MeOH/concentrated NH₄OH. The mixture was further purified by reverse-phase HPLC, eluting with 10% to 90% CH₃CN in H₂O with 0.1% TFA, to afford the desired product. The product was freeze-dried from CH₃CN/H₂O to afford the title compound (0.080 g, 50%) as a white solid. ¹H NMR (300 MHz, DMSO-d₆: δ 11.85 (s, 1H), 8.09 (s, 2H), 7.33 (t, J=6.0 Hz, 1H), 6.74 (d, J=2.3 Hz, 1H), 6.52 (d, J=2.3 Hz, 1H), 3.89 (s, 3H), 3.82-3.86 (m, 5H), 3.21-3.39 (m, 3H), 2.31 (s, 6H), 1.26 (d, J=6.8 Hz, 6H). APCI MS m/z 476 [M+H]⁺.

### Example 38

### Preparation of 2-(4-(2-(isopropylamino)ethoxy)-3,5-dimethylphenyl)-5,7-dimethoxyquinazolin-4(3H)-one

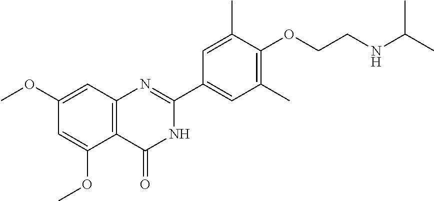 [SMILES: COc1cc(OC)c2c(=O)[nH]c(-c3cc(C)c(OCCNC(C)C)c(C)c3)nc2c1]

A solution of 2-(4-(2-aminoethoxy)-3,5-dimethylphenyl)-5,7-dimethoxyquinazolin-4(3H)-one (0.200 g, 0.54 mmol) in EtOH (10 mL) and acetone (0.198 mL, 2.71 mmol) was treated with PtO₂ (0.050 g). The reaction mixture was stirred under 1 atmosphere of hydrogen for 48 hours. Then, the mixture was filtered through celite with ethanol washings, concentrated, and purified by silica gel chromatography, to afford the title compound (0.155 g, 70%). The product was further purified by reverse-phase HPLC, eluting with 10% to 90% CH₃CN in H₂O with 0.1% TFA, to afford the title compound as a white solid. ¹H NMR (300 MHz, DMSO-d₆: δ 7.90 (s, 2H), 6.74 (d, J=2.3 Hz, 1H), 6.52 (s, J=2.3 Hz, 1H), 3.83-3.89 (m, 8H), 2.89 (t, J=5.6 Hz, 2H), 2.75-2.84 (m, 1H), 2.30 (s, 6H), 1.01 (d, J=6.2 Hz, 6H); APCI MS m/z 412 [M+H]⁺.

### Example 39

### Preparation of N-(2-(4-(5,7-dimethoxy-4-oxo-3,4-dihydroquinazolin-2-yl)-2-methylphenoxy)ethyl)acetamide

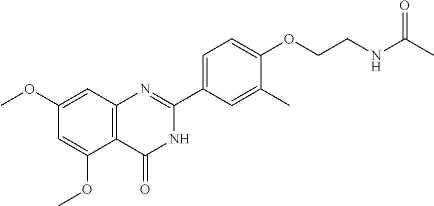 [SMILES: COc1cc(OC)c2c(=O)[nH]c(-c3ccc(OCCNC(C)=O)c(C)c3)nc2c1]

2-(4-(2-Aminoethoxy)-3-methylphenyl)-5,7-dimethoxyquinazolin-4(3H)-one was synthesized as described for 2-(4-(2-aminoethoxy)-3,5-dimethylphenyl)-5,7-dimethoxyquinazolin-4(3H)-one from 3-methyl-4-hydroxybenzaldehyde (See Example 43).

A suspension of 2-(4-(2-aminoethoxy)-3-methylphenyl)-5,7-dimethoxyquinazolin-4(3H)-one (0.12 g, 0.33 mmol) in CH₂Cl₂ (5 mL) was treated with Et₃N (0.05 mL, 0.41 mmol) and acetyl chloride (0.026 mL, 0.37 mmol) and the mixture stirred at room temperature for 3 hours. Then, the mixture was concentrated in vacuo and the residue purified by flash chromatography on silica gel, eluting with 97:3 to 90:10 CH₂Cl₂/MeOH to 92:7:1 CHCl₃/MeOH/concentrated NH₄OH, to afford crude product. Further purification on a reverse-phase O₁₈ column, eluting with 10% to 90% CH₃CN in H₂O with 0.05% TFA, afforded the title compound (0.080 g, 61%) as a white solid. ¹H NMR (300 MHz, DMSO-d₆) δ 11.65 (s, 1H), 7.93-8.18 (m, 3H), 7.05 (d, J=8.4 Hz, 1H), 6.71 (d, J=2.3 Hz, 1H), 6.50 (d, J=2.3 Hz, 1H), 4.07 (t, J=5.6 Hz, 2H), 3.88 (s, 3H), 3.84 (s, 3H), 3.35-3.52 (m, 2H), 2.23 (s, 3H), 1.83 (s, 3H). APCI MS m/z 398 [M+H]⁺.

### Example 40

### Preparation of 2-(4-(2-(dimethylamino)ethoxy)-3,5-dimethylphenyl)-5,7-dimethoxyquinazolin-4(3H)-one

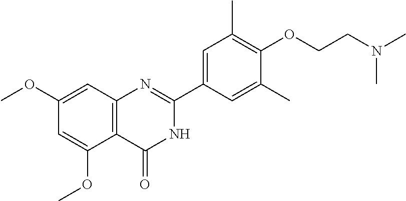

To a solution of 2-(4-(2-aminoethoxy)-3,5-dimethylphenyl)-5,7-dimethoxyquinazolin-4(3H)-one (0.150 g, 0.41 mmol) in MeOH (16 mL) and CH₂Cl₂ (5 mL) was added 37% aqueous formaldehyde (0.300 mL, 4.0 mmol) and the mixture stirred for 1 hour. Then, NaBH₄ (0.078 g, 2.05 mmol) was added and the reaction was stirred for 16 hours at room temperature. Additional 37% aqueous formaldehyde (1.0 mL) was added and stirred for 1 hour, at which time, additional NaBH₄ (0.100 g, 2.63 mmol) was added and stirred for 1 hour. The reaction mixture was concentrated, redissolved in CH₂Cl₂, washed with brine (100 mL), dried (Na₂SO₄), filtered, and concentrated. The residue was purified by silica gel chromatography, eluting with 9:1 CH₂Cl₂/MeOH to 92:7:1 CHCl₃/MeOH/concentrated aqueous NH₄OH. The residue was further purified by reverse-phase HPLC, eluting with 10% to 90% CH₃CN in H₂O with 0.1% TFA, to afford the title compound as a white solid (0.070 g, 43%). ¹H NMR (300 MHz, DMSO-d₆) δ 11.70 (br s, 1H), 7.90 (s, 2H), 6.74 (d, J=2.3 Hz, 1H), 6.52 (d, J=2.3 Hz, 1H), 3.84-3.89 (m, 8H), 2.64 (t, J=5.8 Hz, 2H), 2.30 (s, 6H), 2.24 (s, 6H). APCI MS m/z 398 [M+H]⁺.

### Example 41

### Preparation of N-(2-(4-(5,7-dimethoxy-4-oxo-3,4-dihydroquinazolin-2-yl)-2,6-dimethylphenoxy)ethyl)-N-methylacetamide

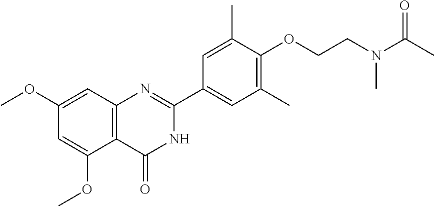 [SMILES: COc1cc(OC)c2c(=O)[nH]c(-c3cc(C)c(OCCN(C)C(C)=O)c(C)c3)nc2c1]

To a solution of 2-(3,5-dimethyl-4-(2-(methylamino)ethoxy)phenyl)-5,7-dimethoxyquinazolin-4(3H)-one (0.110 g, 0.287 mmol) in CH₂Cl₂ (10 mL) was added Et₃N (0.080 mL, 0.574 mmol), followed by acetyl chloride (0.022 mL, 0.315 mmol). The mixture was stirred at room temperature under nitrogen for 10 minutes, concentrated, and purified by silica gel chromatography, eluting with 9:1 CH₂Cl₂/MeOH, followed by reverse-phase HPLC, eluting with 10% to 90% CH₃CN in H₂O with 0.1% TFA, to afford the title compound as a white solid (0.078 g, 64%). ¹H NMR (mixture of amide rotomers, 300 MHz, DMSO-d₆: δ 11.85 (s, 1H), 7.90 (d, J=2.7 Hz, 2H), 6.74 (d, J=2.2 Hz, 1H), 6.52 (d, J=2.2 Hz, 1H), 3.84-3.95 (m, 8H), 3.65-3.74 (m, 2H), 3.12 (s, 1.5H), 2.92 (s, 1.5H), 2.27 (d, J=1.1 Hz, 6H), 2.11 (s, 1.5H), 2.03 (s, 1.5H). APCI MS m/z 424 [M−H]⁻.

### Example 42

### Preparation of N-(2-(4-(5,7-dimethoxy-4-oxo-3,4-dihydroquinazolin-2-yl) 2,6-dimethylphenoxy)ethyl)formamide

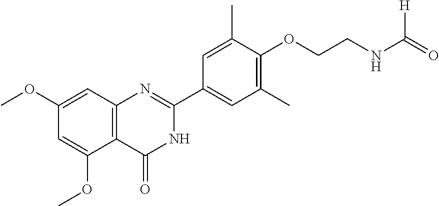 [SMILES: COc1cc(OC)c2c(=O)[nH]c(-c3cc(C)c(OCCNC=O)c(C)c3)nc2c1]

A solution of 2-(4-(2-aminoethoxy)-3,5-dimethylphenyl)-5,7-dimethoxyquinazolin-4(3H)-one (0.086 g, 0.23 mmol) in ethanol (10 mL) and methyl formate (0.028 mL, 0.46 mmol) was stirred at room temperature for 5 hours. At this time, an additional portion of methyl formate (5 mL, 80.6 mmol) was added and the mixture heated at reflux for 4 days. The mixture was concentrated and purified by silica gel chromatography, eluting with 92:7:1 CHCl₃/MeOH/concentrated NH₄OH. The product was freeze-dried from CH₃CN/H₂O to yield the title compound (0.065 g, 71%) as a white solid. ¹H NMR (300 MHz, DMSO-d₆: δ 11.84 (s, 1H), 8.29-8.37 (m, 1H), 8.11 (d, J=1.3 Hz, 1H), 7.90 (s, 2H), 6.74 (d, J=2.3 Hz, 1H), 6.52 (d, J=2.3 Hz, 1H), 3.89 (s, 3H), 3.79-3.84 (m, 5H), 3.47-3.53 (m, 2H), 2.29 (s, 6H). APCI MS m/z 396 [M−H]⁻.

### Example 43

### Preparation of N-(2-(4-(5,7-dimethoxy-4-oxo-3,4-dihydroquinazolin-2-yl) 2,6-dimethylphenoxy)ethyl)-N-methylformamide

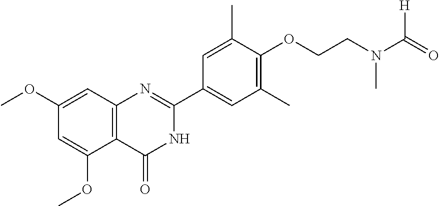 [SMILES: COc1cc(OC)c2c(=O)[nH]c(-c3cc(C)c(OCCN(C)C=O)c(C)c3)nc2c1]

To a solution of 2-(3,5-dimethyl-4-(2-(methylamino)ethoxy)phenyl)-5,7-dimethoxyquinazolin-4(3H)-one (0.080 g, 0.21 mmol) in EtOH (15 mL) was added methyl formate (5 mL). The mixture was heated at reflux for 24 hours, concentrated, and purified by silica gel chromatography, eluting with 9:1 CH₂Cl₂/MeOH, to afford the title compound as a white solid (0.080 g, 93%): ¹H NMR (mixture of amide rotomers, 300 MHz, DMSO-d₆: δ 11.85 (s, 1H), 8.12 (d, J=1.9 Hz, 1H), 7.90 (s, 2H), 6.74 (d, J=2.2 Hz, 1H), 6.52 (d, J=2.2 Hz, 1H), 3.88-3.93 (m, 5H), 3.84 (s, 3H), 3.62-3.68 (m, 2H), 3.08 (s, 0.5H), 2.88 (s, 0.5H), 2.25-2.35 (m, 6H); APCI MS m/z 410 [M−H]⁻.

### Example 44

### Preparation of N-(2-(4-(5,7-dimethoxy-4-oxo-3,4-dihydroquinazolin-2-yl)-2,6-dimethylphenoxy)ethyl)dimethylamino-N-sulfonamide

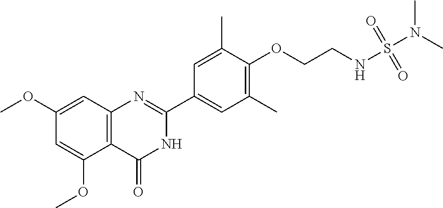

A solution of 2-(4-(2-aminoethoxy)-3,5-dimethylphenyl)-5,7-dimethoxyquinazolin-4(3H)-one (0.150 g, 0.41 mmol) in CH₂Cl₂(10 mL) was treated with Et₃N (0.083 g, 0.82 mmol), then dimethylsulfamoylchloride (0.065 g, 0.45 mmol), and the reaction mixture stirred under nitrogen at room temperature for 1 hour. Then, DBU (0.100 mL) was added and stirring continued for 1 hour at room temperature. Then, the reaction mixture was heated at reflux for 18 hours, additional dimethylsulfamoylchloride (0.150 mL) was added, and heating continued at reflux for a further 2 hours. The reaction mixture was cooled and purified by flash chromatography on silica gel, eluting with 100% CH₂Cl₂ to 100% (92:7:1 CHCl₃/MeOH/concentrated NH₄OH). The resulting solid was further purified by reverse-phase HPLC, eluting with 10% to 90% CH₃CN in H₂O with 0.1% TFA. The solids were then triturated with CH₃CN to afford the title compound as a white solid. ¹H NMR (300 MHz, CDCl₃) δ 9.20 (s, 1H), 7.69 (s, 2H), 6.82 (d, J=2.3 Hz, 1H), 6.5 (d, J=2.3 Hz, 1H), 4.72-4.80 (m, 1H), 3.93-3.98 (m, 8H), 3.46-3.56 (m, 2H), 2.87 (s, 6H), 2.38 (s, 6H); ESI MS m/z 477 [M+H]⁺.

### Example 45

### Preparation of N-(2-(4-(5,7-dimethoxy-4-oxo-3,4-dihydroquinazolin-2-yl) 2,6-dimethylphenoxy)ethyl)cyanamide

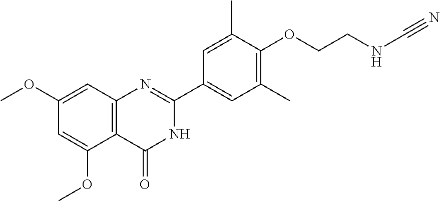 [SMILES: COc1cc(OC)c2c(=O)[nH]c(-c3cc(C)c(OCCNC#N)c(C)c3)nc2c1]

To a solution of 2-(4-(2-aminoethoxy)-3,5-dimethylphenyl)-5,7-dimethoxyquinazolin-4(3H)-one (0.150 g, 0.41 mmol) in MeOH (15 mL) was added BrCN (0.043 g, 0.41 mmol) and NaHCO₃ (0.044 g, 0.52 mmol). The reaction was stirred at room temperature for 1 hour and then concentrated in vacuo. Purification by flash chromatography on silica gel, eluting with 2% to 10% MeOH/CH₂Cl₂, afforded the title compound (0.120 g, 74%) as a white solid. ¹H NMR (300 MHz, DMSO-d₆): δ 11.85 (s, 1H), 7.82-7.92 (m, 2H), 7.03-7.14 (m, 1H), 6.72 (d, J=1.4 Hz, 1H), 6.59 (d, J=1.4 Hz, 1H), 3.81-3.93 (m, 8H), 3.15-3.29 (m, 2H), 2.28 (s, 6H). APCI MS m/z 395 [M+H]⁺.

### Example 46

### Preparation of 2-(3,5-dimethyl-4-(2-(5-methylisoxazol-3-ylamino)ethoxy)phenyl)-5,7-dimethoxyquinazolin-4(3H)-one

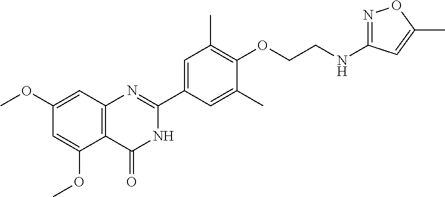

To a solution of 5-methylisoxazol-3-amine (1.0 g, 10.2 mmol) in CH₂Cl₂ was added Et₃N (1.03 g, 10.2 mmol) and bromoacetyl chloride (1.60 g, 10.2 mmol). The mixture was stirred at room temperature for 1 hour, washed with water (100 mL), then brine (100 mL), dried (Na₂SO₄), filtered, and concentrated, to afford 2-bromo-N-(5-methylisoxazol-3-yl)acetamide as a white solid (1.2 g, 55%).

To a solution of 2-bromo-N-(5-methylisoxazol-3-yl)acetamide (0.223 g, 1.0 mmol) in THF (10 mL) under nitrogen was added 1.0 M BH₃.THF (3.0 mL, 3.0 mmol). The reaction mixture was stirred at room temperature for 18 hours, quenched with 1 M NaOH, extracted with ethyl acetate (2×100 mL), dried over anhydrous Na₂SO₄, filtered, and concentrated. The residue was purified by flash chromatography on silica gel, eluting with 1:1 ethyl acetate/hexane to 100% ethyl acetate, to afford N-(2-bromoethyl)-5-methylisoxazol-3-amine as a white solid (0.061 g, 30%).

To a solution of 4-hydroxy-3,5-dimethylbenzaldehyde (0.036 g, 0.24 mmol) in DMF (1.5 mL) was added K₂CO₃ (0.050 g, 0.36 mmol) and the mixture stirred at room temperature under nitrogen for 30 minutes. After this time, a solution of N-(2-bromoethyl)-5-methylisoxazol-3-amine (0.060 g, 0.29 mmol) in DMF (1.5 mL) was added and the reaction heated at reflux for 2 hours. The mixture was concentrated and purified by flash chromatography on silica gel, eluting with 1:1 ethyl acetate/heptane to 100% ethyl acetate, to afford 3,5-dimethyl-4-(2-(5-methylisoxazol-3-ylamino)ethoxy)benzaldehyde (0.028 g, 26%).

A mixture of 3,5-dimethyl-4-(2-(5-methylisoxazol-3-ylamino)ethoxy)benzaldehyde (0.121 g, 0.44 mmol), 2-amino-4,6-dimethoxybenzamide (0.087 g, 0.44 mmol), NaHSO₃ (0.050 g, 0.48 mmol), and p-TsOH (0.008 g, 0.044 mmol) in DMA (3 mL) was heated at 155° C. under nitrogen for 9 hours. Then, the reaction mixture was cooled, diluted with ethyl acetate (200 mL), and washed with water (100 mL), brine (100 mL), dried over anhydrous Na₂SO₄, filtered, and concentrated. The residue was purified by flash chromatography on silica gel, eluting with 100% CH₂Cl₂ to 100% 92:7:1 CHCl₃/MeOH/concentrated NH₄OH, to afford the title compound (0.129 g, 65%). ¹H NMR (300 MHz, DMSO-d₆: δ 11.99 (s, 1H), 7.99 (s, 2H), 6.77 (d, J=2.3 Hz, 1H), 6.55 (d, J=2.3 Hz, 1H), 5.29 (s, 1H), 4.70-4.72 (m, 1H), 3.90 (s, 3H), 3.85 (s, 3H), 3.55-3.61 (m, 4H), 2.22 (s, 6H), 2.21 (s, 3H). APCI MS m/z 451 [M+H]⁺.

### Example 47

### Preparation of 2-(3,5-dimethyl-4-(2-(pyrimidin-2-ylamino)ethoxy)phenyl)-5,7-dimethoxyquinazolin-4(3H)-one

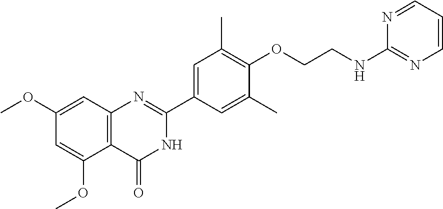 [SMILES: COc1cc(OC)c2c(=O)[nH]c(-c3cc(C)c(OCCNc4ncccn4)c(C)c3)nc2c1]

To a solution of 2-(4-(2-aminoethoxy)-3,5-dimethylphenyl)-5,7-dimethoxyquinazolin-4(3H)-one (0.145 g, 0.40 mmol) in t-butanol (10 mL) was added Et₃N (0.06 mL, 0.47 mmol) and 2-chloropyrimidine (0.045 g, 0.40 mmol). The reaction was stirred and heated at reflux temperature overnight, then concentrated in vacuo. Purification by flash chromatography on silica gel, eluting with 95:5 CH₂Cl₂/MeOH, afforded the title compound (0.038 g, 21%) as a white solid. ¹H NMR (300 MHz, DMSO-d₆): δ 8.29 (d, J=4.7 Hz, 2H), 7.87 (s, 2H), 7.31 (t, J=6.1 Hz, 1H), 6.72 (d, J=2.3 Hz, 1H), 6.58 (t, J=4.7 Hz, 1H), 6.51 (s, 1H), 3.95 (t, J=5.9 Hz, 1H), 3.88 (s, 3H), 3.84 (s, 3H), 3.65-3.71 (m, 2H), 2.25 (s, 6H). ESI MS m/z 448 [M+H]⁺.

### Example 48

### Preparation of 2-(4-(2-(isoxazol-3-ylamino)ethoxy)-3,5-dimethylphenyl)-5,7-dimethoxyquinazolin-4(3H)-one

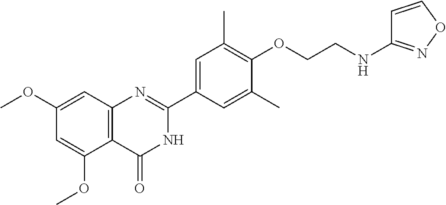 [SMILES: COc1cc(OC)c2c(=O)[nH]c(-c3cc(C)c(OCCNc4ccon4)c(C)c3)nc2c1]

To a solution of isoxazol-3-amine (2.28 g, 27.1 mmol) in CH₂Cl₂ at 0° C. under nitrogen was added Et₃N (2.74 g, 27.1 mmol), followed by bromoacetyl chloride (4.26 g, 27.1 mmol). The mixture was warmed to room temperature, stirred for 2 hours, washed sequentially with water (200 mL) and brine (200 mL), dried (Na₂SO₄), filtered, and concentrated, to afford 2-bromo-N-(isoxazol-3-yl)acetamide as a tan solid (4.5 g, 81%).

To a solution of 2-bromo-N-(isoxazol-3-yl)acetamide (1.0 g, 4.9 mmol) in THF (50 mL) under nitrogen was added 1.0 M BH₃.THF (14.6 mL, 14.6 mmol). The mixture was stirred at room temperature for 3.5 hours and then an additional portion of BH₃.THF (5.0 mL, 5.0 mmol) was added. After an additional 15 hours at room temperature, the reaction was quenched with 1 M NaOH, extracted with ethyl acetate (2×150 mL), dried (Na₂SO₄), filtered, and concentrated. The residue was purified by flash chromatography on silica gel, eluting with 1:1 ethyl acetate/heptane to 100% ethyl acetate, to afford N-(2-bromoethyl)isoxazol-3-amine (0.133 g, 14%).

To a solution of 4-hydroxy-3,5-dimethylbenzaldehyde (0.471 g, 3.14 mmol) in DMF (20 mL) was added K₂CO₃ (0.650 g, 4.71 mmol). The reaction mixture was stirred at room temperature under nitrogen for 30 minutes. Then, a solution of N-(2-bromoethyl)isoxazol-3-amine (0.600 g, 3.14 mmol) in DMF (10 mL) was added. The mixture was heated at reflux for 3 hours, concentrated, and purified by flash chromatography on silica gel, eluting with 30% ethyl acetate/heptane to 100% ethyl acetate, to afford 4-(2-(isoxazol-3-ylamino)ethoxy)-3,5-dimethylbenzaldehyde as a white solid (0.260 g, 32%).

A mixture of 4-(2-(isoxazol-3-ylamino)ethoxy)-3,5-dimethylbenzaldehyde (0.253 g, 0.97 mmol), 2-amino-4,6-dimethoxybenzamide (0.190 g, 0.97 mmol), NaHSO₃ (0.111 g, 1.07 mmol), and p-TsOH (0.018 g, 0.097 mmol) in DMA (10 mL) was heated at 150° C. under nitrogen for 44 hours. Then, the reaction mixture was concentrated, diluted with ethyl acetate (200 mL), and washed with water (150 mL), then brine (150 mL), dried over anhydrous Na₂SO₄, filtered, and concentrated. The residue was purified by flash chromatography on silica gel, eluting with 100% CH₂Cl₂ to 100% 92:7:1 CHCl₃/MeOH/concentrated NH₄OH, to afford the title compound (0.150 g, 35%). ¹H NMR (300 MHz, DMSO-d₆: δ 11.82 (s, 1H), 8.39 (d, J=1.7 Hz, 1H), 7.89 (s, 2H), 6.73 (d, J=2.2 Hz, 1H), 6.51 (d, J=2.2 Hz, 1H), 6.44 (t, J=6.1 Hz, 1H), 6.02 (d, J=1.7 Hz, 1H), 3.94 (t, J=5.5 Hz, 2H), 3.89 (s, 3H), 3.84 (s, 3H), 3.46-3.51 (m, 2H), 2.27 (s, 6H). APCI MS m/z 437 [M+H]⁺.

### Example 49

### Preparation of 2-(4-(2-(4,6-dimethoxypyrimidin-2-ylamino)ethoxy)-3,5-dimethylphenyl)-5,7-dimethoxyquinazolin-4(3H)-one

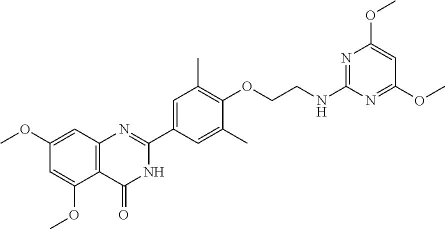

Following the method described for Example 51 above, the title compound was made from 2-chloro-4,6-dimethoxypyrimidine (0.071 g, 0.40 mmol) in 35% yield. ¹H NMR (300 MHz, DMSO-d₆): δ 11.82 (s, 1H), 7.88 (s, 2H), 7.22 (t, J=6.1 Hz, 1H), 6.72 (d, J=2.3 Hz, 1H), 6.51 (s, 1H), 5.38 (s, 1H), 3.90-4.02 (m, 2H), 3.88 (s, 3H), 3.84 (s, 3H), 3.77 (s, 6H), 3.59-3.72 (m, 2H), 2.27 (s, 6H). APCI MS m/z 506 [M−H]⁻.

### Example 50

### Preparation of 2-[4-(3-hydroxy-propyl)-3,5-dimethoxyphenyl]-5,7-dimethoxy-3H-quinazolin-4-one

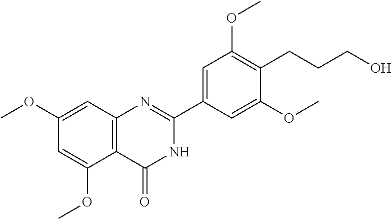 [SMILES: COc1cc(OC)c2c(=O)[nH]c(-c3cc(OC)c(CCCO)c(OC)c3)nc2c1]

To a stirred solution of 4-hydroxy-3,5-dimethoxylbenzaldehyde (5.87 g, 32.2 mmol) in CH₂Cl₂ (50 mL) and pyridine (8.6 mL) was added trifluoromethanesulfonic anhydride (10.0 g, 35.4 mmol) at 0° C. After the addition was complete, stirring was continued for 16 hours at room temperature. The reaction mixture was diluted with ethyl acetate (150 mL) and washed with water (3×100 mL). The separated organic phase was dried over anhydrous sodium sulfate, filtered, and concentrated. The crude product, trifluoromethanesulfonic acid 4-formyl-2,6-dimethoxyphenyl ester, was used in the next step without further purification. Yield: 10.0 g (98.9%).

To a stirred solution of trifluoromethanesulfonic acid 4-formyl-2,6-dimethoxyphenyl ester (8.00 g, 25.4 mmol) in anhydrous DMF (80 mL) under nitrogen at room temperature were sequentially added triethylamine (5.14 g, 50.8 mmol), methyl acrylate (21.9 g, 254.0 mmol), 1,3-bis-(diphenylphosphino)-propane (0.84 g, 2.03 mmol), and palladium acetate (0.40 g, 1.77 mmol). The reaction mixture was stirred at 115° C. for 16 hours. DMF was removed under reduced pressure and the residue was taken in ethyl acetate (200 mL) and washed with 1 N HCl solution (2×50 mL), and saturated sodium bicarbonate solution (100 mL). The organic phase was dried over anhydrous sodium sulfate, filtered, and concentrated. The residue was purified by column chromatography (silica gel 230-400 mesh; eluting with hexane/ethyl acetate=3:1) to give 3-(4-formyl-2,6-dimethoxyphenyl)-acrylic acid methyl ester. Yield: 4.0 g (62%).

To a solution of 3-(4-formyl-2,6-dimethoxyphenyl)-acrylic acid methyl ester (5.00 g, 20.0 mmol) in methanol (80 mL), 1.5 N sodium hydroxide (45 mL) was added. The suspension was stirred at room temperature for 16 hours. Methanol was evaporated and acetic acid (4.0 mL) was added. The aqueous layer was extracted with dichloromethane (200 mL) then acidified, to pH 3, with 2 N HCl. The solid was filtered and further washed with cold water (100 mL) to obtain 3-(4-formyl-2,6-dimethoxyphenyl)-acrylic acid as a yellow solid. Yield: 4.20 g (89%).

To a solution of 3-(4-formyl-2,6-dimethoxyphenyl)-acrylic acid (4.20 g, 17.7 mmol) and N,N-diisopropylethylamine (3.5 mL) in ethanol (80 mL) were added Pd/C (400 mg, 10 wt %). The suspension was vigorously stirred under 1 bar of hydrogen pressure for 16 hours. The mixture was filtered through a celite pad and the filtrate was evaporated. The residue was poured into chilled 1 N HCl (200 mL), the solid was filtered, and further washed with cold water (100 mL) to give a mixture of 3-(4-formyl-2,6-dimethoxyphenyl)-propionic acid and 3-(4-hydroxymethyl-2,6-dimethoxyphenyl)-propionic acid as a white solid. Yield: 3.30 g.

To a suspension of LiAlH₄ (1.00 g, 26.3 mmol) in anhydrous THF (40 mL) was added dropwise a solution of a mixture of 3-(4-formyl-2,6-dimethoxyphenyl)-propionic acid and 3-(4-hydroxymethyl-2,6-dimethoxyphenyl)-propionic acid (3.30 g, 13.8 mmol). After the addition was complete, the reaction mixture was stirred at reflux for 2 hours. The suspension was diluted with THF (20 mL) and another portion of LiAlH₄ (0.60 g, 15.8 mmol) was added. The mixture was refluxed for an additional 1 hour. The reaction was cooled to room temperature, carefully quenched with aqueous saturated NH₄Cl solution (8 mL), acidified to pH 1-2 with 2 N HCl, and extracted with ethyl acetate (200 mL). The organic phase was dried over sodium sulfate, filtered and concentrated to provide 3-(4-hydroxymethyl-2,6-dimethoxyphenyl)-propan-1-ol as a colorless crystalline solid. Yield: 3.08 g (98.7%).

To a solution of 3-(4-hydroxymethyl-2,6-dimethoxyphenyl)-propan-1-ol (3.08 g, 13.6 mmol) in ethanol (50 mL) was added activated MnO₂ (4.15 g, 47.6 mmol) and the resulting suspension was stirred at reflux for 16 hours. The reaction mixture was filtered through a celite pad and the filtrate was concentrated. The residue was purified by column chromatography (silica gel 230-400 mesh; eluting with 2:1 hexane and ethyl acetate) to give 4-(3-hydroxy-propyl)-3,5-dimethoxybenzaldehyde. Yield: 1.10 g (36%).

To a solution of 2-amino-4,6-dimethoxy-benzamide (0.35 g, 1.78 mmol) and 4-(3-hydroxy-propyl)-3,5-dimethylbenzaldehyde (0.40 g, 1.78 mmol) in N,N-dimethylacetamide (8 mL) were added NaHSO₃ (0.35 g, 1.96 mmol) and p-TSA (34 mg, 0.18 mmol) and the reaction mixture was heated at 115-120° C. for 5 hours, then cooled to room temperature. N,N-dimethylacetamide was removed under reduced pressure. The residue was diluted with water (50 mL) and the pH was adjusted to 7 by adding sodium bicarbonate solution. The solid was collected and washed with ether and further mixed with methanol (30 mL) and stirred for 1 hour, filtered, and dried under vacuum to give the title compound as a white solid. Yield: 0.25 g (35%). ¹H NMR (400 MHz, CDCl₃): δ 11.13 (s, 1H), 7.30 (s, 2H), 6.86 (d, J=2.4 Hz, 1H), 6.47 (d, J=2.4 Hz, 1H), 3.98 (s, 6H), 3.95 (s, 3H), 3.94 (s, 3H), 3.52 (m, 2H), 2.86 (t, J=6.6 Hz 2H), 2.27 (t, J=6.6 Hz, 1H), 1.81 (m, 2H). MS (ES⁺) m/z: 401.49 (M+1).

### Example 51

### Preparation of 2-[4-(3-hydroxy-propyl)-3-methoxy-phenyl]-5,7-dimethoxy-3H-quinazolin-4-one

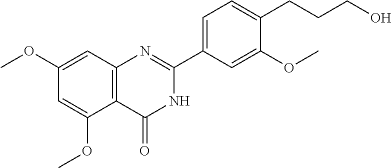 [SMILES: COc1cc(OC)c2c(=O)[nH]c(-c3ccc(CCCO)c(OC)c3)nc2c1]

To a stirred solution of 4-hydroxy-3-methoxy-benzaldehyde (5.00 g, 32.8 mmol) in CH₂Cl₂ (50 mL) and pyridine (8 mL) was added trifluoromethanesulfonic anhydride (10.19 g, 36.1 mmol) at 0° C. After addition was complete, stirring was continued for 16 hours at room temperature. The reaction mixture was diluted with ethyl acetate (200 mL) and washed with water (3×100 mL) and brine (100 mL). The separated organic layer was dried over anhydrous sodium sulfate, filtered, and concentrated. The crude product was purified by column chromatography (silica gel 230-400 mesh; 20% ethyl acetate in hexanes as eluent) to give trifluoromethanesulfonic acid 4-formyl-2-methoxy-phenyl ester. Yield: 8.00 g, (85%).

To a stirred solution of trifluoromethanesulfonic acid 4-formyl-2-methoxy-phenyl ester (5.00 g, 17.5 mmol) in anhydrous DMF (75 mL) under nitrogen at room temperature were sequentially added triethylamine (3.50 g, 34.5 mmol), ethyl acrylate (17.50 g, 174.7 mmol), 1,3-bis-(diphenylphosphino)-propane (0.40 g, 0.96 mmol), and palladium (II) acetate (0.20 g, 0.87 mmol). The reaction mixture was stirred at 100° C. for 5 hours. DMF was removed under reduced pressure, and the residue was taken in ethyl acetate (200 mL) and washed with 1 N HCl solution (2×50 mL), and saturated sodium bicarbonate solution (100 mL) and brine (100 mL). The organic phase was dried over sodium sulfate, filtered, and concentrated. The residue was purified by column chromatography (silica gel 230-400 mesh; 20% ethyl acetate in hexanes as eluent) to give 3-(4-formyl-2-methoxy-phenyl)-acrylic acid ethyl ester as a beige solid. Yield: 3.00 g (73%).

To a solution of 3-(4-formyl-2-methoxy-phenyl)-acrylic acid ethyl ester (3.00 g, 13.6 mmol) and N,N-diisopropylethylamine (3.0 mL) in ethanol (100 mL) were added Pd/C (10 wt %, 400 mg). The suspension was hydrogenated under 25 psi pressure for 5 hours. The mixture was filtered through a celite pad and the filtrate was evaporated. The residue was poured into chilled 1 N HCl (200 mL), the solid was filtered, and further washed with cold water (100 mL) to give a 3-(4-hydroxymethyl-2-methoxy-phenyl)-propionic acid ethyl ester as a beige solid. Yield: 2.80 g (93%).

To a suspension of LiAlH₄ (0.51 g, 26.3 mmol) in anhydrous THF (100 mL) was added dropwise a solution of 3-(4-hydroxymethyl-2-methoxy-phenyl)-propionic acid ethyl ester (2.5 g, 11.1 mmol) in THF (10 mL). After the addition was complete, the reaction mixture was stirred at reflux for 3 hours. Then, the reaction was cooled to room temperature, carefully quenched with aqueous saturated NH₄Cl solution (8 mL), acidified to pH approximately 1-2 with 2 N HCl, and extracted with ethyl acetate (200 mL). The organic phase was dried over sodium sulfate, filtered, and concentrated, to provide 3-(4-hydroxymethyl-2-methoxy-phenyl)-propan-1-ol as a colorless crystalline solid. Yield: 1.80 g (90%).

To a solution of 3-(4-hydroxymethyl-2-methoxy-phenyl)-propan-1-ol (1.8 g, 9.1 mmol) in ethanol (50 mL) was added activated MnO₂ (2.79 g, 32.0 mmol) and the resulting suspension was stirred at reflux for 16 hours. The reaction mixture was filtered through celite pad and the filtrate was concentrated. The residue was purified by column chromatography (silica gel 230-400 mesh; 2:1 hexane and ethyl acetate as eluent) to give 4-(3-hydroxy-propyl)-3-methoxy-benzaldehyde. Yield: 1.2 g (67%).

To a solution of 2-amino-4,6-dimethoxy-benzamide (0.48 g, 2.44 mmol) and 4-(3-hydroxy-propyl)-3-methoxy-benzaldehyde (0.40 g, 2.05 mmol) in N,N-dimethylacetamide (10 mL) were added NaHSO₃ (58.5 wt %, 0.40 g, 2.25 mmol) and p-toluenesulfonic acid monohydrate (78 mg, 0.41 mmol) and the reaction mixture was heated at 115° C. for 16 hours, then cooled to room temperature. The solvent was removed under reduced pressure. The residue was diluted with water (50 mL) and the pH was adjusted to approximately 7 by adding sodium bicarbonate solution. The solid was filtered and washed with water. The crude compound was purified by column chromatography (silica gel 230-400 mesh; 5% methanol in dichloromethane as eluent) to give the title compound as an off-white solid. Yield: 0.35 g (46%). ¹H NMR (400 MHz, DMSO-d₆): δ 12.02(s, 1H), 7.75-7.73 (m, 2H), 7.28 (d, J=7.8 Hz, 1H), 6.75 (d, J=2.3 Hz, 1H), 6.53 (d, J=1.9 Hz, 1H), 4.48 (t, J=5.0 Hz, 1H), 3.90 (d, J=4.2 Hz, 6H), 3.85 (s, 3H), 3.44 (q, J=6.6 Hz, 2H), 2.65 (t, J=7.4 Hz 2H), 1.71-1.67 (m, 2H). MS (ES⁺) m/z: 371.51 (M+1).

### Example 52

### Preparation of 2-[2-(2-hydroxyethyl)-1H-indol-6-yl]-5,7-dimethoxy-3H-quinazolin-4-one

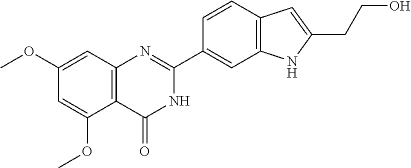 [SMILES: COc1cc(OC)c2c(=O)[nH]c(-c3ccc4cc(CCO)[nH]c4c3)nc2c1]

To a degassed solution of methyl-3-amino-4-iodobenzoate (2.00 g, 7.22 mmol) in a mixture of 5:1 DMF-triethylamine (30 mL) were added PdCl₂(PPh₃)₂ (0.25 g, 0.36 mmol) and copper (I) iodide (0.41 g, 2.16 mmol) and the mixture was degassed again. A degassed solution of 2-(3-butynyloxy)tetrahydro-2H-pyran (1.7 mL, 10.83 mmol) in a mixture of 5:1 DMF-triethylamine (12 mL) was added drop-wise at 75° C. over a period of 45 minutes under nitrogen. Soon after the addition, TLC showed completion of the reaction. The reaction mixture was cooled to room temperature, solvent was removed under reduced pressure, and the residue was diluted with water (75 mL) and extracted with ethyl acetate (3×50 mL). The organic phase was washed with water (50 mL), brine (50 mL), and dried over anhydrous MgSO₄. The solvent was evaporated and the crude product was purified by column chromatography (silica gel 230-400 mesh; 2:1 hexanes and ethyl acetate as eluent) to obtain 3-amino-4-[4-(tetrahydropyran-2-yloxy)-but-1-ynyl]benzoic acid methyl ester as a brown solid. Yield: 1.70 g (78%).

To a stirred solution of 3-amino-4-[4-(tetrahydropyran-2-yloxy)-but-1-ynyl]benzoic acid methyl ester (1.68 g, 5.55 mmol) in anhydrous pyridine (5 mL) was added acetyl chloride (0.43 mL, 6.11 mmol) at 0° C. under nitrogen. Stirring was continued at 0° C. After 30 minutes TLC showed completion of the reaction. Pyridine was removed under reduced pressure and the residue was diluted with ethyl acetate (100 mL). The resulting mixture was washed with aq 2 N HCl (20 mL), water (2×15 mL) and brine (20 mL). After drying over anhydrous MgSO₄, solvent was removed to obtain 3-acetylamino-4-[4-(tetrahydropyran-2-yloxy)-but-1-ynyl]benzoic acid methyl ester as a beige solid. Yield: 1.67 g (87%). Crude product was used in the next step without further purification.

A 1.0 M solution of tetrabutylammonium fluoride (9.67 mL, 9.67 mmol) in THF was added to a solution of 3-acetylamino-4-[4-(tetrahydropyran-2-yloxy)-but-1-ynyl]benzoic acid methyl ester (1.67 g, 4.83 mmol) in anhydrous THF (20 mL) at room temperature. The resulting reddish-brown solution was heated at reflux for 2 hours and then allowed to cool to room temperature. The solvent was removed under reduced pressure and the residue was taken in water (50 mL) and extracted with ethyl acetate (3×50 mL). The organic phase was washed with water (25 mL), brine (50 mL), and dried over anhydrous MgSO₄. The solvent was evaporated and the crude product was purified by column chromatography on (silica gel 230-400 mesh; dichloromethane as eluent) to give 2-[2-(tetrahydropyran-2-yloxy)ethyl]-1H-indole-6-carboxylic acid methyl ester as a light brown solid. Yield: 1.27 g (87%).

To a suspension of lithium aluminum hydride (0.32 g, 8.37 mmol) in anhydrous THF (20 mL) was added a solution of 2-[2-(tetrahydropyran-2-yloxy)ethyl]-1H-indole-6-carboxylic acid methyl ester (1.27 g, 4.19 mmol) in anhydrous THF (10 mL) at −30° C. to −20° C. dropwise over a period of 15 minutes under nitrogen. The temperature was allowed to warm to room temperature and stirring continued for 15 hours. The reaction mixture was quenched with saturated aqueous ammonium chloride solution at 0° C., diluted with ethyl acetate (50 mL), and filtered. The solid was washed with ethyl acetate. The combined organic phase was dried over anhydrous MgSO₄. The solvent was evaporated and the crude product was purified by the Simpliflash system (3:2 ethyl acetate-hexanes as eluent) to give {2-[2-(tetrahydropyran-2-yloxy)ethyl]-1H-indol-6-yl}-methanol as a white solid. Yield: 0.61 g (53%).

IBX (0.62 g, 2.21 mmol) was added to a solution of {2-[2-(tetrahydropyran-2-yloxy)ethyl]-1H-indol-6-yl}-methanol (0.61 g, 2.21 mmol) in DMSO (10 mL). After 30 min, the reaction mixture became a clear solution. Stirring was continued at room temperature for 2 hours and during this time, some solid precipitated. Water (50 mL) was added, the solid was filtered, and washed with ethyl acetate (50 mL). The filtrate was collected and extracted with ethyl acetate (3×20 mL). The organic phase was washed with brine (30 mL) and dried over anhydrous MgSO₄. Removal of solvent gave 2-[2-(tetrahydropyran-2-yloxy)ethyl]-1H-indole-6-carbaldehyde as a light brown solid. Yield: 0.60 g (99%).

To a solution of 2-amino-4,6-dimethoxy-benzamide (0.48 g, 2.42 mmol) and 2-[2-(tetrahydropyran-2-yloxy)ethyl]-1H-indole-6-carbaldehyde (0.60 g, 2.20 mmol) in N,N-dimethylacetamide (20 mL) were added NaHSO₃ (58.5 wt %, 0.60 g, 3.30 mmol) and p-toluenesulfonic acid monohydrate (0.17 g, 0.88 mmol). The reaction mixture was heated at 110° C. for 20 hours and then cooled to room temperature. N,N-dimethylacetamide was removed under reduced pressure. The residue was diluted with saturated sodium carbonate solution (50 mL) and extracted with dichloromethane (4×25 mL). The combined organic phase was washed with brine and dried over anhydrous magnesium sulfate. The solvent was removed and the crude product was purified by column chromatography (silica gel 230-400 mesh; 7% methanol in dichloromethane as eluent). Yield: 0.45 g (56%). The compound was further purified by preparative HPLC to give the title compound as an off-white solid. Yield: 123 mg. ¹H NMR (400 MHz, DMSO-d₆): δ 11.89 (s, 1H), 11.25 (s, 1H), 8.18 (s, 1H), 7.82 (d, J=8.40 Hz, 1H), 7.50 (d, J=8.40 Hz, 1H), 6.73 (d, J=2.4 Hz, 1H), 6.49 (d, J=2.0 Hz, 1H), 6.27 (s, 1H), 4.80 (t, J=5.2 Hz, 1H), 3.90 (s, 3H), 3.85 (s, 3H), 3.78-3.73 (m, 2H), 2.92 (t, J=7.2 Hz, 2H). MS (ES+) m/z 366.54 (100%, M+1).

### Example 53

### Inhibition of Tetra-Acetylated Histone H4 Binding Individual BET Bromodomains

Proteins were cloned and overexpressed with a N-terminal 6×His tag, then purified by nickel affinity followed by size exclusion chromatography. Briefly, E. coli BL21(DE3) cells were transformed with a recombinant expression vector encoding N-terminally Nickel affinity tagged bromodomains from Brd2, Brd3, Brd4. Cell cultures were incubated at 37° C. with shaking to the appropriate density and induced overnight with IPTG. The supernatant of lysed cells was loaded onto Ni-IDA column for purification. Eluted protein was pooled, concentrated and further purified by size exclusion chromatography. Fractions representing monomeric protein are pooled, concentrated, aliquoted, and frozen at −80° C. for use in subsequent experiments.

Binding of tetra-acetylated histone H4 and BET bromodomains was confirmed by a Time Resolved Fluorescence Resonance Energy Transfer (TR-FRET) method. N-terminally His-tagged bromodomains (200 nM) and biotinylated tetra-acetylated histone H4 peptide (25-50 nM, Millipore) were incubated in the presence of Europium Cryptate-labeled streptavidin (Cisbio Cat. #610SAKLB) and XL665-labeled monoclonal anti-His antibody (Cisbio Cat. #61 HISXLB) in a white 96 well microtiter plate (Greiner). For inhibition assays, serially diluted test compound was added to these reactions in a 0.2% final concentration of DMSO. Final buffer concentrations were 30 mM HEPES pH 7.4, 30 mM NaCl, 0.3 mM CHAPS, 20 mM phosphate pH 7.0, 320 mM KF, 0.08% BSA). After 2 hours incubation at room temperature, the fluorescence by FRET was measured at 665 and 620 nm by a SynergyH4 plate reader (Biotek). Illustrative results with the first bromodomain of Brd4 Results are shown in Table 2. The binding inhibitory activity was shown by a decrease in 665 nm fluorescence relative to 620 nm. IC₅₀ values were determined from a dose response curve. Compounds with an IC₅₀ value less than 50 uM were deemed to be active.

TABLE 2
Inhibition of Binding of Tetra-acetylated Histone
H4 and Brd4 bromodomain 1 as Measured by FRET

| | FRET |
|---|---|
| | activity |
| Compound | (<50 uM) |
| 7-(2-amino-ethoxy)-2-(4-hydroxy-3,5-dimethyl-phenyl)-5- | Active |
| methoxy-3H-quinazolin-4-one (Example 3) | |
| 2-(4-hydroxy-3,5-dimethyl-phenyl)-5-methoxy-7-(2- | Active |
| methoxy-ethoxy)-3H-quinazolin-4-one (Example 4) | |
| 2-(4-hydroxy-3,5-dimethylphenyl)-5-methoxy-7-[2- | Active |
| (pyridin-3-ylmethoxy)ethoxy]-3H-quinazolin-4-one | |
| (Example 6) | |
| 7-(2-dimethylamino-ethoxy)-2-(4-hydroxy-3,5- | Active |
| dimethylphenyl)-3H-quinazolin-4-one (Example 7) | |
| 2-(4-hydroxy-3,5-dimethyl-phenyl)-6-(pyridin-4-ylamino)- | Active |
| 3H-quinazolin-4-one (Example 8) | |
| 2-(4-hydroxy-3,5-dimethyl-phenyl)-6-(pyridin-2-ylamino)- | Inactive |
| 3H-quinazolin-4-one (Example 9) | |
| 2-(4-hydroxy-3,5-dimethylphenyl)-6-((4-methylpiperazin- | Active |
| 1-yl)methyl)quinazolin-4(3H)-one (Example 10) | |
| N-((2-(4-hydroxy-3,5-dimethylphenyl)-4-oxo-3,4- | Active |
| dihydroquinazolin-6-yl)methyl)methanesulfonamide | |
| (Example 11) | |
| 2-(4-(2-(benzyloxy)ethoxy)-3,5-dimethylphenyl)-5,7- | Inactive |
| dimethoxypyrido[2,3-d]pyrimidin-4(3H)-one (Example 12) | |
| 2-(4-(2-hydroxyethoxy)-3,5-dimethylphenyl)-5,7- | Active |
| dimethylpyrido[2,3-d]pyrimidin-4(3H)-one (Example 13) | |
| 5,7-difluoro-2-(4-(2-hydroxyethoxy)-3,5- | Inactive |
| dimethylphenyl)quinazolin-4(3H)-one (Example 14) | |
| 2-[4-(2,3-Dihydroxy-propoxy)-3,5-dimethyl-phenyl]-5,7- | Active |
| dimethoxy-3H-quinazolin-4-one (Example 16) | |
| 2-[4-(2-hydroxy-ethoxy)-phenyl]-5,7-dimethoxy-3H- | Active |
| quinazolin-4-one (Example 17) | |
| 2-[4-(2-hydroxy-ethoxy)-naphthalen-1-yl]-5,7-dimethoxy- | Active |
| 3H-quinazolin-4-one (Example 18) | |
| 2-(2-hydroxymethyl-benzofuran-5-yl)-5,7-dimethoxy-3H- | Inactive |
| quinazolin-4-one (Example 19) | |
| 7-(2-benzyloxy-ethoxy)-2-[4-(2-hydroxy-ethoxy)-3,5- | Active |
| dimethyl-phenyl]-5-methoxy-3H-quinazolin-4-one | |
| (Example 20) | |
| 7-(2-benzyloxy-ethoxy)-2-(2-hydroxymethyl-benzofuran- | Inactive |
| 5-yl)-5-methoxy-3H-quinazolin-4-one (Example 21) | |
| 2-[4-(5,7-dimethoxy-4-oxo-3,4-dihydro-quinazolin-2-yl)- | Active |
| 2,6-dimethyl-phenoxy]-N-methyl-acetamide (Example | |
| 22) | |
| 2-[4-(5,7-Dimethoxy-4-oxo-3,4-dihydro-quinazolin-2-yl)- | Active |
| 2,6-dimethyl-phenoxy]-N-(4-methoxy-phenyl)-acetamide | |
| (Example 23) | |
| N-benzyl-2-[4-(5,7-dimethoxy-4-oxo-3,4- | Active |
| dihydroquinazolin-2-yl)-2,6-dimethylphenoxy]acetamide | |
| (Example 24) | |
| 2-[4-(4-hydroxy-butoxy)-3,5-dimethyl-phenyl]-5,7- | Active |
| dimethoxy-3H-quinazolin-4-one (Example 25) | |
| 7-chloro-2-(4-(2-hydroxyethoxy)-3,5- | Inactive |
| dimethylphenyl)quinazolin-4(3H)-one (Example 26) | |
| 5-chloro-2-(4-(2-hydroxyethoxy)-3,5- | Inactive |
| dimethylphenyl)quinazolin-4(3H)-one (Example 27) | |
| 2-(4-(2-hydroxyethoxy)-3,5-dimethylphenyl)-7- | Inactive |
| methoxyquinazolin-4(3H)-one (Example 28) | |
| 5,7-Dimethoxy-2-{3-methyl-4-[2-(5-phenyl-4H- | Active |
| [1,2,4]triazol-3-ylamino)-ethoxy]-phenyl}-3H-quinazolin-4- | |
| one (Example 29) | |
| 2-{3,5-Dimethyl-4-[2-(3-methyl-[1,2,4]oxadiazol-5- | Active |
| ylamino)-ethoxy]-phenyl}-5,7-dimethoxy-3H-quinazolin-4- | |
| one (Example 30) | |
| N-{2-[4-(5,7-dimethoxy-4-oxo-3,4-dihydro-pyrido[2,3- | Active |
| d]pyrimidin-2-yl)-2,6-dimethyl-phenoxy]-ethyl}-acetamide | |
| (Example 31) | |
| N-(4-(5,7-dimethoxy-4-oxo-3,4-dihydroquinazolin-2-yl)- | Active |
| 2,6-dimethylbenzyl)acetamide (Example 32) | |
| N-[4-(5,7-dimethoxy-4-oxo-3,4-dihydro-pyrido[2,3- | Active |
| d]pyrimidin-2-yl)-2,6-dimethyl-benzyl]-acetamide | |
| (Example 33) | |
| 2-{3,5-dimethyl-4-[2-(2, 2, 2-trifluoro-ethylamino)-ethoxy]- | Inactive |
| phenyl}-5,7-dimethoxy-3H-quinazolin-4-one (Example | |
| 34) | |
| N-{2-[4-(6,8-dimethoxy-1-oxo-1,2-dihydro-isoquinolin-3- | Active |
| yl)-2,6-dimethyl-phenoxy]-ethyl}-formamide (Example 35) | |
| 2-(3,5-dimethyl-4-(2-(methylamino)ethoxy)phenyl)-5,7- | Active |
| dimethoxyquinazolin-4(3H)-one (Example 36) | |
| N-(2-(4-(5,7-dimethoxy-4-oxo-3,4-dihydroquinazolin-2- | Active |
| yl)-2,6-dimethylphenoxy)ethyl)propane-2-sulfonamide | |
| (Example 37) | |
| 2-(4-(2-(isopropylamino)ethoxy)-3,5-dimethylphenyl)-5,7- | Active |
| dimethoxyquinazolin-4(3H)-one (Example 38) | |
| N-(2-(4-(5,7-dimethoxy-4-oxo-3,4-dihydroquinazolin-2- | Active |
| yl)-2-methylphenoxy)ethyl)acetamide (Example 39) | |
| 2-(4-(2-(dimethylamino)ethoxy)-3,5-dimethylphenyl)-5,7- | Active |
| dimethoxyquinazolin-4(3H)-one (Example 40) | |
| N-(2-(4-(5,7-dimethoxy-4-oxo-3,4-dihydroquinazolin-2- | Active |
| yl)-2,6-dimethylphenoxy)ethyl)-N-methylacetamide | |
| (Example 41) | |
| N-(2-(4-(5,7-dimethoxy-4-oxo-3,4-dihydroquinazolin-2- | Active |
| yl)-2,6-dimethylphenoxy)ethyl)formamide (Example 42) | |
| N-(2-(4-(5,7-dimethoxy-4-oxo-3,4-dihydroquinazolin-2- | Active |
| yl)-2,6-dimethylphenoxy)ethyl)-N-methylformamide | |
| (Example 43) | |
| N-(2-(4-(5,7-dimethoxy-4-oxo-3,4-dihydroquinazolin-2- | Active |
| yl)-2,6-dimethylphenoxy)ethyl)dimethylamino-N- | |
| sulfonamide (Example 44) | |
| N-(2-(4-(5,7-dimethoxy-4-oxo-3,4-dihydroquinazolin-2- | Active |
| yl)-2,6-dimethylphenoxy)ethyl)cyanamide (Example 45) | |
| 2-(3,5-dimethyl-4-(2-(5-methylisoxazol-3- | Active |
| ylamino)ethoxy)phenyl)-5,7-dimethoxyquinazolin-4(3H)- | |
| one (Example 46) | |
| 2-(3,5-dimethyl-4-(2-(pyrimidin-2- | Inactive |
| ylamino)ethoxy)phenyl)-5,7-dimethoxyquinazolin-4(3H)- | |
| one (Example 47) | |
| 2-(4-(2-(isoxazol-3-ylamino)ethoxy)-3,5-dimethylphenyl)- | Active |
| 5,7-dimethoxyquinazolin-4(3H)-one (Example 48) | |
| 2-(4-(2-(4,6-dimethoxypyrimidin-2-ylamino)ethoxy)-3,5- | Inactive |
| dimethylphenyl)-5,7-dimethoxyquinazolin-4(3H)-one | |
| (Example 49) | |
| 2-[4-(3-hydroxy-propyl)-3,5-dimethoxyphenyl]-5,7- | Active |
| dimethoxy-3H-quinazolin-4-one (Example 50) | |
| 2-[4-(3-hydroxy-propyl)-3-methoxy-phenyl]-5,7- | Active |
| dimethoxy-3H-quinazolin-4-one (Example 51) | |
| 2-[2-(2-hydroxyethyl)-1H-indol-6-yl]-5,7-dimethoxy-3H- | Active |
| quinazolin-4-one (Example 52) | |

### Example 54

### Inhibition of c-Myc Expression in Cancer Cell Lines

MV4-11 cells (2.5×10⁴ cells) were plated in 96 well U-bottom plates with test compound or DMSO (0.1%), and incubated for 3 hours at 37° C. Cells were then harvested by centrifugation, lysed, and mRNA was isolated using the mRNA catcher plus kit (Invitrogen). Reverse transcription of the mRNA and duplex amplification of the c-myc and cyclophilin cDNAs was performed using the RNA Ultrasense kit (Invitrogen) and a ViiA7 real-time PCR machine (Applied Biosystems). IC₅₀ values were determined from a dose response curve. Compounds with an IC₅₀ value less than 30 uM were deemed to be active.

TABLE 3
Inhibition of c-myc Activity in Human AML MV4-11 cells

| | c-myc activity |
|---|---|
| Compound | (<30 uM) |
| N-(4-(5,7-dimethoxy-4-oxo-3,4-dihydroquinazolin-2- | Inactive |
| yl)-2,6-dimethylbenzyl)acetamide (Example 32) | |
| 2-(4-(2-(isopropylamino)ethoxy)-3,5-dimethylphenyl)- | Active |
| 5,7-dimethoxyquinazolin-4(3H)-one (Example 38) | |

### Example 55

### Inhibition of Cell Proliferation in Human AML MV-4-11 Cells

MV4-11 cells: 96-well plates were seeded with 5×10⁴ cells per well of exponentially growing human AML MV-4-11 (CRL-9591) cells and immediately treated with two-fold dilutions of test compounds, ranging from 30 μM to 0.2 μM. Triplicate wells were used for each concentration, as well as a media only and three DMSO control wells. The cells and compounds were incubated at 37° C., 5% CO₂ for 72 hours before adding 20 μL of the CellTiter Aqueous One Solution (Promega) to each well and incubating at 37° C., 5% CO₂ for an additional 3-4 hours. The absorbance was taken at 490 nm in a spectrophotometer and the percentage of proliferation relative to DMSO-treated cells was calculated after correction from the blank well. IC₅₀ were calculated using the GraphPad Prism software. Compounds with an IC₅₀ value less than 30 uM were deemed to be active.

TABLE 4
Inhibition of Cell Proliferation in Human AML MV-4-11 cells

| | cell proliferation |
|---|---|
| Compound | activity (<30 uM) |
| N-(4-(5,7-dimethoxy-4-oxo-3,4-dihydroquinazolin- | Inactive |
| 2-yl)-2,6-dimethylbenzyl)acetamide (Example 32) | |
| 2-(4-(2-(isopropylamino)ethoxy)-3,5-dimethylphenyl)- | Active |
| 5,7-dimethoxyquinazolin-4(3H)-one (Example 38) | |

### Example 56

### Lipopolysaccharide (LPS) Stimulated Whole Blood Assay for Measuring TNFa and IL-6 Levels

Activation of monocytic cells by agonists of toll-like receptors such as bacterial lipopolysaccharide (LPS) results in production of key inflammatory mediators including IL-6 and TNFa. Such pathways are widely considered to be central to the pathophysiology of a range of auto-immune and inflammatory disorders. Compounds to be tested are diluted to give a range of appropriate concentrations and 1 μl of the dilution stocks is added to wells of a 96 plate. Following addition of whole blood (130 μL) the plates are incubated at 37 degrees (5% CO₂) for 30 min before the addition of 10 μl of 2.8 μg/mL lipopolysaccharides (LPS), diluted in complete RPMI 1640 (final concentration=200 ng/mL), to give a total volume of 140 μL per well. After further incubation for 24 hours at 37 degrees, 140 μL of PBS are added to each well. The plates are sealed, shaken for 10 minutes and then centrifuged (2500 rpm×10 min). 100 uL of the supernatant are removed and IL-6 and TNFa levels assayed by immunoassay (typically by MesoScale Discovery technology) either immediately or following storage at −20 degrees. BET inhibitors tested in this assay will inhibit the production of the key inflammatory mediator IL-6 and/or TNFa.

### Example 57

### In Vivo Mouse Endotoxemia Model Assay

High doses of Endotoxin (bacterial lipopolysaccharide) are administered to animals produce a profound shock syndrome including a strong inflammatory response, dysregulation of cardiovascular function, organ failure and ultimately mortality. This pattern of response is very similar to human sepsis and septic shock, where the body's response to a significant bacterial infection can be similarly life threatening. To test the compounds for use in the invention groups of Balb/c male mice are given a lethal dose of 15 mg/kg LPS by intraperitoneal injection. Ninety minutes later, animals are dosed intravenously with vehicle (20% cyclodextrin 1% ethanol in apyrogen water) or test compound (10 mg/kg). The survival of animals is evaluated at 4 days. BET inhibitors tested in the mouse endotoxemia model assay will result in a significant animal survival effect following intravenous administration.

### Example 58

### Growth Suppressive Activity Test Against Cancer Cells

Using RPMI 1640 medium (manufactured by SIGMA) supplemented with 10% fetal bovine serum, human promyelocytic leukemia-derived cell line HL-60, human acute lymphoblastic leukemia-derived cell line MOLT4, human Burkitt's lymphoma-derived cell line Daudi, and human multiple myeloma-derived cell line RPMI-8226 are each cultured at 37° C., 5% CO₂. In addition, using ISKOV medium (manufactured by SIGMA) supplemented with 10% fetal bovine serum, human chronic myeloid leukemia-derived cell line MV4-11 is cultured at 37° C., 5% CO₂. Moreover, using DMEM/F-12 medium (manufactured by SIGMA) supplemented with 10% fetal bovine serum, human lung cancer cell-derived cell line EBC-1, human hepatocellular cancer-derived cell line Kim-1, human colorectal cancer-derived cell line HCT-116, human prostate cancer-derived cell line PC-3, human ovarian cancer-derived cell line A2780, and human osteosarcoma-derived cell line Saos2 are each cultured at 37° C., 5% CO₂. These cells are plated on a 96 well plate, and cultured for 1 day. To each culture test compound diluted with the medium to a final concentration of 0.0003⁻¹⁰ μm (final DMSO concentration, 0.4%) is added. After culture for 3 more days, WST-8 (0.16 mg/mL) is added to the culture medium and the cells are cultured for 2 hr. The absorbance at 650 nm is subtracted from the absorbance at 450 nm. The growth suppressive activity is shown by a decrease rate of the absorbance of the group receiving test compound to that of the control group, and GI₅₀ value is determined from a dose-reaction curve plotting a decrease rate of the absorbance obtained by changing the compound compound concentrations.

This assay demonstrates that a compound that inhibits binding between acetylated histone, more specifically acetylated histone H4, and a bromodomain-containing protein, more specifically human-derived BET family protein BRD2, BRD3 or BRD4 can be used as an antitumor agent.

### Example 59

### HIV Tat-Mediated Transactivation Inhibition Assay

This assay evaluates inhibition of Tat-mediated transactivation by BET inhibitors that block the PCAF bromodomain interaction with HIV-1 Tat-AcK50. The effect is assessed by a microinjection study as described previously by Dorr et al. (EMBO J. 21; 2715-2723, 2002). In this microinjection assay, HeLa-Tat cells are grown on Cellocate coverslips and microinjected at room temperature with an automated injection system (Carl Zeiss). Samples are prepared as a 20 μl injection mix containing the LTR-luciferase (100 ng/ml) and CMV-GFP (50 ng/ml) constructs together with 5 mg/ml a chemical compound or pre-immune IgGs. Live cells are examined on a Zeiss Axiovert microscope to determine the number of GFP-positive cells. Four hours after injection, cells are washed in cold phosphate buffer and processed for luciferase assays (Promega). BET inhibitors tested in this assay will inhibit Tat-mediated transactivation by the PCAF BRD inhibitor.

What is claimed is:

1. A method of inhibiting BET (bromodomain and extra terminal domain protein) proteins comprising administering to the subject in need thereof, a therapeutically effective amount of at least one compound of Formula I: 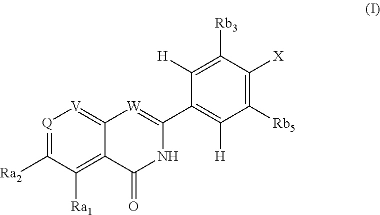
or a stereoisomer, tautomer, pharmaceutically acceptable salt, or hydrate thereof, wherein:
Q is selected from N and CRa₃;
V is selected from N and CRa₄;
W is selected from N and CH;
X is selected from OH, SH, NH₂, S(O)H, S(O)₂H, S(O)₂NH₂, S(O)NH₂, NHAc, and NHSO₂Me;
Ra₁, Ra₃, and Ra₄ are independently selected from hydrogen, C₁-C₆ alkyl, C₁-C₆ alkoxy, C₃-C₆ cycloalkyl, and halogen;
Ra₂ is selected from hydrogen, C₁-C₆ alkyl, C₁-C₆ alkoxy, C₃-C₆ cycloalkyl, amino, amide, and halogen;
Rb₃ and Rb₅ are independently selected from hydrogen, halogen, C₁-C₆ alkyl, C₃-C₆ cycloalkyl, and C₁-C₆ alkoxy;
provided that at least one of Ra₁, Ra₂, Ra₃, and Ra₄ is not hydrogen.

2. The method according to claim 1, wherein:
Q is CRa₃;
X is selected from OH, NH₂, S(O)₂NH₂, NHAc, and NHSO₂Me;
Ra₁ is selected from C₁-C₆ alkoxy;
Ra₂ is selected from hydrogen, C₁-C₆ alkoxy, amino, amide, and C₁-C₆ alkyl;
Ra₃ and Ra₄ are independently selected from hydrogen and C₁-C₆ alkoxy;
Rb₃ and Rb₅ are independently selected from C₁-C₆ alkyl and halogen.

3. The method according to claim 2, wherein:
Ra₃ is selected from hydrogen, methoxy, 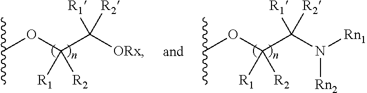
wherein
n is 0, 1, 2, or 3;
R₁, R₁′, R₂, and R₂′ are independently selected from hydrogen, C₁-C₃ alkyl, cyclopropyl, and halogen wherein if n is 1, then R₂ and R₂′, R₁ and R₁′, R₁ and R₂′, or R₂ and R₁′ may form a double bond, wherein said double bond can be cis, trans, or a mixture thereof;
Rx is selected from C₁-C₆ alkyl, C₃-C₆ cycloalkyl, and aryl; and
Rn₁ and Rn₂ are independently selected from C₁-C₆ alkyl, C₃-C₆ cycloalkyl, and aryl.

4. The method according to claim 2, wherein:
Ra₃ is selected from hydrogen, methoxy, 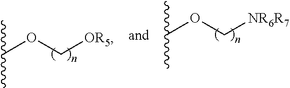
wherein
n is 1, 2, or 3;
R₅ is selected from C₁-C₆ alkyl substituted with one or more groups selected from methyl, phenyl, and pyridinyl; and
R₆ and R₇ are independently selected from unsubstituted C₁-C₆ alkyl.

5. The method according to claim 4, wherein Ra₃ is selected from hydrogen, methoxy, 2-methoxy-ethoxy, 2-dimethylamino-ethoxy, 2-benzyloxy-ethoxy, and 2-(pyridin-3-ylmethoxy)ethoxy.

6. The method according to claim 2, wherein Ra₄ is selected from hydrogen and unsubstituted C₁-C₆ alkoxy.

7. The method according to claim 6, wherein Ra₄ is selected from hydrogen and methoxy.

8. The method according to claim 2, wherein X is OH.

9. The method according to claim 2, wherein:
Ra₁ is selected from methoxy, 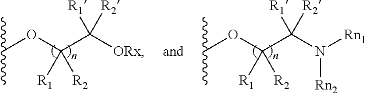
wherein
n is 0, 1, 2, or 3;
R₁, R₁′, R₂, and R₂′ are independently selected from hydrogen, C₁-C₃ alkyl, cyclopropyl, and halogen wherein if n is 1, then R₂ and R₂′, R₁ and R₁′, R₁ and R₂′, or R₂ and R₁′ may form a double bond, wherein said double bond can be cis, trans, or a mixture thereof;
Rx is selected from C₁-C₆ alkyl, C₃-C₆ cycloalkyl, and aryl; and
Rn₁ and Rn₂ are independently selected from C₁-C₆ alkyl, C₃-C₆ cycloalkyl, and aryl.

10. The method according to claim 2, wherein: 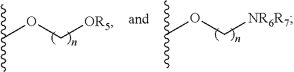
Ra₁ is selected from methoxy,
n is 1, 2, or 3; and
R₅, R₆ and R₇ are independently selected from unsubstituted C₁-C₆ alkyl.

11. The method according to claim 10, wherein Ra₁ is selected from methoxy, 2-methoxy-ethoxy, and 2-dimethylamino-ethoxy.

12. The method according to claim 2, wherein:
Ra₂ is selected from hydrogen, unsubstituted C₁-C₆ alkoxy, NHR₉, and C₁-C₆ alkyl substituted with heterocycle or amino; and
R₉ is selected from acyl, and heteroaryl.

13. The method according to claim 12, wherein Ra₂ is selected from hydrogen, methoxy, acetamido, morpholin-4-ylmethyl, pyridin-2-ylamino, (4-methylpiperazin-1-yl)methyl, and methanesulfonamido.

14. The method according to claim 1, wherein Rb₃ and Rb₅ are independently selected from unsubtituted C₁-C₆ alkyl and halogen.

15. The method according to claim 14, wherein Rb₃ and Rb₅ are independently selected from methyl, tert-butyl, fluorine, and chlorine.

16. The method according to claim 1, wherein the at least one compound of Formula I is selected from:
3-(3-fluoro-4-hydroxyphenyl)-5-methoxyisoquinolin-1(2H)-one;
3-(4-hydroxy-3,5-dimethylphenyl)-6,8-dimethoxyisoquinolin-1(2H)-one;
2-(4-hydroxy-3,5-dimethylphenyl)-5,7-dimethoxyquinazolin-4(3H)-one;
7-(4-hydroxy-3,5-dimethylphenyl)-2,4-dimethoxy-1,6-naphthyridin-5(6H)-one;
2-(3,5-di-tert-butyl-4-hydroxyphenyl)-5,7-dimethoxyquinazolin-4(3H)-one;
2-(3-chloro-4-hydroxyphenyl)-5,7-dimethoxyquinazolin-4(3H)-one;
2-(4-hydroxy-3,5-dimethylphenyl)-6,7-dimethoxyquinazolin-4(3H)-one;
N-(2-(4-hydroxy-3,5-dimethylphenyl)-4-oxo-3,4-dihydroquinazolin-6-yl)acetamide;
2-(4-hydroxy-3,5-dimethylphenyl)-6-(morpholinomethyl)quinazolin-4(3H)-one;
2-(4-hydroxy-3,5-dimethylphenyl)-5,7-dimethoxypyrido[2,3-d]pyrimidin-4(3H)-one;
2-(4-hydroxy-3,5-dimethylphenyl)-5,7-dimethoxy-6-(morpholinomethyl)quinazolin-4(3H)-one;
5-(2-dimethylamino-ethoxy)-2(4-hydroxy-3,5-dimethylphenyl)-7-methoxy-3H-quinazolin-4-one;
2-(4-hydroxy-3,5-dimethyl-phenyl)-7-methoxy-5-(2-methoxy-ethoxy)-3H-quinazolin-4-one;
7-(2-amino-ethoxy)-2-(4-hydroxy-3,5-dimethyl-phenyl)-5-methoxy-3H-quinazolin-4-one;
2-(4-hydroxy-3,5-dimethyl-phenyl)-5-methoxy-7-(2-methoxy-ethoxy)-3H-quinazolin-4-one;
7-(2-benzyloxy-ethoxy)-2-(4-hydroxy-3,5-dimethyl-phenyl)-5-methoxy-3H-quinazolin-4-one;
2-(4-hydroxy-3,5-dimethylphenyl)-5-methoxy-7-[2-(pyridin-3-ylmethoxy)ethoxy]-3H-quinazolin-4-one;
7-(2-dimethylamino-ethoxy)-2-(4-hydroxy-3,5-dimethylphenyl)-3H-quinazol in-4-one;
2-(4-hydroxy-3,5-dimethyl-phenyl)-6-(pyridin-4-ylamino)-3H-quinazol in-4-one;
2-(4-hydroxy-3,5-dimethyl-phenyl)-6-(pyridin-2-ylamino)-3H-quinazol in-4-one;
2-(4-hydroxy-3,5-dimethylphenyl)-6-(4-methylpiperazin-1-yl)methyl)quinazolin-4(3H)-one;
N-((2-(4-hydroxy-3,5-dimethylphenyl)-4-oxo-3,4-dihydroquinazolin-6-yl)methyl)methanesulfonamide, and
tatutomers, stereoisomers, pharmaceutically acceptable salts, and hydrates thereof.

17. A method of inhibiting BET (bromodomain and extra terminal domain protein) proteins comprising administering a therapeutically effective amount of at least one compound of Formula II: 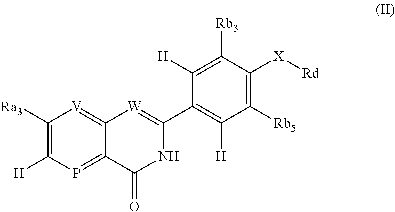
or a stereoisomer, tautomer, pharmaceutically acceptable salt, or hydrate thereof, wherein:
P is selected from N and CRa₁;
V is selected from N and CH;
W is selected from N and CH;
X is selected from O, S, CH₂, and NH;
Ra₁ and Ra₃ are independently selected from hydrogen, C₁-C₆ alkyl, C₁-C₆ alkoxy, C₃-C₆ cycloalkyl, and halogen;
Rb₃ and Rb₅ are independently selected from hydrogen, C₁-C₆ alkyl, C₃-C₆ cycloalkyl, C₁-C₆ alkoxy, halogen, and amino;
Rd is selected from C₁-C₆ alkyl, C₁-C₆ alkoxy, and C₃-C₆ cycloalkyl, wherein Rd may be connected to Rb₃ or Rb₅ to form a heterocycle,
provided that
at least one of Ra₁ and Ra₃ is not hydrogen; and
if —XRd is —OCH₂CH₂OH, then Rb₃ is not pyrrolidine.

18. The method according to claim 17, wherein:
P is CRa₁;
Ra₁ is selected from C₁-C₆ alkyl, C₁-C₆ alkoxy, and halogen;
Ra₃ is independently selected from hydrogen, C₁-C₆ alkoxy, C₁-C₆ alkyl, and halogen; and
Rb₃ and Rb₅ are independently selected from hydrogen, C₁-C₆ alkyl, C₃-C₆ cycloalkyl, C₁-C₆ alkoxy, halogen, and amino.

19. The method according to claim 18, wherein Ra₁ is selected from unsubstituted C₁-C₆ alkyl and unsubstituted C₁-C₆ alkoxy.

20. The method according to claim 19, wherein Ra₁ is selected from methyl, ethyl, methoxy, and ethoxy.

21. The method according to claim 18, wherein:
Ra₃ is selected from selected from hydrogen, methoxy, unsubstituted C₁-C₆ alkyl, halogen, and 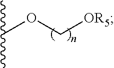
n is 1, 2, or 3; and
R₅ is C₁-C₆ alkyl substituted with phenyl or heteroaryl.

22. The method according to claim 21, wherein Ra₃ is selected from selected from hydrogen, methoxy, chlorine, fluorine, isopropoxy, methyl, 2-benzyloxy-ethoxy, and 2-(pyridin-3-ylmethoxy)ethoxy.

23. The method according to claim 18, wherein: 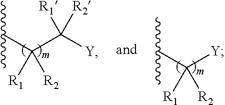
Rd is selected from C₁-C₆ alkoxy, C₃-C₆ cycloalkyl,
m is selected from 1, 2, or 3;
R₁, R₁′, R₂, and R₂′ are independently selected from hydrogen, fluorine, C₁-C₆ alkyl, hydroxyl, —NH₂, and C₁-C₆ alkoxy wherein R₂ and R₂′ may be eliminated to form a double bond;
Y is selected from OH, SH, NH₂, -Oalkyl, -Oaryl, —CH₂aryl, —C(O)NHalkyl, —C(O)N(alkyl)₂, —C(O)NHaryl, —NHacyl, —NHalkyl, —NHS(O)₂alkyl, —N(alkyl)₂, —NHS(O)₂N(alkyl)₂, —NHCN, and —NHC(O)N(alkyl)₂, —NHheterocyclyl, and heterocyclyl; and
Rd may be connected to Rb₃ or Rb₅ to form a heterocycle,
provided that for —N(alkyl)₂ the alkyl chains cannot be joined to form an aryl or heterocyclic ring.

24. The method according to claim 23, wherein Rd is connected to Rb₃ or Rb₅ to form a heterocycle selected from substituted furanyl or substituted pyrrolyl.

25. The method according to claim 24, wherein said heterocycle is selected from 2-hydroxymethyl-furan-5-yl or 2-(4,5-dihydro-1H-pyrrol-2-yl)ethanol.

26. The method according to claim 18, wherein X-Rd is selected from 2-hydroxy-2-methylpropoxy, 2-hydroxyethoxy, methoxy, benzyloxyethoxy, 2,3-dihydroxypropoxy, aminocarbonylethoxy, methylaminocarbonylethoxy, (4-methoxyphenyl)aminocarbonylethoxy, benzylaminocarbonylethoxy, 4-hydroxybutoxy, (5-phenyl-4H-[1,2,4]triazol-3-ylamino)ethoxy, (3-methyl-[1,2,4]oxadiazol-5-ylamino)ethoxy, methylcarbonylaminoethoxy, methylcarbonylaminomethyl, (2,2,2-trifluoro-ethylamino)ethoxy, methanesulfonylaminoethoxy, isobutyrylaminoethoxy, methylaminoethoxy, isopropylsulfonylaminoethoxy, methylcarbonylaminoethoxy, dimethylaminoethoxy, N-(2-hydroxyethyl)-N-methylacetamide, formamide-N-2-ethoxy, methylformamide-N-2-ethoxy, dimethylsulfonylaminoethoxy, cyanoaminoethoxy, (5-methylisoxazol-3-ylamino)ethoxy, (pyrimidin-2-ylamino)ethoxy, (isoxazol-3-ylamino)ethoxy, (4,6-dimethoxypyrimidin-2-ylamino)ethoxy, 3-hydroxypropyl, and 2-hydroxyethyl.

27. The method according to claim 26, wherein X-Rd is selected from hydroxyethoxy, methylcarbonylaminoethoxy, (4-methoxyphenyl)aminocarbonylethoxy, and isobutyrylaminoethoxy.

28. The method according to claim 17, wherein the at least one compound of Formula II is selected from:
3-(4-(2-hydroxy-2-methylpropoxy)-3,5-dimethylphenyl)-6,8-dimethoxyisoquinolin-1(2H)-one;
2-(4-(2-hydroxyethoxy)-3,5-dimethylphenyl)-5,7-dimethoxyquinazolin-4(3H)-one;
5,7-dimethoxy-2-(4-methoxyphenyl)quinazolin-4(3H)-one;
2-(4-(2-hydroxyethoxy)-3,5-dimethylphenyl)-6,7-dimethoxyquinazolin-4(3H)-one;
5,7-dimethoxy-2-(4-methoxy-3-(morpholinomethyl)phenyl)quinazolin-4(3H)-one;
2-(4-(2-hydroxyethoxy)-3,5-dimethylphenyl)-5,7-dimethoxypyrido[2,3-d]pyrimidin-4(3H)-one;
N-(2-(4-(2-hydroxyethoxy)-3,5-dimethylphenyl)-4-oxo-3,4-dihydroquinazolin-6-yl)acetamide;
2-(4-(2-(benzyloxy)ethoxy)-3,5-dimethylphenyl)-5,7-dimethoxypyrido[2,3-d]pyrimidin-4(3H)-one;
2-(4-(2-hydroxyethoxy)-3,5-dimethylphenyl)-5,7-dimethylpyrido[2,3-d]pyrimidin-4(3H)-one;
5,7-difluoro-2-(4-(2-hydroxyethoxy)-3,5-dimethylphenyl)quinazolin-4(3H)-one;
5,7-dichloro-2-(4-(2-hydroxyethoxy)-3,5-dimethylphenyl)quinazolin-4(3H)-one;
2-[4-(2-hydroxy-ethoxy)-3,5-dimethyl-phenyl]-5,7-diisopropoxy-3H-quinazol in-4-one;
2-[4-(2,3-Dihydroxy-propoxy)-3,5-dimethyl-phenyl]-5,7-dimethoxy-3H-quinazolin-4-one;
2-[4-(2-hydroxy-ethoxy)-phenyl]-5,7-dimethoxy-3H-quinazolin-4-one;
2-[4-(2-hydroxy-ethoxy)-naphthalen-1-yl]-5,7-dimethoxy-3H-quinazolin-4-one;
2-(2-hydroxymethyl-benzofuran-5-yl)-5,7-dimethoxy-3H-quinazolin-4-one;
7-(2-benzyloxy-ethoxy)-2-[4-(2-hydroxy-ethoxy)-3,5-dimethyl-phenyl]-5-methoxy-3H-quinazolin-4-one;
7-(2-benzyloxy-ethoxy)-2-(2-hydroxymethyl-benzofuran-5-yl)-5-methoxy-3H-quinazolin-4-one;
2-[4-(5,7-dimethoxy-4-oxo-3,4-dihydro-quinazolin-2-yl)-2,6-dimethyl-phenoxy]-acetamide;
2-[4-(5,7-dimethoxy-4-oxo-3,4-dihydro-quinazolin-2-yl)-2,6-dimethyl-phenoxy]-N-methyl-acetamide;
2-[4-(5,7-Dimethoxy-4-oxo-3,4-dihydro-quinazolin-2-yl)-2,6-dimethyl-phenoxy]-N-(4-methoxy-phenyl)-acetamide;
N-benzyl-2-[4-(5,7-dimethoxy-4-oxo-3,4-dihydroquinazolin-2-yl)-2,6-dimethylphenoxy]acetamide;
2-[4-(4-hydroxy-butoxy)-3,5-dimethyl-phenyl]-5,7-dimethoxy-3H-quinazolin-4-one;
2-(4-(2-hydroxyethoxy)-3,5-dimethylphenyl)-5-methoxyquinazolin-4(3H)-one;
7-chloro-2-(4-(2-hydroxyethoxy)-3,5-dimethylphenyl)quinazolin-4(3H)-one;
5-chloro-2-(4-(2-hydroxyethoxy)-3,5-dimethylphenyl)quinazolin-4(3H)-one;
2-(4-(2-hydroxyethoxy)-3,5-dimethylphenyl)-7-methoxyquinazolin-4(3H)-one;
5,7-dimethoxy-2-(4-methoxy-3,5-dimethylphenyl)quinazolin-4(3H)-one;
2-(4-(2-hydroxyethoxy)-3-methylphenyl)-5,7-dimethoxyquinazolin-4(3H)-one;
5,7-Dimethoxy-2-{3-methyl-4-[2-(5-phenyl-4H-[1,2,4]triazol-3-ylamino)-ethoxy]-phenyl}-3H-quinazolin-4-one;
2-{3,5-Dimethyl-4-[2-(3-methyl-[1,2,4]oxadiazol-5-ylamino)-ethoxy]-phenyl}-5,7-dimethoxy-3H-quinazolin-4-one;
N-{2-[4-(5,7-dimethoxy-4-oxo-3,4-dihydro-pyrido[2,3-d]pyrimidin-2-yl)-2,6-dimethyl-phenoxy]-ethyl}-acetamide;
N-(4-(5,7-dimethoxy-4-oxo-3,4-dihydroquinazolin-2-yl)-2,6-dimethylbenzyl)acetamide;
N-[4-(5,7-dimethoxy-4-oxo-3,4-dihydro-pyrido[2,3-d]pyrimidin-2-yl)-2,6-dimethyl-benzyl]-acetamide;
2-{3,5-Dimethyl-4-[2-(2,2,2-trifluoro-ethylamino)-ethoxy]-phenyl}-5,7-dimethoxy-3H-quinazolin-4-one;
N-{2-[4-(6,8-Dimethoxy-1-oxo-1,2-dihydro-isoquinolin-3-yl)-2,6-dimethyl-phenoxy]-ethyl}-formamide;
N-(2-(4-(5,7-dimethoxy-4-oxo-3,4-dihydroquinazolin-2-yl)-2,6-dimethylphenoxy)ethyl)methanesulfonamide;
N-(2-(4-(5,7-dimethoxy-4-oxo-3,4-dihydroquinazolin-2-yl)-2,6-dimethylphenoxy)ethyl)-4-methoxybenzamide;
N-(2-(4-(5,7-dimethoxy-4-oxo-3,4-dihydroquinazolin-2-yl)-2,6-dimethylphenoxy)ethyl)acetamide;
N-(2-(4-(5,7-dimethoxy-4-oxo-3,4-dihydroquinazolin-2-yl)-2,6-dimethylphenoxy)ethyl)isobutyramide;
2-(3,5-dimethyl-4-(2-(methylamino)ethoxy)phenyl)-5,7-dimethoxyquinazolin-4(3H)-one;
N-(2-(4-(5,7-dimethoxy-4-oxo-3,4-dihydroquinazolin-2-yl)-2,6-dimethylphenoxy)ethyl)propane-2-sulfonamide;
2-(4-(2-(isopropylamino)ethoxy)-3,5-dimethylphenyl)-5,7-dimethoxyquinazolin-4(3H)-one;
N-(2-(4-(5,7-dimethoxy-4-oxo-3,4-dihydroquinazolin-2-yl)-2-methylphenoxy)ethyl)acetamide;
N-(2-(4-(5,7-dimethoxy-4-oxo-3,4-dihydroquinazolin-2-yl)-2-methylphenoxy)ethyl)isobutyramide;
N-(2-(4-(5,7-dimethoxy-4-oxo-3,4-dihydroquinazolin-2-yl)-2-methylphenoxy)ethyl)methanesulfonamide;
2-(4-(2-(dimethylamino)ethoxy)-3,5-dimethylphenyl)-5,7-dimethoxyquinazolin-4(3H)-one;
N-(2-(4-(5,7-dimethoxy-4-oxo-3,4-dihydroquinazolin-2-yl)-2,6-dimethylphenoxy)ethyl)-N-methylacetamide;
N-(2-(4-(5,7-dimethoxy-4-oxo-3,4-dihydroquinazolin-2-yl)-2,6-dimethylphenoxy)ethyl)formamide;
N-(2-(4-(5,7-dimethoxy-4-oxo-3,4-dihydroquinazolin-2-yl)-2,6-dimethylphenoxy)ethyl)-N-methylformamide;
N-(2-(4-(5,7-Dimethoxy-4-oxo-3,4-dihydroquinazolin-2-yl)-2,6-dimethylphenoxy)ethyl)dimethylamino-N-sulfonamide;
N-(2-(4-(5,7-dimethoxy-4-oxo-3,4-dihydroquinazolin-2-yl)-2,6-dimethylphenoxy)ethyl)cyanamide;
2-(3,5-dimethyl-4-(2-(5-methylisoxazol-3-ylamino)ethoxy)phenyl)-5,7-dimethoxyquinazolin-4(3H)-one;
2-(3,5-dimethyl-4-(2-(pyrimidin-2-ylamino)ethoxy)phenyl)-5,7-dimethoxyquinazolin-4(3H)-one;
2-(4-(2-(isoxazol-3-ylamino)ethoxy)-3,5-dimethylphenyl)-5,7-dimethoxyquinazolin-4(3H)-one;
2-(4-(2-(4,6-dimethoxypyrimidin-2-ylamino)ethoxy)-3,5-dimethylphenyl)-5,7-dimethoxyquinazolin-4(3H)-one;
2-[4-(3-hydroxy-propyl)-3,5-dimethoxyphenyl]-5,7-dimethoxy-3H-quinazolin-4-one;
2-[4-(3-hydroxy-propyl)-3-methoxy-phenyl]-5,7-dimethoxy-3H-quinazolin-4-one; and
2-[2-(2-hydroxyethyl)-1H-indol-6-yl]-5,7-dimethoxy-3H-quinazolin-4-one, and
tautomers, stereoisomers, pharmaceutically acceptable salts, and hydrates thereof.

29. The method according to claim 1, wherein the therapeutically effective amount of the compound is administered with at least one pharmaceutically acceptable carrier in a pharmaceutically acceptable composition.

30. The method according to claim 17, wherein the therapeutically effective amount of the compound is administered with at least one pharmaceutically acceptable carrier in a pharmaceutically acceptable composition.

31. The method according to claim 1, wherein the compound of Formula 1 is administered to treat or prevent a cancer selected from cancers that exhibit c-myc overexpression, cancers that overexpress n-myc, cancers that that rely on the recruitment of p-TEFb to regulate activated oncogenes, Burkitt's lymphoma, acute myelogenous leukemia, multiple myeloma, aggressive human medulloblastoma, hematological, epithelial cancers, lung cancers, breast cancers, colon carcinomas, midline carcinomas, mesenchymal tumors, hepatic tumors, renal tumors, and neurological tumors.

32. The method of claim 31, wherein the compound of Formula I is administered in combination with another anti-cancer agent selected from the group consisting of bortezomib, thalidomide, dexamethasone, 5-azacitidine, decitabine, vorinostat, cyclophosphamide, a PI3K or mTOR inhibitor, rapamycin or a rapamycin analog, a gamma secretase inhibitor, an AMPK inducer, metformin, phenformin, an ornithine decarboxylase inhibitor, and difluoromethylornithine.
